(19) United States
(12) Patent Application Publication
(10) Pub. No.: US 2023/0218654 A1
(43) Pub. Date: Jul. 13, 2023

(54) COMPOSITIONS AND METHODS FOR TREATING HYPERGLYCEMIA IN TYPE-2 DIABETES

(71) Applicant: The Board of Regents of the university of Oklahoma, Norman, OK (US)

(72) Inventor: Tiangang Li, Edmond, OK (US)

(21) Appl. No.: 18/181,016

(22) Filed: Mar. 9, 2023

Related U.S. Application Data
(63) Continuation-in-part of application No. PCT/US2021/049851, filed on Sep. 10, 2021.
(60) Provisional application No. 63/076,662, filed on Sep. 10, 2020.

(51) Int. Cl. A61K 31/7064; A61K 31/519; A61P 3/10

(52) U.S. Cl. CPC A61K 31/7064; A61K 31/519; A61P 3/10

(57) ABSTRACT

Compositions and methods of treating hyperglycemia in a subject having such a condition are disclosed. An inhibitor compound which causes activity of Cullin RING E3 ligases (CRL) to be reduced is administered to the subject, wherein blood glucose concentration is decreased. In at least certain embodiments insulin secretion and insulin sensitization in the subject are increased. Inhibition of CRL activity, e.g., by decreasing cullin neddylation by inhibiting NEDD8-activating enzyme (NAE), or decreasing cullin activity, e.g., by inhibiting expression of cullins, is shown herein to delay IRS protein turnover in liver cells and muscle cells, thereby increasing cellular response to insulin and decreasing blood glucose. Thus, inhibition of CRLs, for example by inhibiting neddylation, is an effective method to treat hyperglycemia and insulin resistance, and to increase insulin secretion in patients with hyperglycemia, for example due to type-2 diabetes.

### CROSS-REFERENCE TO RELATED APPLICATIONS

The present application is a continuation-in-part application of International Application No. PCT/US2021/049851, filed Sep. 10, 2021; which claims priority under 35 U.S.C. § 119(e) to U.S. Serial No. 63/076,662, filed Sep. 10, 2020. The entirety of each of the above-referenced patent applications is hereby expressly incorporated herein by reference.

### STATEMENT REGARDING FEDERALLY SPONSORED RESEARCH OR DEVELOPMENT

This invention was made with government support under grant number 1R01DK102487-01 awarded by the National Institutes of Health. The government has certain rights in the invention.

### BACKGROUND

Insulin resistance is a characteristic feature of type-2 diabetes. Insulin resistance in both the liver and extrahepatic tissues, especially skeletal muscle and adipose tissue, contributes to hyperglycemia, hepatic steatosis, dyslipidemia, and metabolic disturbance. Under normal physiology, hepatic insulin signaling is activated after a meal to inhibit glucose production. Under fasting, insulin signaling is diminished, which favors hepatic glucose production. However, in hepatic insulin resistance, insulin loses its suppressive effect on hepatic glucose synthesis leading to abnormally increased hepatic glucose output that contributes significantly to elevated blood glucose in type-2 diabetes. Skeletal muscle is quantitatively the most important organ to take up glucose from the circulation during postprandial period. Muscle glucose uptake depends on insulin to promote glucose uptake transporter type 4 (GLUT4) to translocate from the intracellular compartment to the cell surface. Insulin resistance in skeletal muscle impairs insulin-dependent glucose uptake, which is the major cause of postprandial hyperglycemia in type-2 diabetic patients due to delayed glucose clearance from the circulation. Glucose uptake into white adipose tissue is also stimulated by insulin via increased GLUT4 plasma membrane translocation. Although white adipose tissue glucose uptake only accounts for a minor portion of total blood glucose clearance, insulin resistance in white adipose tissue promotes fatty acid release into the circulation. Fatty acids are preferentially taken up by the liver. This is the major cause of fatty liver disease that is commonly found in patients with obesity and type-2 diabetes.

A Cullin-RING E3 ligase (CRL) is a multi-protein complex generally consisting of a cullin protein, a RING (really interesting new gene) E3 ligase and a substrate receptor that recognizes specific substrates for ubiquitination and proteasomal degradation. Each cullin member protein serves as the scaffold of a functionally distinct CRL. In addition, mammalian cells express a large number of substrate receptors in a tissue-specific manner, which further determines the substrate specificity of a unique CRL complex. CRLs are activated upon cullin neddylation, a process of covalent conjugation of a ubiquitin-like protein called Nedd8 to a conserved lysine on a cullin protein. Neddylation results in cullin conformational change that is needed for optimal CRL assembly and function. Neddylation is mediated by a set of specialized Nedd8 E1, E2 and E3 enzymes that sequentially transfer Nedd8 to a cullin protein. Unlike protein ubiquitination, current studies support that cullin proteins are the predominant neddylation targets in mammalian cells. Recently, CRLs have emerged as novel targets for drug development due to the higher substrate selectivity and lack of broad cellular impact upon inhibition. In the last 10 years, CRLs have attracted major attention in cancer research owing to CRL regulation of oncogenes and tumor suppressors. The Nedd8-activating E1 enzyme (NAE1) is the only known Nedd8 E1 enzyme. MLN4924 (Pevonedistat) is the first in-class small molecule inhibitor of NAE1 and has entered Phase-I/II clinical trials for various cancer treatments. In contrast, the translational potential of targeting cullin neddylation for treating other diseases is still largely unknown.

Fatty acids that enter the muscle are considered a major cause of muscle insulin resistance via activation of cellular stress kinases that inactivate insulin signaling. Therapeutics and treatments for reducing insulin resistance and thereby reducing hyperglycemia and other outcomes of insulin resistance are highly desired. It is to such treatments that the present disclosure is directed

### BRIEF DESCRIPTION OF THE DRAWINGS

Several embodiments of the present disclosure are hereby illustrated in the appended drawings. It is to be noted however, that the appended drawings only illustrate several typical embodiments and are therefore not intended to be considered limiting of the scope of the inventive concepts disclosed herein.

FIG. 1 shows a schematic representation of a mechanism of insulin resistance at the insulin receptor substrate (IRS) level. Under normal conditions, insulin binding to insulin receptor results in the recruitment of IRS and IRS tyrosine (Y) phosphorylation, which is a key event in insulin-stimulated signal transduction in cells. In type-2 diabetes, prolonged insulin exposure at high concentrations and abnormal activation of nutrient kinases (i.e. mTOR/S6K)/stress kinases (JNK/PKC) cause serine/threonine (S/T) phosphorylation of IRS, which is then recognized by Cullin RING E3 ligases (CRLs) for ubiquitination and degradation. Impaired IRS function contributes significantly to hepatic and muscle insulin resistance, which is the major cause of hyperglycemia in type-2 diabetes. MLN4924 inhibits CRL resulting in reduced degradation of IRS and improved insulin sensitivity.

FIG. 2 shows a cartoon of a structure of a CRL complex. Ub = ubiquitin. N8 = Nedd8. A CRL complex consists of a cullin scaffold, a RING E3 ligase and a substrate adaptor that recognizes specific substrates (e.g., IRS) for ubiquitination and subsequent proteasomal degradation. CRLs are activated upon cullin neddylation, a process of covalent conjugation of a ubiquitin-like protein called Nedd8 to a conserved lysine on a cullin protein. This unique feature has allowed for the development of neddylation inhibitors (e.g., MLN4924; TAS4464) of the only mammalian Nedd8-activating E1 enzyme (NAE1) that mediates cullin neddylation.

FIG. 3 demonstrates that human and murine Nonalcoholic steatohepatitis/Nonalcoholic fatty liver disease (NASH/NAFLD) shows cullin hyper-neddylation and impaired IRS expression. A. Human NASH/NAFLD shows cullin hyper-neddylation. Histology confirmed normal and NASH livers. B. Murine NAFLD shows cullin hyper-neddylation in male C57BL/6J mice fed chow (C) or Western diet (WD) for 8 weeks. C. Murine NAFLD shows cullin hyper-neddylation and impaired IRS expression in male C57BL/6J mice fed C or WD for 12 weeks.

FIG. 4A shows that MLN4924 inhibits liver cullin neddylation and enhances liver insulin signaling. A. Western blot of liver lysates. Chow-fed male C56BL/6J mice were injected with 60 mg/kg MLN4924 or Vehicle (10% 2HO-β-cyclodextrin) at 5 pm on day 1 and 9 am on day 2, and sacrificed at 3 pm after 6 h fast. Upper arrow: Neddylated Cul3; Lower arrow: de-neddylated Cul3. Veh=vehicle; IP=intraperitoneal; SQ=subcutaneous.

FIG. 4B shows that MLN4924 acutely decreases hepatic glucose production. Male C56BL/6J mice fed chow diet were treated with MLN4924 as in FIG. 4A via SQ injection. Pyruvate tolerance test (PTT) was performed at 3 pm after 6 h fast. “*”, p<0.05, vs. Vehicle. N=4-5.

FIG. 4C shows that MLN4924 acutely decreases hepatic glucose production. Male C56BL/6J mice were fed WD for 4 weeks to induce insulin resistance. Mice were then treated with MLN4924 or Vehicle as in FIG. 4A via SQ injection. Pyruvate tolerance test (PTT) was performed at 3 pm after 6 h fast. “*”, p<0.05, vs. Vehicle. N=4-5.

FIG. 5A shows that MLN4924 treatment did not affect body weight in mice fed C or WD over 16 weeks. Mice fed WD became obese and insulin resistant. For both cohorts, body weight was measured weekly. Male C57BL/6J mice were fed C or WD for 16 weeks. Vehicle or MLN4924 (60 mg/kg) was administered via SQ injection twice/week for 16 weeks. Results are mean ±SEM. n=5-7. “*”, vs C+Veh; “#”, vs. WD+Veh, p<0.05.

FIG. 5B shows that chronic MLN4924 treatment significantly reduced blood glucose in male C57BL/6J mice fed WD for 16 weeks independent of obesity. Blood glucose was measured after a 6 h fast (from 9 am-3 pm). Treatments were as in FIG. 5A. Results are mean ±SEM. n=5-7. “*”, vs C+Veh; “#”, vs. WD+Veh, p<0.05.

FIG. 5C shows that MLN4924 treatment significantly reduced liver triglycerides (TG) in male C57BL/6J mice fed WD over 16 weeks. Treatments were as in FIG. 5A. Results are mean ±SEM. n=5-7. “*”, vs C+Veh; “#”, vs. WD+Veh, p<0.05.

FIG. 5D shows that MLN4924 treatment significantly reduced liver cholesterol in male C57BL/6J mice fed WD over 16 weeks. Treatments were as in FIG. 5A. Results are mean ±SEM. n=5-7. “*”, vs C+Veh; “#”, vs. WD+Veh, p<0.05.

FIG. 5E shows that MLN4924 treatment significantly reduced plasma levels of the liver injury marker alanine aminotransferase (ALT - a.k.a.. alanine transaminase) elevated by WD feeding in male C57BL/6J mice over 16 weeks. Treatments were as in FIG. 5A. Results are mean ±SEM. n=5-7. “*”, vs C+Veh; “#”, vs. WD+Veh, p<0.05.

FIG. 5F shows that MLN4924 treatment did not affect diet-induced obesity in mice fed C or WD over 16 weeks when MLN4924 treatment was initiated after 8 weeks of feeding. MLN4924 (60 mg/kg, SQ) was given every other day for the last 8 weeks of the 16-week feeding. For both cohorts, body weight was measured weekly. Results are mean ±SEM. n=5-7. “*”, vs C+Veh; “#”, vs. WD+Veh, p<0.05. ns= Not significant.

FIG. 5G shows that MLN4924 treatment had no significant effect on adiposity as measured by gonadal fat weight in male C57BL/6J mice fed C or WD over 16 weeks. Treatments were as in FIG. 5F. Results are mean ±SEM. n=5-7. “*”, vs C+Veh; “#”, vs. WD+Veh, p<0.05.

FIG. 5H shows that MLN4924 treatment MLN4924 treatment lowers 6 h fasting blood glucose in male C57BL/6J mice fed WD for 16 weeks. Blood glucose was measured after a 6 h fast (from 9 am-3 pm). Treatments were as in FIG. 5F. Results are mean ±SEM. n=5-7. “*”, vs C+Veh; “#”, vs. WD+Veh, p<0.05.

FIG. 5I shows that MLN4924 treatment had no significant effect on serum free fatty acids (FFA) in male C57BL/6J mice fed C or WD over 16 weeks. Treatments were as in FIG. 5F. Results are mean ±SEM. n=5-7. “*”, vs C+Veh; “#”, vs. WD+Veh, p<0.05.

FIG. 5J shows that MLN4924 treatment had no significant effect on liver glycogen in male C57BL/6J mice fed C or WD over 16 weeks. Treatments were as in FIG. 5F. Results are mean ±SEM. n=5-7. “*”, vs C+Veh; “#”, vs. WD+Veh, p<0.05.

FIG. 5K shows the effect of MLN4924 treatment on circulating insulin in one experiment on male C57BL/6J mice fed C or WD over 16 weeks. Treatments were as in FIG. 5F. Results are mean ±SEM. n=5-7. “*”, vs C+Veh; “#”, vs. WD+Veh, p<0.05.

FIG. 5L shows that MLN4924 treatment significantly reduced liver triglycerides (TG) (hepatic steatosis) in male C57BL/6J mice fed WD for 16 weeks. Treatments were as in FIG. 5F. Results are mean ±SEM. n=5-7. “*”, vs C+Veh; “#”, vs. WD+Veh, p<0.05.

FIG. 5M shows that MLN4924 treatment significantly reduced serum aspartate aminotransferase (AST - a.k.a. aspartate transaminase) levels elevated by WD feeding in male C57BL/6J mice fed WD over 16 weeks. Treatments were as in FIG. 5F. Results are mean ±SEM. n=5-7. “*”, vs C+Veh; “#”, vs. WD+Veh, p<0.05.

FIG. 5N shows Western blots which demonstrate that MLN4924 treatment increases liver IRS protein abundance and Protein kinase B (AKT) phosphorylation (P-AKT). Male C57BL/6J mice were fed chow or WD for 16 weeks. Vehicle or MLN4924 treatment (60 mg/kg, SQ, once every two days) was initiated after 8 weeks of WD feeding. Analyses were performed after 16 weeks of feeding. Results are mean ± SEM. n=5-8. “*”, vs C+Veh; “#”, vs. WD+Veh, p<0.05. Normalized densitometry was shown in low panel bar graphs.

FIG. 6A shows Western blots that demonstrate that MLN4924 enhances hepatocyte insulin responsiveness by delaying feedback IRS degradation in AML12 mouse hepatocytes. MLN4924 increases IRS protein and AKT activation in the presence of insulin. Mouse AML 12 hepatocytes were serum starved for 16 h. Cells were then pre-treated with MLN4924 for 1 h followed by 100 nM insulin stimulation for 4 h. Western blot was then performed to evaluate insulin signaling activation.

FIG. 6B shows Western blots that demonstrate the effects of MLN4924 on insulin signaling sensitization in primary human hepatocytes. Cells were pre-treated with increasing dose of MLN4924 for 1 h followed by 100 nM insulin stimulation for 4 h. Western blot was then performed to evaluate insulin signaling activation reflected by AKT phosphorylation.

FIG. 6C shows comparative results of control vs. MLN4924 treatment on amount of IRS 1 protein in primary human hepatocytes. Cells were treated with MLN4924 for 8 h. IRS1 band densitometry is shown as mean ± SEM of 8-9 independent batches. “* ”, vs. Vehicle, p less than 0.05.

FIG. 6D shows comparative results of control vs. MLN4924 treatment on amount of IRS2 protein in primary human hepatocytes. Cells were treated with MLN4924 for 8 h. IRS2 band densitometry is shown as mean ± SEM of 8-9 independent batches. “* ”, vs. Vehicle, p less than 0.05.

FIG. 6E shows Western blot results of high insulin-induced IRS1 and IRS2 protein degradation is prevented by MLN4924 treatment in AML 12 cells. AML 12 cells were serum starved for 16 h. Cells were then pre-treated with 500 nM MLN4924 or vehicle (DMSO) for 1 h, followed by 100 µg/ml cycloheximide (CHX) and 100 nM insulin treatment.

FIG. 6F shows Western blots demonstrating MLN4924 effects on enhancing insulin signaling activation in AML12 cells. AML12 cells were serum starved for 16 h. Cells were then pre-treated with 500 nM MLN4924 for 1 h followed by 100 nM insulin stimulation in time course.

FIG. 6G shows Western blots demonstrating MLN4924 effects on enhancing insulin signaling activation in primary mouse hepatocytes after pretreatment with MLN4924 (500 nM) for 1 h followed by 100 nM insulin treatment in time course.

FIG. 7A a shows the knockdown of Cull, Cul2, Cul4B and Cul5 ligases by specific anti-Cul siRNAs in AML12 cells. Mean ± SD of 3 independent experiments. “*”, p<0.05 (unpaired t-test), vs. siCon (control siRNA). Specific siRNAs strongly knocked down the targeted cullin without affecting the mRNA expression of other cullin members.

FIG. 7Ab shows the knockdown of Cul3, Cul4 A, and Cul7 ligases by specific anti-Cul siRNAs in AML12 cells. Mean ± SD of 3 independent experiments. “*”, p<0.05 (unpaired t-test), vs. siCon (control siRNA). Specific siRNAs strongly knocked down the targeted cullin without affecting the mRNA expression of other cullin members.

FIG. 7B shows that knockdown of Cull by Cull-specific siRNA in AML12 cells that were serum starved for 16 h followed by 100 nM insulin stimulation in time course increased IRS protein abundance and enhanced AKT phosphorylation. Knockdown of Cull ligases recapitulates the insulin sensitizing effect of MLN4924. Knockdown of Cull increases IRS1 protein abundance under basal culture condition or upon treatment with insulin. Blots are representative of 2-4 independent experiments.

FIG. 7C shows that knockdown of Cul3 by Cul3-specific siRNA in AML12 cells that were serum starved for 16 h followed by 100 nM insulin stimulation in time course increased IRS protein abundance and enhanced AKT phosphorylation. Knockdown of Cul3 ligases recapitulates the insulin sensitizing effect of MLN4924. Knockdown of Cul3 increases IRS1 protein abundance under basal culture condition or upon treatment with insulin. Blots are representative of 2-4 independent experiments.

FIG. 7D shows that knockdown of FBXW8 by FBXW8 siRNA decreased levels of FBXW8 mRNA in AML12 cells. FBXW8 is an IRS-recognizing substrate receptor in the cullin RING E3 ligase.

FIG. 7E shows that knockdown of FBXW8 protein by FBXW8 siRNA increased abundance of IRS proteins and enhanced insulin stimulation of AKT phosphorylation in AML 12 cells. Insulin: 100 nM.

FIG. 7F shows Co-IP results of FBXW8-IRS1 protein-protein interaction in AML12 cells. V5-tagged IRS1 and Myc-tagged FBXW8 were expressed in AML12 cells. Myc-FBXW8 was immunoprecipitated by Myc antibodies and co-precipitated V5-IRS1 was detected with anti-V5 antibodies.

FIG. 8A shows that CRL inhibition by MLN4924 increases IRS proteins and enhances insulin signaling in muscle C2C12 cells. Differentiated C2C12 myocytes were pre-treated with MLN4924 (500 nM) for 1 h followed by 100 nM insulin treatment in time course. Increased IRS protein abundance and enhanced AKT phosphorylation were detected by Western blot.

FIG. 8B shows that CRL inhibition by MLN4924 enhances glucose uptake in muscle C2C12 cells. Differentiated C2C12 myocytes were treated with MLN4924 (500 nM) and then stimulated with 100 nM insulin as indicated. Glucose uptake into the cells was measured. Glucose uptake into the cells was increased in the presence of MLN4924.Results are mean+/-SD of 5 replicates. “* ”, p less than 0.05, vs Vehicle-treated control.“#”, vs. insulin treated, p less than 0.05.

FIG. 8C shows MLN4924 treatment acutely increased glucose tolerance in male C57BL/6J mice fed with C. MLN4924(60 mg/kg, SQ) was injected at 5 pm on day 1 and 9 am on day 2. Mice were fasted for 6 h and glucose tolerance test (GTT) was performed at 3 pm on day 2. These results suggest increased glucose clearance by muscle in MLN4924 treated mice. Results are mean+/- SEM (n=4-5). “*”, p less than 0.05, vs. Vehicle.

FIG. 8D shows MLN4924 treatment acutely increased glucose tolerance in male C57BL/6J mice fed 3 weeks with WD and then treated with MLN4924 (60 mg/kg, SQ) as in FIG. 8C. Results are mean+/- SEM (n=4-5). “*”, p less than 0.05, vs. Vehicle.

FIG. 9A shows Western blots that demonstrate that treatment of AML12 cells for 5 h with increasing doses of the selective NAE1 inhibitor TAS4464 effectively inhibits total neddylated cullins and neddylated Cul-1 and increases IRS protein in AML12 cells.

FIG. 9B shows that treatment of insulin resistant and obese male C57BL/6J mice with TAS4464 did not affect body weight. Mice were fed WD for 8 weeks to render them insulin resistant and obese. Mice were then treated with 45 mg/kg TAS4464 or vehicle once at 5 pm on day 1 and once at 9 am on day 2. Body weight and blood glucose were measured at 3 pm on day 2.

FIG. 9C shows that treatment of insulin resistant and obese male C57BL/6J mice with TAS4464 acutely reduced blood glucose. Mice were fed WD for 8 weeks, after which the mice were injected with vehicle or 45 mg/kg TAS4464 subcutaneously at 5 pm on day 1 and 9 am on day 2. Mice were fasted from 9 am to 3 pm on day 2 and blood glucose was measured. Results are expressed as mean ± SEM. n=S. “*”, p < 0.05, vs. Vehicle.

FIG. 10 shows chemical structures of various NAE inhibitors which can be used in accordance with the present invention. Taken from Yu, Q., Jiang, Y., and Sun Y., (2020) “Anticancer drug discovery by targeting cullin neddylation.” Acta Pharmaceutica Sinica B 10(5), 746-765.

FIG. 11 shows chemical structures of various additional NAE inhibitors which can be used in accordance with the present invention. Taken from Yu et al, 2020, op cit.

FIG. 12A is a Western blot showing that MLN4924 inhibits liver cullin neddylation and increases IRS protein abundance and insulin signaling activation. Chow-fed male C56BL/6J mice were subcutaneously (SQ) injected 60 mg/kg MLN4924 or Veh (10 % 2HO-β-cyclodextrin) at 5 pm on day 1 and 9 am on day 2. Mice were fasted from 9 am to 3 pm on day 2 and sacrificed. Upper and lower arrows indicate neddylated cullin1 and cullin3 bands, respectively.

FIG. 12A a is a graph showing normalized densitometry of the hepatic neddylated cullin band of FIG. 12A. MLN4924 reduced cullin neddylation.

FIG. 12Ab shows normalized densitometry of the hepatic neddylated cullin1 band (left panel) and the neddylated cullin3 band (right panel) of FIG. 12A. MLN4924 reduced cullin1 and 3 neddylation.

FIG. 12Ac shows normalized densitometry of the hepatic IRS1 band (left panel) and the IRS2 band (right panel) of FIG. 12A. MLN4924 increased IRS1 and IRS2 production.

FIG. 12Ad is a graph showing normalized densitometry of the hepatic phosphorylated AKT (P-AKT) band of FIG. 12A. MLN4924 increased hepatic phosphorylated AKT.

and p-AKT band densitometry is shown in the right panels.

FIG. 12B shows that acute treatment with MLN4924 decreased plasma glucose production in male C57BL/6J mice fed a chow (C) diet for 4 weeks then administered 60 mg/kg MLN4924 as described in FIG. 12A. A pyruvate tolerance test (PTT) was performed. Results are expressed as mean ± SEM (n=4-5). “*”, p<0.05, vs. Vehicle group at the same time point. Unpaired Student’s t-test was used to calculate the p value.

FIG. 12C shows that acute treatment with MLN4924 decreased plasma glucose production in male C57BL/6J mice fed a chow (C) diet for 4 weeks then administered 60 mg/kg MLN4924 as described in FIG. 12A. A glucose tolerance test (GTT) was performed. Results are expressed as mean ± SEM (n=4-5). “*”, p<0.05, vs. Vehicle group at the same time point. Unpaired Student’s t-test was used to calculate the p value.

FIG. 12D shows that acute treatment with MLN4924 decreased plasma glucose production in male C57BL/6J mice fed a Western diet (WD) for 4 weeks then administered 60 mg/kg MLN4924 as described in FIG. 12A. A pyruvate tolerance test (PTT) was performed. Results are expressed as mean ± SEM (n=4-5). “*”, p<0.05, vs. Vehicle group at the same time point. Unpaired Student’s t-test was used to calculate the p value.

FIG. 12E shows that acute treatment with MLN4924 decreased plasma glucose production in male C57BL/6J mice fed a Western diet (WD) for 4 weeks then administered 60 mg/kg MLN4924 as described in FIG. 12A. A glucose tolerance test (GTT) was performed. Results are expressed as mean ± SEM (n=4-5). “*”, p<0.05, vs. Vehicle group at the same time point. Unpaired Student’s t-test was used to calculate the p value.

FIG. 13A shows the results of mice treated with TAS4464. Male C57/BL6J mice were fed a chow diet for 10 weeks then injected with TAS4464 (45 mg/kg) once in the morning. Glucose tolerance test (GTT) was performed 6 h later.

FIG. 13B shows the results of mice treated with TAS4464. Male C57/BL6J mice were fed a chow diet for 10 weeks then injected with TAS4464 (45 mg/kg) once in the morning. Pyruvate tolerance test (PTT) were performed 6 h later. Upper curve is control. Lower curve is TAS4464 treatment.

FIG. 13C shows body weight of mice treated with TAS4464. Male C57BL/6J mice were fed WD for 10 weeks and then injected with TAS4464 (15 mg/kg, SQ) at 5 pm on day 1 and 9 am on day 2. Glucose was measured after 6 h fast on day 2 at 3 pm. Mice were then euthanized and effect of TAS4464 on cullin neddylation was determined in liver. n=5. “*”, p<0.05, vs. Vehicle.

FIG. 13D shows blood glucose of mice treated with TAS4464. Male C57BL/6J mice were fed WD for 10 weeks and then injected with TAS4464 (15 mg/kg, SQ) at 5 pm on day 1 and 9 am on day 2. Glucose was measured after 6 h fast on day 2 at 3 pm. Mice were then euthanized and effect of TAS4464 on cullin neddylation was determined in liver. n=5. “*”, p<0.05, vs. Vehicle.

FIG. 13E is a Western blot showing effects of liver cullin neddylation in mice treated with TAS4464. Male C57BL/6J mice were fed WD for 10 weeks and then injected with TAS4464 (15 mg/kg, SQ) at 5 pm on day 1 and 9 am on day 2. Glucose was measured after 6 h fast on day 2 at 3 pm. Mice were then euthanized and effect of TAS4464 on cullin neddylation was determined in liver. n=5. “*”, p<0.05, vs. Vehicle.

FIG. 14A shows results from male Hepatocyte-specific Cul3 knockout (KO) mice i.v. injected with 1X10¹¹GC/mouse AAV8-TBG-cre (KO) or AAV8-TBG-Null (WT). Mice were fed chow and analyzed 4 weeks later after 6 h fast (9 am-3pm). (n=6-7). “**”, p<0.01. Treated mice have higher IRS 1 and tyrosine (Y)612 phosphorylation in the liver indicating enhanced liver insulin signaling.

FIG. 14B shows densitometry of IRS 1 measured in FIG. 14A.

FIG. 14C shows that WT and Hepatocyte-specific Cul3 KO mice treated as in FIG. 14A show similar body weight.

FIG. 14D shows that Hepatocyte-specific Cul3 KO mice have lower 6-h fasting glucose than WT mice.

FIG. 15A is a Western blot showing that knockdown of Liver Cull by an AAV8-shCul1 (anti-Cul1 short hairpin RNA) lowers blood glucose in mice. Male C57BL/6J mice injected with AAV8-shCul1 or AAV8-Null were fed chow or Western diet for 3 weeks (n=5).

FIG. 15B shows that in mice of FIG. 15A, liver Cull deficiency (by AAV8-shCul1 injection) prevents Western diet-induced hyperglycemia. Blood glucose was measured after 6-h fasting at 3 weeks timepoint “#”, vs. Null+Chow; “*”, vs. Null+WD.

FIG. 15C shows that in mice of FIG. 15A, liver Cull deficiency (by AAV8-shCul1 injection) improves insulin sensitivity. Glucose tolerance test was performed at 3 weeks timepoint. “#”, vs. Null+Chow; “*”, vs. Null+WD.

FIG. 16 shows that treatment with TAS4464 increases serum insulin concentration in mice. Male C57BL6/J mice on chow diet were treated with 45 mg/kg TAS4464 via subcutaneous injection at 9 am. Mice were then fasted for 6 hours and serum insulin was measured. Blood samples were collected at 0 min and 120 min and used for insulin measurement (n=4). Left-hand bars are controls. Right-hand bars are TAS4464 treatments.

FIG. 17A shows that Neddylation inhibition by TAS4464 promotes insulin secretion as indicated by elevated serum c-peptide concentration. Male C57BL6/J mice on Western diet were treated with 15 mg/kg TAS4464 via subcutaneous injection at 9 am. Mice were then fasted for 6 hours and serum c-peptide were measured (n=5).

FIG. 17B shows that Neddylation inhibition by MLN4924 promotes insulin secretion as indicated by elevated serum c-peptide concentration. Male C57BL6/J mice on chow diet were treated with 60 mg/kg MLN4924 via subcutaneous injection at 9 am. Mice were then fasted for 6 hours and serum c-peptide were measured (n=8)

FIG. 18A is a Western blot analysis showing that MLN4924 enhances hepatocyte insulin responsiveness (signaling) by delaying feedback IRS degradation. AML12 cells were serum starved for 16 h. Cells were then pre-treated with MLN4924 for 1 h followed by insulin stimulation for 4 h.

FIG. 18B is a Western blot analysis showing that MLN4924 enhances hepatocyte insulin responsiveness (signaling) by delaying feedback IRS degradation. Primary human hepatocytes cells were serum starved for 16 h. Cells were then pre-treated with MLN4924 for 1 h followed by insulin stimulation for 4 h.

FIG. 18C shows results of a Western blot analysis of IRS protein when different batches of primary human hepatocytes were treated with vehicle (DMSO) or MLN4924 (500 nM) for 8 h. IRS protein in IRS 1 and IRS2 band intensity was normalized to Actin band intensity. n=8-9. “*”, p<0.05 (unpaired t-test), vs. Vehicle.

FIG. 18D is a Western blot analysis of insulin signaling in AML12 cells serum starved for 16 h, then pre-treated with 500 nM MLN4924 for 1 h followed by 100 nM insulin stimulation in time course.

FIG. 18E is a Western blot analysis of AML12 cells serum starved for 16 h then pre-treated with 500 nM MLN4924 or vehicle (DMSO) for 1 h, followed by 100 µg/ml cycloheximide (CHX) and 100 nM insulin treatment. Cells were collected at the indicated time for Western blotting.

FIG. 18F is a Western blot analysis of insulin signaling in AML12 cells serum starved for 16 h then pre-treated with 100 nM rapamycin and 500 nM MLN4924 as indicated for 1 h followed by additional 2 h incubation in the presence or absence of 100 nM insulin. Blots are representative of 2-4 independent experiments.

FIG. 19 shows a Western blot analysis of insulin signaling in primary mouse hepatocytes. MLN4924 enhances hepatocyte insulin responsiveness by delaying feedback IRS degradation (related to FIG. 18A). Cells were serum starved for 16 h then pre-treated with 500 nM MLN4924 for 1 h followed by 100 nM insulin stimulation in time course. Blots are representative of 2 independent experiments.

FIG. 20 is a Western blot analysis showing that MLN4924 enhances hepatocyte insulin responsiveness in cells treated with wortmannin by delaying feedback IRS degradation (related to FIG. 18A). AML12 cells were serum starved for 16 h then pre-treated with 1 µM wortmannin (Wort) and 500 nM MLN4924 as indicated for 1 h followed by additional 2 h incubation in the presence or absence of 100 nM insulin. Blots are representative of 2 independent experiments.

FIG. 21 is a Western blot analysis showing that MLN4924 enhances hepatocyte insulin responsiveness in cells treated with Torin 1 by delaying feedback IRS degradation (related to FIG. 18A). AML12 cells were serum starved for 16 h then pre-treated with 250 nM Torin1 and 500 nM MLN4924 as indicated for 1 h followed by additional 2 h incubation in the presence or absence of 100 nM insulin. Blots are representative of 2 independent experiments.

FIG. 22 shows Western blots which demonstrate that knockdown of Cull or Cul3 increases IRS 1 protein abundance under basal culture condition or upon 6 h treatment of 100 nM insulin+100 µg/ml cycloheximide (CHX). Blots are representative of 2-4 independent experiments.

FIG. 23 shows Cull and Cul3 were simultaneously knocked down in AML12 cells by siRNA. Cells were serum starved for 16 h. Cells were then pre-treated with vehicle (DMSO) or 500 nM MLN4924 for 1 h followed by 100 nM insulin treatment in time course. Blots are representative of 2-4 independent experiments.

FIG. 24 shows Western blots of AML12 cells transfected with IRS1-V5, Myc-Cul1, or Myc-Cul3 as indicated. Immuno-precipitation (IP) was performed with Anti-V5 magnetic beads. Blots are representative of 2-4 independent experiments.

FIG. 25 shows Western blots of IRS protein abundance and AKT phosphorylation in AML12 cells when Cull and Cul3 were simultaneously knocked down by siRNA. Cells were serum starved for 16 h followed by 100 nM insulin treatment in time course in the presence of cycloheximide (CHX). Blots are representative of 2-4 independent experiments.

FIG. 26 is a Western blot which demonstrates that knockdown of Cull or Cul3 increases IRS1 and IRS2 protein abundance. AML12 cells were transfected with control siRNA (siCon) or siRNAs against each of cullin 1, 2, 3, 4A, 4B, 5 and 7. After the AML12 cells were serum-starved for 16 h, cells were treated with 100 nM insulin for 6 h to stimulate IRS protein degradation.

FIG. 27A shows Western blots of protein production in AML12 cells transfected with control siRNA (siCon). After 16 h of serum starvation, cells were pre-treated with 500 nM MLN4926 followed by 100 nM insulin stimulation in time course.

FIG. 27B shows Western blots of protein production in AML12 cells transfected with anti-Cull siRNA (siCul1). After 16 h of serum starvation, cells were pre-treated with 500 nM MLN4926 followed by 100 nM insulin stimulation in time course. An siCon treatment (left lane) is included for comparison to Cull knockdown cells. Knockdown of Cull partially abolishes further MLN4924-mediated insulin sensitization.

FIG. 27C shows Western blots of protein production in AML12 cells transfected with anti-Cul3 siRNA (siCul3). After 16 h of serum starvation, cells were pre-treated with 500 nM MLN4926 followed by 100 nM insulin stimulation in time course. An siCon treatment (left lane) is included for comparison to Cul3 knockdown cells. Knockdown of Cul3 partially abolishes further MLN4924-mediated insulin sensitization.

FIG. 28 is a Western blot analysis which shows that MLN4924 administration does not affect skeletal muscle cullin neddylation or AKT phosphorylation in mice. Male C57BL/6J mice on chow diet were treated with 60 mg/kg MLN6924 via subcutaneous (SQ) injection at 5 pm on day 1 and 9 am on day 2. Mice were fasted from 9 am to 3 pm on day 2 and sacrificed. Effect of MLN4924 on gastrocnemius muscle cullin neddylation and AKT phosphorylation was measured by Western blotting.

FIG. 29 shows that despite lower basal glucose in the MLN4924-treated group, both groups showed similarly decreased plasma glucose (to ~25 mg/dL) in response to insulin after 1 h. Male C7BL/6J mice were first fed WD for 6 weeks, and then treated with 60 mg/kg MLN6924 via subcutaneous (SQ) injection at 5 pm on day 1 and 9 am on day 2. Mice were fasted from 9 am to 3 pm on day 2 and insulin tolerance test was performed with 0.5 U/kg insulin i.p. injection (n=5). All results are expressed as mean ± SEM. “*”, p<0.05, vs. Vehicle group at the same time point. Unpaired Student’s t-test was used to calculate the p value.

FIG. 30 shows that human fatty livers have increased Cullin neddylation. Western blotting of Nedd8 were made using human normal liver and NASH liver total protein lysate (upper panel). Densitometry (lower panel) was determined by ImageJ and expressed as mean ± SEM.

FIG. 31 shows that murine fatty livers have increased Cullin neddylation. Western blotting using liver lysate from male C57BL/6J mice fed chow or Western diet (WD) for 12 weeks. Arrows indicate neddylated cullin. “*”, p<0.05, vs. Normal. Unpaired Student’s t-test was used to calculate the p value.

FIG. 32 shows that MLN4924 does not enhance insulin secretion from INS-1 832/12 cells (a) A Western blot showing MLN4924 treatment of 7 hrs inhibits cullin neddylation, (b) An Insulin secretion assay was performed as described in “Methods”. Insulin secreted into the medium was measured, (c) Total remaining intracellular insulin was measured, (d) The % secreted insulin was the fraction of total medium insulin over the sum of insulin in the medium and cells. Results are from 3 independent experiments and expressed as mean ± SEM. Two-way ANOVA and Tukey post hoc test were used to calculate the p values.

FIG. 33A shows photomicrographs of representative H&E-stained liver samples. Chronic MLN4924 treatment attenuates hepatic steatosis in Western diet-fed mice. Male C57BL/6J mice were fed Chow (C) or Western diet (WD) for 16 weeks. MLN4924 treatment (60 mg/kg, SQ, every other day) was initiated after mice were fed WD for 7 weeks as indicated. Control mice were injected with vehicle. After 16 weeks of feeding, mice were fasted from 9 am to 3 pm and euthanized. Scale bar = 125 µm.

FIG. 33B shows liver weight (LW) of the mice treated in FIG. 33A. Results are expressed as mean ± SEM (n=5-8). Two-way ANOVA and Tukey post hoc test were used to calculate the p values.

FIG. 33C shows liver weight (LW):body weight (BW) ratio of the mice treated in FIG. 33A. Results are expressed as mean ± SEM (n=5-8). Two-way ANOVA and Tukey post hoc test were used to calculate the p values.

FIG. 33D shows liver triglyceride content of the mice treated in FIG. 33A. Results are expressed as mean ± SEM (n=5-8). Two-way ANOVA and Tukey post hoc test were used to calculate the p values.

FIG. 34A shows that chronic MLN4924 treatment does not affect adiposity in Western diet-fed mice. Male C57BL/6J mice were fed Chow (C) or Western diet (WD) for 16 weeks. MLN4924 treatment (60 mg/kg, SQ, every other day) was initiated after mice were fed WD for 7 weeks as indicated. Control mice were injected with vehicle. After 16 weeks of feeding, mice were fasted from 9 am to 3 pm and euthanized and gonadal fat pad weight was measured. Results are expressed as mean ± SEM (n=5-8). Two-way ANOVA and Tukey post hoc test were used to calculate the p values.

FIG. 34B shows that chronic MLN4924 treatment does not affect circulating fatty acids in Western diet-fed mice. Male C57BL/6J mice were fed Chow (C) or Western diet (WD) for 16 weeks. MLN4924 treatment (60 mg/kg, SQ, every other day) was initiated after mice were fed WD for 7 weeks as indicated. Control mice were injected with vehicle. After 16 weeks of feeding, mice were fasted from 9 am to 3 pm and euthanized and serum free fatty acids (FFA) were measured. Results are expressed as mean ± SEM (n=5-8). Two-way ANOVA and Tukey post hoc test were used to calculate the p values.

### DETAILED DESCRIPTION

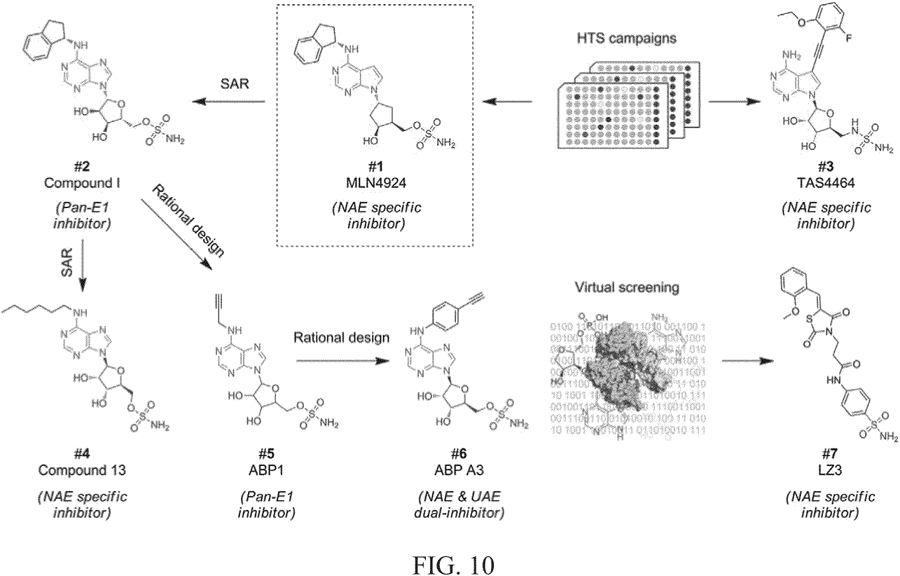

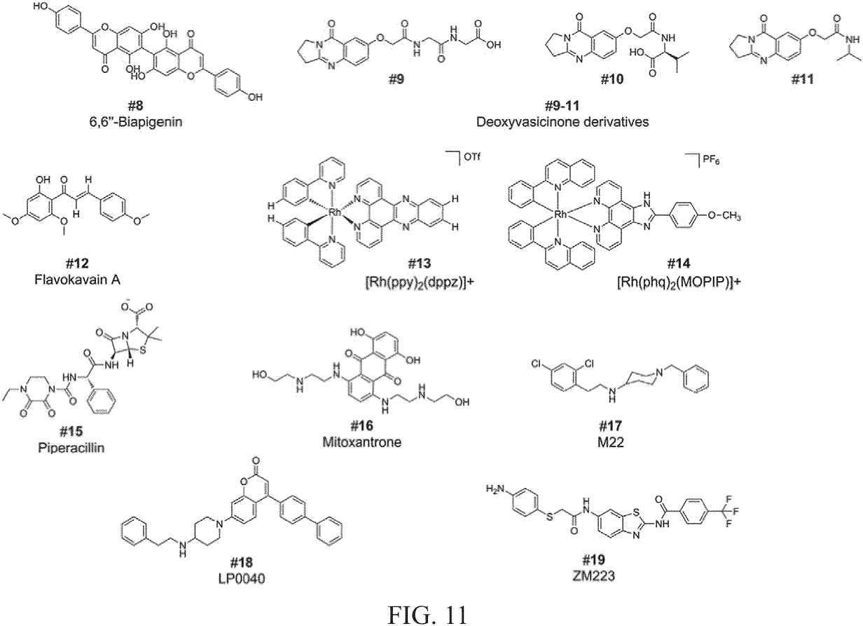

The present disclosure is directed to methods for treating hyperglycemia and/or insulin resistance in patients having such a condition(s) (such as, but not limited to, patients with type-2 diabetes). The Insulin Receptor Substrate proteins (IRS) are a family of cytoplasmic adaptor proteins that transmit signals from the insulin and the Insulin-like growth factor-1 (IGF-1) receptors to elicit a cellular response. Impaired function of IRS (e.g., IRS1, IRS2) contributes significantly to hepatic and muscle insulin resistance, which is a major cause of hyperglycemia in type-2 diabetes. The present disclosure shows that inhibition of CRL, for example by using a neddylation inhibitor (such as, but not limited to, MLN4924 and active derivatives thereof, or TAS4464) to inhibit the NEDD8-Activating Enzyme E1 (NAE) thus decreasing cullin neddylation, reduces IRS protein turnover in liver and muscle cells. This results in an enhanced cellular response to endogenous insulin, thereby achieving rapid tissue insulin sensitization and the lowering of blood glucose concentration (thereby treating hyperglycemia) in individuals with type-2 diabetes. Additional examples of inhibitors of CRL activity, particularly inhibitors of NAE, that can be used in accordance with the methods of the present disclosure, in certain embodiments, include, but are not limited to, Compound 1, Compound 13, ABP1, ABP A3, 1-216, LZ3, 6,6″-Biapigenin, Deoxyvasicinone derivatives, Flavokawain A, [Rh(ppy)₂(dppz)]⁺, [Rh(phq)₂(MOPIP)]⁺, Piperacillin, Mitoxantrone, M22, LP0040, and ZM223 (for specific sources and further information regarding these compounds see FIGS. 10-11 herein, and Table 1 in Yu et al, 2020, op cit.). Other NAE inhibitors which can be used in the various embodiments of the present disclosure are described hereinbelow.

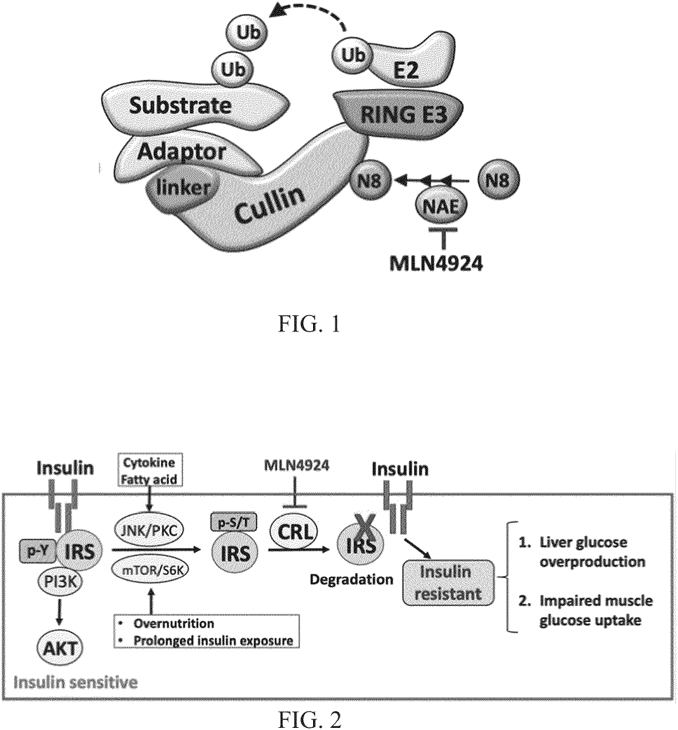

CRLs are a sub-class of ubiquitin ligases. As illustrated in FIG. 2, a CRL is a multi-protein complex containing a cullin scaffold, a RING E3 ligase that recruits ubiquitin-charged E2, and a substrate adaptor which recognizes specific substrates that usually have undergone posttranslational modifications (i.e. phosphorylation). Cullins are a family of hydrophobic scaffold proteins. There are 7 Cullin members (Cul1, Cul2, Cul3, Cul4A, Cul4B, Cul5, and Cul7), each of which serves as the scaffold of a functionally distinct CRL. Mammalian cells express a large number of adaptors in a tissue-specific manner, which further determines the CRL substrate specificity. As noted above, CRLs are activated upon “neddylation” of the cullin component of the CRL, a process in which the small ubiquitin-like protein NEDD8 (N8) is covalently conjugated to a conserved lysine on the cullin scaffold (FIG. 1). Neddylation results in a conformational change in the cullin protein that is a requirement for optimal CRL assembly and function. Neddylation is mediated by a set of NEDD8 E1, E2, and E3 enzymes that sequentially transfer NEDD8 to the cullin in a process that is analogous to the ubiquitin conjugating system. NEDD8-Activating Enzyme E1 (NAE) is the only known NEDD8 E1 enzyme in mammalian cells.

As noted above, impaired function of IRS contributes to hepatic and muscle insulin resistance, which is a major cause of hyperglycemia in type-2 diabetes. FIG. 1 shows how the CRL acts on the IRS, leading to insulin resistance. Upon insulin binding, the cell surface insulin receptor (IR) tyrosine kinase is activated leading to the recruitment of insulin receptor substrate 1 (IRS1) and IRS2 and activation of various downstream signaling pathways. Activation of AKT downstream of insulin signaling is critically involved in mediating a large number of insulin effects, including repression of liver glucose production and stimulation of glucose uptake in skeletal muscle cells and adipocytes. Under obese and diabetic conditions, various nutrient and stress kinases including mTOR/S6K, JNK, PKC are abnormally activated by fatty acids, nutrients, high circulating insulin, and proinflammatory cytokines. These signaling pathways cause serine and threonine phosphorylation at multiple residues on the IRS protein. These protein modifications reduce IRS function and promote IRS protein degradation, for example by CRL, resulting in impaired response of cells to further insulin stimulation. If CRL is inhibited, in accordance with the present disclosure, IRS protein degradation will be reduced, leading to increased uptake of glucose and a reduction in blood glucose, thereby serving to mitigate hyperglycemia.

Before further detailed description of various embodiments of the compositions and methods of use thereof of the present disclosure, it is to be understood that the present disclosure is not limited in application to the details of methods and compositions as set forth in the following description. The description provided herein is intended for purposes of illustration only and is not intended to be construed in a limiting sense. The present disclosure is capable of other embodiments or of being practiced or carried out in various ways. As such, the language used herein is intended to be given the broadest possible scope and meaning, and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that various embodiments of the present disclosure may be practiced without these specific details. In other instances, features which are well known to persons of ordinary skill in the art have not been described in detail to avoid unnecessary complication of the description. It is intended that all alternatives, substitutions, modifications and equivalents apparent to those having ordinary skill in the art are included within the scope of the present disclosure as defined herein. Thus, the examples described below, which include particular embodiments, will serve to illustrate the practice of the present disclosure, it being understood that the particulars shown are by way of example and for purposes of illustrative discussion of particular embodiments only and are presented in the cause of providing what is believed to be a useful and readily understood description of procedures as well as of the principles and conceptual aspects of the inventive concepts. Thus, while the compositions and methods of the present disclosure have been described in terms of particular embodiments, it will be apparent to those of skill in the art that variations may be applied to the compositions and/or methods and in the steps or in the sequence of steps of the method described herein without departing from the concept, spirit, and scope of the inventive concepts disclosed herein.

All patents, published patent applications, and non-patent publications referenced in any portion of this application, including but not limited to U.S. Serial No. 63/076662, and U.S. Pat. Application Publication Nos. US 2013/0116208, US 2016/0160219, US 2016/0250303, US 2017/0066772, US 2017/0107579, US 2018/0162864, and US 2018/0303833, are expressly incorporated herein by reference in their entireties to the same extent as if the individual patent, or published patent application, or non-patent publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, observer error, and combinations thereof, for example. The term “about” or “approximately,” where used herein when referring to a measurable value such as an amount, a temporal duration, and the like, is meant to encompass, for example, variations of ± 20%, or ± 10%, or ± 5%, or ± 1%, or ± 0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 80% of the time, at least 90% of the time, at least 91% of the time, at least 92% of the time, at least 93% of the time, at least 94% of the time, at least 95% of the time, at least 96% of the time, at least 97% of the time, at least 98% of the time, or at least 99% of the time.

As used herein any reference to “one embodiment” or “an embodiment” means that a particular element, feature, composition, structure, or characteristic described in connection with the embodiment is included in at least one embodiment. The appearances of the phrase “in one embodiment” in various places in the specification are not necessarily all referring to the same embodiment. Further, all references to one or more embodiments or examples are to be construed as non-limiting to the claims.

The term “pharmaceutically acceptable” refers to compounds and compositions which are suitable for administration to humans and/or animals without undue adverse side effects such as toxicity, irritation and/or allergic response commensurate with a reasonable benefit/risk ratio.

By “biologically active” is meant the ability to modify the physiological system of an organism without reference to how the active agent has its physiological effects.

As used herein, “pure” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species (e.g., the active agent) is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

The terms “subject” and “patient” are used interchangeably herein and will be understood to refer to a warm-blooded animal, particularly a mammal. Non-limiting examples of animals within the scope and meaning of this term include dogs, cats, rabbits, rats, mice, guinea pigs, chinchillas, hamsters, ferrets, horses, pigs, goats, cattle, sheep, zoo animals, camels, llamas, non-human primates, including Old and New World monkeys and non-human primates (e.g., cynomolgus macaques, chimpanzees, rhesus monkeys, orangutans, and baboons), and humans.

Where used herein the term “active agent” refers to a compound or composition having biological activity. Particular active agents of the present disclosure include, but are not limited to, neddylation inhibitors, and particularly NAE inhibitors, as described elsewhere herein. The term active agent may be used interchangeably herein with the terms “drug,” “therapeutic drug,” “active ingredient,” and “active compound.”

As used herein, the term “active derivative of MLN4924” refers to those compounds, enantiomers, and derivatives of MLN4924, which are derived from MLN4924 and retain all or part of the the NAE inhibitory effect of MLN4924.

“Treatment” refers to therapeutic treatments. “Prevention” refers to prophylactic or preventative treatment measures. The term “treating” refers to administering the composition to a patient for therapeutic purposes, e.g., for reducing hyperglycemia.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “effective amount” refers to an amount of an active agent which is sufficient to exhibit a detectable therapeutic effect without excessive adverse side effects (such as toxicity, irritation and allergic response) commensurate with a reasonable benefit/risk ratio when used in the manner of the inventive concepts. The effective amount for a patient will depend upon the type of patient, the patient’s size and health, the nature and severity of the condition to be treated, the method of administration, the duration of treatment, the nature of concurrent therapy (if any), the specific formulations employed, and the like.

The term “ameliorate” means a detectable or measurable improvement in a subject’s condition, disease or symptom thereof. A detectable or measurable improvement includes a subjective or objective decrease, reduction, inhibition, suppression, limit or control in the occurrence, frequency, severity, progression, or duration of the condition or disease, or an improvement in a symptom or an underlying cause or a consequence of the disease, or a reversal of the disease. A successful treatment outcome can lead to a “therapeutic effect,” or “benefit” of ameliorating, decreasing, reducing, inhibiting, suppressing, limiting, controlling or preventing the occurrence, frequency, severity, progression, or duration of a disease or condition, or consequences of the disease or condition in a subj ect.

A decrease or reduction in worsening, such as stabilizing the condition or disease, is also a successful treatment outcome. A therapeutic benefit therefore need not be complete ablation or reversal of the disease or condition, or any one, most or all adverse symptoms, complications, consequences or underlying causes associated with the disease or condition. Thus, a satisfactory endpoint may be achieved when there is an incremental improvement such as a partial decrease, reduction, inhibition, suppression, limit, control, or prevention in the occurrence, frequency, severity, progression, or duration, or inhibition or reversal of the condition or disease (e.g., stabilizing), over a short or long duration of time (hours, days, weeks, months, etc.). Effectiveness of a method or use, such as a treatment that provides a potential therapeutic benefit or improvement of a condition or disease, can be ascertained by various methods and testing assays.

The active agents of the present disclosure may be present in the pharmaceutical compositions (singly or in combination) at any concentration that allows the pharmaceutical composition to function in accordance with the present disclosure; for example, but not by way of limitation, the compound(s) may be present in a carrier, diluent, or buffer solution in a wt/wt or vol/vol range of the compound: carrier having a lower level selected from 0.00001%, 0.0001%, 0.005%, 0.001%, 0.005%, 0.01%, 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9% and 2.0%; and an upper level selected from 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, and 95%. Non-limiting examples of particular wt/wt or vol/vol ranges include a range of from about 0.0001% to about 95%, a range of from about 0.001% to about 75%; a range of from about 0.005% to about 50%; a range of from about 0.01% to about 40%; a range of from about 0.05% to about 35%; a range of from about 0.1% to about 30%; a range of from about 0.1% to about 25%; a range of from about 0.1% to about 20%; a range of from about 1% to about 15%; a range of from about 2% to about 12%; a range of from about 5% to about 10%; and the like. Any other range that includes a lower level selected from the above-listed lower level concentrations and an upper level selected from the above-listed upper level concentrations also falls within the scope of the present disclosure. Percentages used herein may be weight percentages (wt%) or volume percentages (vol%).

In certain non-limiting embodiments, an effective amount or therapeutic dosage of a pharmaceutical composition of the present disclosure contains, sufficient active agent to deliver from about 0.001 µg/kg to about 100 mg/kg (weight of active agent/body weight of the subject). For example, the composition will deliver about 0.01 µg/kg to about 50 mg/kg, and more particularly about 0.1 µg/kg to about 10 mg/kg, and more particularly about 1 µg/kg to about 1 mg/kg. Practice of a method of the present disclosure may comprise administering to a subject an effective amount of the active agent in any suitable systemic and/or local formulation, in an amount effective to deliver the therapeutic dosage of the active agent. In certain embodiments, an effective dosage may be, in a range of about 1 µg/kg to about 1 mg/kg of the active agent.

Practice of the methods of the present disclosure may comprise administering to a subject therapeutically effective amounts of the active agents in any suitable systemic and/or local formulation, in an amount effective to deliver the dosages listed herein. The dosage can be administered, for example but not by way of limitation, on a one-time basis, or administered at multiple times (for example but not by way of limitation, from one to five times per day, or once or twice per week), or continuously via a venous drip, depending on the desired therapeutic effect. In one non-limiting example of a therapeutic method of the present disclosure, the active agent is provided in an IV infusion in the range of from about 0.01 mg/kg to about 10 mg/kg of body weight once a day.

The term “synergistic” or “synergistic effect” or “synergistic interaction” as used herein refers to a therapeutic combination which is more effective than the additive effects of the two or more single active agents, for example two neddylation inhibitors, or more particularly two NAE inhibitors. A “synergistic ratio” is a ratio of two compounds which results in a synergistic effect. A determination of a synergistic interaction between the active agents described herein may be based on the results obtained from the assays described herein. The results of these assays can be analyzed using the Chou and Talalay combination method (Chou TC, Talalay P. “Quantitative analysis of dose-effect relationships: the combined effects of multiple drugs or enzyme inhibitors.” Adv Enzyme Regul. 1984;22: 27-55) and Dose-Effect Analysis with CalcuSyn software in order to obtain a Combination Index. The combinations provided herein have been evaluated in several assay systems, and the data can be analyzed utilizing a standard program for quantifying synergism, additivism, and antagonism among anticanceragents. An example program is that described by Chou and Talalay, in “New Avenues in Developmental Cancer Chemotherapy,” Academic Press, 1987, Chapter 2. Combination Index values less than 0.9 indicate synergy, values greater than 1.2 indicate antagonism and values between 0.9 to 1.1 indicate additive effects (e.g., see Table 4 below). The combination therapy may provide “synergy” and prove “synergistic”, i.e., the effect achieved when the active agents used together is greater than the sum of the effects that results from using the compounds separately. A synergistic effect may be attained when the active agents are: (1) co-formulated and administered or delivered simultaneously in a combined, unit dosage formulation; (2) delivered in succession (“alternation therapy”) or in parallel as separate formulations; or (3) by some other effective regimen. When delivered in successive administrations, a synergistic effect may be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

The active agents of the combination therapies of the present disclosure may be used conjointly. As used herein the terms “conjointly” or “conjoint administration” refers to any form of administration of two or more different therapeutic compounds (i.e., active agents) such that the second compound is administered while the previously administered therapeutic compound is still effective in the body, whereby the two or more compounds are simultaneously active in the patient, enabling a synergistic interaction of the compounds. For example, the different therapeutic compounds can be administered either in the same formulation, or in separate formulations, either concomitantly (together) or sequentially. When administered sequentially the different compounds may be administered immediately in succession, or separated by a suitable duration of time, as long as the active agents function together in a synergistic manner. In certain embodiments, the different therapeutic compounds can be administered within one hour of each other, within two hours of each other, within 3 hours of each other, within 6 hours of each other, within 12 hours of each other, within 24 hours of each other, within 36 hours of each other, within 48 hours of each other, within 72 hours of each other, or more. Thus an individual who receives such treatment can benefit from a combined effect of the different therapeutic compounds.

The active agents of the present disclosure can be administered to a subject by any of a number of effective routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example via a patch applied to the skin); and topically (for example, as a cream, ointment or spray applied to the skin, or as an eye drop). The compounds may also be formulated for inhalation. In certain embodiments, a compound may be simply dissolved or suspended in sterile water. Oral formulations may be formulated such that the active agent(s) passes through a portion of the digestive system before being released, for example it may not be released until reaching the small intestine, or the colon. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

Tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active agent therein using, for example, hydroxypropylmethyl cellulose in varying proportions to provide the desired release profile, other polymer matrices, liposomes and/or microspheres. They may be sterilized, for example, by filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active agent (s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active agent, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations which are suitable for vaginal administration also include pessaries, tampons, creams, gels, pastes, foams or spray formulations containing such carriers as are known in the art to be appropriate. In certain embodiments, the active agents of the present disclosure can be formulated into suppositories, slow-release formulations, or intrauterine delivery devices (IUDs).

Formulations of the pharmaceutical compositions of the active agent for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Dosage forms for the topical or transdermal administration include powders, sprays, ointments, pastes, creams, lotions, gels, solutions, patches and inhalants. The active agent may be mixed under sterile conditions with a pharmaceutically acceptable carrier, and with any preservatives, buffers, or propellants that may be required. The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane. Transdermal patches have the added advantage of providing controlled delivery of an active agent of the present disclosure to the body. Such dosage forms can be made by dissolving or dispersing the active agent in the proper medium. Absorption enhancers can also be used to increase the flux of the agent across the skin. The rate of such flux can be controlled by either providing a rate controlling membrane or dispersing the agent in a polymer matrix or gel.

Ophthalmic formulations, eye ointments, powders, solutions and the like, are also contemplated as being within the scope of the present disclosure. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A particular route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active agents in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the present disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the active agent then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

As noted, effective amounts of the active agent(s) may be administered orally, in the form of solid or liquid preparations such as capsules, pills, tablets, lozenges, melts, powders, suspensions, solutions, elixirs or emulsions. Solid unit dosage forms can be capsules of the ordinary gelatin type containing, for example, surfactants, lubricants, and inert fillers such as lactose, sucrose, and cornstarch, or the dosage forms can be sustained release preparations. The pharmaceutical composition may contain a solid carrier, such as a gelatin or an adjuvant. The tablet, capsule, and powder may contain from about 0.05 to about 95% of the active substance compound by dry weight. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol, or polyethylene glycol. When administered in liquid form, the pharmaceutical composition particularly contains from about 0.005 to about 95% by weight of the active agent(s). For example, a dose of about 10 mg to about 1000 mg once or twice a day could be administered orally.

In another embodiment, the active agent(s) of the present disclosure can be tableted with conventional tablet bases such as lactose, sucrose, and cornstarch in combination with binders, such as acacia, cornstarch, or gelatin, disintegrating agents such as potato starch or alginic acid, and a lubricant such as stearic acid or magnesium stearate. Liquid preparations are prepared by dissolving the active agents in an aqueous or non-aqueous pharmaceutically acceptable solvent which may also contain suspending agents, sweetening agents, flavoring agents, and preservative agents as are known in the art.

For parenteral administration, for example, the active agent(s) may be dissolved in a physiologically acceptable pharmaceutical carrier and administered as either a solution or a suspension. Illustrative of suitable pharmaceutical carriers are water, saline, dextrose solutions, fructose solutions, ethanol, or oils of animal, vegetative, or synthetic origin. The pharmaceutical carrier may also contain preservatives and buffers as are known in the art.

When an effective amount of the active agent(s) is administered by intravenous, cutaneous, or subcutaneous injection, the compound is particularly in the form of a pyrogen-free, parenterally acceptable aqueous solution or suspension. The preparation of such parenterally acceptable solutions, having due regard to pH, isotonicity, stability, and the like, is well within the skill in the art. A particular pharmaceutical composition for intravenous, cutaneous, or subcutaneous injection may contain, in addition to the active agent, an isotonic vehicle such as Sodium Chloride Injection, Ringer’s Injection, Dextrose Injection, Dextrose and Sodium Chloride Injection, Lactated Ringer’s Injection, or other vehicle as known in the art. The pharmaceutical compositions of the present disclosure may also contain stabilizers, preservatives, buffers, antioxidants, or other additives known to those of skill in the art.

As noted, particular amounts and modes of administration can be determined by one skilled in the art. One skilled in the art of preparing formulations can readily select the proper form and mode of administration, depending upon the particular characteristics of the active agent(s) selected, the condition to be treated, the stage of the condition, and other relevant circumstances using formulation technology known in the art, described, for example, in Remington: The Science and Practice of Pharmacy, 22nd ed.

Additional pharmaceutical methods may be employed to control the duration of action of the active agent(s). Increased half-life and/or controlled release preparations may be achieved through the use of proteins or polymers to conjugate, complex with, and/or absorb the active agent(s) as discussed previously herein. The controlled delivery and/or increased half-life may be achieved by selecting appropriate macromolecules (for example but not by way of limitation, polysaccharides, polyesters, polyamino acids, homopolymers, polyvinyl pyrrolidone, ethylenevinylacetate, methylcellulose, or carboxymethylcellulose, and acrylamides such as N-(2-hydroxypropyl) methacrylamide), and the appropriate concentration of macromolecules as well as the methods of incorporation, in order to control release.

Another possible method useful in controlling the duration of action of the active agent(s) by controlled release preparations and half-life is incorporation of the active agents or their functional derivatives into particles of a polymeric material such as polyesters, polyamides, polyamino acids, hydrogels, poly(lactic acid), ethylene vinylacetate copolymers, copolymer micelles of, for example, polyethylene glycol (PEG) and poly(1-aspartamide).

It is also possible to entrap the active agent(s) in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles, and nanocapsules), or in macroemulsions. Such techniques are well known to persons having ordinary skill in the art.

When the active agents are to be used as an injectable material, they can be formulated into a conventional injectable carrier. Suitable carriers include biocompatible and pharmaceutically acceptable phosphate buffered saline solutions, which are particularly isotonic.

For reconstitution of a lyophilized product in accordance with the present disclosure, one may employ a sterile diluent, which may contain materials generally recognized for approximating physiological conditions and/or as required by governmental regulation. In this respect, the sterile diluent may contain a buffering agent to obtain a physiologically acceptable pH, such as sodium chloride, saline, phosphate-buffered saline, and/or other substances which are physiologically acceptable and/or safe for use. In general, the material for intravenous injection in humans should conform to regulations established by the Food and Drug Administration, which are available to those in the field. The pharmaceutical composition may also be in the form of an aqueous solution containing many of the same substances as described above for the reconstitution of a lyophilized product.

The active agent(s) can also be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, tauric acid, maleic acid, and fumaric acid, or by reaction with an inorganic base such as sodium hydroxide, ammonium hydroxide, potassium hydroxide, and organic bases such as mono-, di-, trialkyl and aryl amines, and substituted ethanolamines.

In certain embodiments, the present disclosure includes an active agent composition wherein at least one of the active agents is coupled (e.g., by covalent bond) directly or indirectly (via a linker molecule) to a carrier molecule.

As noted above, the present disclosure is directed to a method of treating or mitigating the effects of type-2 diabetes including, but not limited to, hyperglycemia and insulin resistance. The method comprises administering to the subject an amount of at least one active agent (e.g., a neddylation inhibitor) which is effective in inhibiting a CRL, for example by inhibiting the neddylation of the cullin protein of CRL, and thereby reducing IRS protein degradation.

According to at least some embodiments of the present disclosure, the neddylation inhibitor is a NEDD8-activating enzyme (NAE) inhibitor. According to certain embodiments, the NAE inhibitor may be a “small molecule” compound or a synthetic nucleic acid. Non-limiting examples of “small molecule” NAE inhibitors include, MLN4924 (Pevonedistat) and an analog or derivative thereof, TAS4464 (CAS No. 1848959-10-3), 6,6″-Biapigenin, cyclometalated rhodium (III) complexes such as but not limited to [Rh(ppy)₂(dppz)]⁺ and [Rh(phq)₂(MOPIP)]⁺, and Flavokawain A. Others include but are not limited to Compound 1, Compound 13, ABP1, ABP A3, LZ3, Deoxyvasicinone derivatives, Piperacillin, Mitoxantrone, M22, LP0040, and ZM223 (e.g., see FIGS. 10-11). The synthetic nucleic acid neddylation inhibitors may be a small interference ribonucleic acid (siRNA), a small hairpin ribonucleic acid (shRNA), or a micro-ribonucleic acids (miRNA) that inhibits expression of UBA3, the catalytic subunit of NAE. According to one working example of the present disclosure, the synthetic nucleic acid is an siRNA.

Other NAE inhibitors that can be used in accordance with the present disclosure include, but are not limited to, NAE inhibitors disclosed in the following U.S. Pat. Publications: 2013/0116208 (e.g., ((1S,2S,4R)-4-(4-((1S)-2,3-dihydro-1H-inden-1-ylamino)-7H-pyrrolo[2,3-d]pyrimidin-7-yl)-2-hydroxycyclopentyl) methyl sulfamate (“MLN4924”) or {(1S,2S,4R)-4-[(6-{[(1R,2S)-5-chloro-2-methoxy-2,3-dihydro-1H-inden-1-yl]- amino}pyrimidin-4-yl)oxy]-2-hydroxycyclopentyl}methyl sulfamate (“I-216”), 2016/0160219 (e.g., siRNAs and shRNAs of Tables 1-3), 2016/0250303, 2017/0066772 (e.g., 4-amino-5-[2-(2,6-difluoro phenyl)ethynyl]-7-[(2R,3R,4S,5R)-3,4-dihydroxy-5-[(sulfamoylamino)methyl]- tetrahydrofuran-2-yl]pyrrolo[2,3-d]pyrimidine; 4-amino-5-[2-(4-amino-2,6-difluoro-phenyl)ethynyl]-7-[(2R,3R,4S,5R)-3,4-dihydroxy-5-[(sulfamoylamino)methyl]tetrahydrofuran-2-yl]pyrrolo[2,3-d]pyrimidine; 4-amino-5-[2-[2,6-difluoro-4-(methylamino)phenyl]ethynyl]-7-[(2R,3-R,4S,5R)-3,4-dihydroxy-5-[(sulfamoylamino)methyl]tetrahydrofuran-2-yl]pyrrolo[2,3-d]pyrimidine; 4-amino-5-[2-[2,6-difluoro-4-[(3R)-3-fluoropyrrolidin-1-yl]phenyl]ethynyl]-7-[(2R,3R,4S,5R)-3,4-dihydroxy-5-[(sulfamoylamino)methyl]tetrahydrofuran-2-yl]pyrrolo[2,3-d]pyrimidine; 4-amino-7-[(2R,3R,4S,5R)-3,4-dihydroxy-5-[(sulfamoylamino)methyl]tetrahyd- rofuran-2-yl]-5-[2-(2-ethoxy-4,6-difluoro-phenyl)ethynyl]pyrrolo[2,3-d]pyr- imidine; 4-amino-5-[2-[2,6-difluoro-4-(3-hydroxyazetidin-1-yl)phenyl]ethynyl]-7-[(2R,3R,4S,5R)-3,4-dihydroxy-5-[(sulfamoylamino) methyl]tetrahydrofuran-2-yl]pyrrolo[2,3-d]pyrimidine; 4 amino-5-[2-[4-(azetidin-1-yl)-2,6-difluorophenyl]ethynyl]-7-[(2R,3R,4S,5-R)-3,4-dihydroxy-5[(sulfamoylamino) methyl]tetrahydrofuran-2-yl] pyrrolo[2,-3-d]pyrimidine; 4-amino-7-[(2R,3R,4S,5R)-3,4-dihydroxy-5-[(sulfamoylamino)methyl]tetrahydrofuran-2-yl]-5-[2-(2-ethoxy-6-fluorophenyl)ethynyl]pyrrolo[2,3-d]pyrimidine; 4-amino-7-[(2R,3R,4S,5R)-3,4-dihydroxy-5-[(sulfamoylamino)methyl]tetrahydrofuran-2-yl]-5-[2-(2-fluoro-6-propoxyphenyl)ethynyl]pyrrolo[2,3-d]pyrimidine; 8-[2-[4-amino-7-[(2R,3R,4S,5R)-3,4-dihydroxy-5-[(sulfamoylamino)- methyl]tetrahydrofuran-2-yl]pyrrolo[2,3-d]pyrimidin-5-yl]ethynyl]-7-fluoro- -4-methyl-2,3-dihydro-1,4-benzoxazine; 4-amino-7-[(2R,3R,4S,5R)-3,4-dihydroxy-5-[(sulfamoylamino)methyl]tetrahydrofuran-2-yl]-5-[2-(2-ethylsulfanyl-6-fluorophenyl)ethynyl]pyrrolo[2,3-d]- pyrimidine; 4-amino-5-[2-[2-(cyclopropyl methoxy)-6-fluorophenyl]ethynyl]-7-[(2R,3R,4S,5R)-3,4-dihydroxy-5-[(sulf- amoylamino)methyl]tetrahydrofuran-2-yl]pyrrolo[2,3-d]pyrimidine; 4-amino-7-[(1R,2S,3R,4R)-2,3-dihydroxy-4-[(sulfamoylamino)methyl]cyclopentyl]-5-[2-(2-fluoro-6-methylsulfanylphenyl)ethynyl]pyrrolo[2,3-d]pyrimidine; 8-[2-[4-amino-7-[(1R,2S,3R,4R)-2,3-dihydroxy-4-[(sulfamoylamino)methyl]-cyclopentyl]pyrrolo[2,3-d]pyrimidin-5-yl]ethynyl]-7-fluoro-4-methyl-2,3-dihydro-1,4-benzoxazine; 4-amino-7-[(1R,4R, 5 S)-4,5-dihydroxy-3-[(sulfamoylamino)methyl]cyclopent-2-en-1-yl]-5-[2-(2-ethoxy-6-fluorophenyl)ethynyl]pyrrolo[2,3-d]pyrimidine; and 4-amino-7-[(1R,4R,5S)-4,5-dihydroxy-3-[(sulfamoylamino)methyl]cyclopent-2-en-1-yl]-5-[2-(2-fluoro-6-methylsulfanyl-phenyl) ethynyl]pyrrolo[2,3-d- ]pyrimidine; and salts of these compounds), 2017/0107579, 2018/0162864 (e.g., 4-amino-5-[2-(2,6-difluoro phenyl)ethynyl]-7-[(2R,3R,4S,5R)-3,4-dihydroxy-5-[(sulfamoylamino)methyl]- tetrahydrofuran-2-yl]pyrrolo[2,3-d]pyrimidine; 4-amino-5-[2-(4-amino-2,6-difluoro-phenyl) ethynyl]-7-[(2R,3R,4S,5R)-3,4-dihydroxy-5-[(sulfamoylamino)methyl] tetrahydrofuran-2-yl]pyrrolo[2,3-d]pyrimidine; 4-amino-5-[2-[2,6-difluoro-4-(methylamino)phenyl]ethynyl]-7-[(2R,3R,4S,5R)-3,4-dihydroxy-5-[(sulfamoylamino)methyl]tetrahydrofuran-2-yl]pyrrolo[2,3 -d]pyrimidine; 4-amino-5-[2-[2,6-difluoro-4-[(3R)-3-fluoro pyrrolidin-1-yl]phenyl]ethynyl]-7-[(2R,3R,4S,5R)-3,4-dihydroxy-5-[(sulfamoylamino)methyl]tetrahydrofuran-2-yl]pyrrolo[2,3-d]pyrimidine; 4-[4-[2-[4-amino-7-[(2R,3R,4S,5R)-3,4-dihydroxy-5-[(sulfamoylamino)methyl]tetrahydrofuran-2-yl]pyrrolo[2,3-d]pyrimidin-5-yl]ethynyl]-3-ethoxy-5-fluoro-phenyl]morpholine; 4-amino-7-[(2R,3R,4S,5R)-3,4-dihydroxy-5-[(sulfamoylamino)methyl]tetrahydrofuran-2-yl]-5-[2-(2-ethoxy-4,6-difluorophenyl)ethynyl]pyrrolo[2,3-d]pyrimidine; 4-[4-[2-[4-amino-7-[(2R,3R,4S,5R)-3,4-dihydroxy-5-[(sulfamoylamino)methyl]tetrahydrofuran-2-yl]pyrrolo[2,3-d]pyrimidin-5-yl]ethynyl]-3,5-difluorophenyl]thio morpholine; 4-amino-5-[2-[2,6-difluoro-4-(3-hydroxy azetidin-1-yl)phenyl]ethynyl]-7-[(2R,3R,4S,5R)-3,4-dihydroxy-5-[(sulfamoylamino)methyl] tetrahydrofuran-2-yl]pyrrolo[2,3-d]pyrimidine; 4-amino-5-[2-[4-(azetidin-1-yl)-2,6-difluoro-phenyl]ethynyl]-7-[(1R,2S,3R,4R)-2,3-dihydroxy-4-[(sulfamoylamino)methyl] cyclopentyl]pyrrolo[2,3-d]pyrimidine; 4-amino-7-[(2R,3R,4S,5R)-3,4-dihydroxy-5-[(sulfamoylamino)methyl] tetrahydrofuran-2-yl]-5-[2-(2-ethoxy-6-fluoro-phenyl)ethynyl] pyrrolo[2,3-d]pyrimidine; 4-amino-7-[(2R,3R,4S,5R)-3,4-dihydroxy-5-[(sulfamoylamino) methyl] tetrahydrofuran-2-yl]-5-[2-(2-fluoro-6-propoxyphenyl)ethynyl]pyrrolo[2,3-d] pyrimidine; 4-amino-7-[(2R,3R,4S,5R)-3,4-dihydroxy-5-[(sulfamoylamino)methyl] tetrahydrofuran-2-yl] -5-[2-[2-fluoro-6-(2,2,2-trifluoroethoxy)phenyl] ethynyl]pyrrolo[2,3-d] pyrimidine; 8-[2-[4-amino-7-[(2R,3R,4S,5R)-3,4-dihydroxy-5-[(sulfamoylamino)methyl] tetrahydrofuran-2-yl]pyrrolo[2,3-d]pyrimidin-5-yl] ethynyl]-7-fluoro-4-methyl-2,3-dihydro-1,4-benzoxazine; 4-amino-7-[(2R,3R,4S,5R)-3,4-dihydroxy-5-[(sulfamoylamino)methyl] tetrahydrofuran-2-yl]-5-[2-(2-ethylsulfanyl-6-fluorophenyl)ethynyl]pyrrolo[2,3-d]-pyrimidine; 4-amino-5-[2-[2-(cyclopropyl methoxy)-6-fluorophenyl]ethynyl]-7-[(2R,3R,4S,5R)-3,4-dihydroxy-5-[(sulfamoylamino)methyl] tetrahydrofuran-2-yl]pyrrolo[2,3-d]pyrimidine; 4-amino-7-[(1R,2S,3R,4R)-2,3-dihydroxy-4-[(sulfamoylamino)methyl] cyclopentyl]-5-[2-(2-ethoxy-6-fluorophenyl) ethynyl]pyrrolo[2,3-d]pyrimidine; 4-amino-7-[(1R,2S,3R,4R)-2,3-dihydroxy-4-[(sulfamoylamino)methyl]cyclopentyl]-5-[2-(2-fluoro-6-methylsulfanyl-phenyl)ethynyl] pyrrolo[2,3-d]pyrimidine; 8-[2-[4-amino-7-[(1R,2S,3R,4R)-2,3-dihydroxy-4-[(sulfamoylamino)methyl ]cyclopentyl]pyrrolo[2,3-d]pyrimidin-5-yl]ethynyl]-7-fluoro-4-methyl-2,3-dihydro-1,4-benzoxazine; 4-amino-7-[(1R,4R,5S)-4,5-dihydroxy-3 [(sulfamoylamino)methyl] cyclopent-2-en-1-yl]-5-[2-(2-ethoxy-6-fluorophenyl)ethynyl]pyrrolo[2,3-d]pyrimidine; 4-amino-7-[(1R,4R, 5 S)-4, 5-dihydroxy-3-[(sulfamoylamino)methyl]cyclopent-2-en-1-yl]-5-[2-(2-fluoro-6-methylsulfanylphenyl)ethynyl]pyrrolo[2,- 3-d]pyrimidine; [(2R,3S,4R,5R)-5-[4-amino-5-[2-(2,6-difluoro phenyl)ethynyl]pyrrolo[2,3-d]pyrimidin-7-yl]-3,4-dihydroxytetrahydrofuran-2-yl] methyl sulfamate; 4-amino-5-[2-(2,6-difluorophenyl)ethynyl]-7-[(2R,3R,4S,5R)-3,4-dihydroxy-5-[(sulfamoylamino)methyl] tetrahydrofuran-2-yl]pyrrolo[2,3-d]pyrimidine; 4-amino-5-[2-(4-amino-2,6-difluoro-phenyl) ethynyl]-7-[(2R,3R,4S,5R)-3,4-dihydroxy-5-[(sulfamoylamino)methyl]tetrahydrofuran-2-yl]pyrrolo[2,3-d]pyrimidine; 4-amino-5-[2-[2,6-difluoro-4-(methylamino)phenyl]ethynyl]-7-[(2R,3R,4S,5R)-3,4-dihydroxy-5-[(sulfamoylamino)methyl] tetrahydrofuran-2-yl]pyrrolo[2,3-d]pyrimidine; 4-amino-5-[2-[2,6-difluoro-4-[(3R)-3-fluoro pyrrolidin-1-yl]phenyl]ethynyl]-7-[(2R,3R,4S,5R)-3,4-dihydroxy-5-[(sulfamoylamino)methyl]tetrahydrofuran-2-yl]pyrrolo[2,3-d]pyrimidine; 4-amino-7-[(2R,3R,4S,5R)-3,4-dihydroxy-5-[(sulfamoylamino)methyl]tetrahydrofuran-2-yl]-5-[2-(2-ethoxy-4,6-difluorophenyl)ethynyl]pyrrolo[2,3-d]pyrimidine; 4-amino-5-[2-[2,6-difluoro-4-(3-hydroxy azetidin-1-yl)phenyl]ethynyl]-7-[(2R,3R,4S,5R)-3,4-dihydroxy-5-[(sulfamoylamino)methyl]tetrahydrofuran-2-yl]pyrrolo[2,3-d]pyrimidine; 4-amino-5-[2-[4-(azetidin-1-yl)-2,6-difluorophenyl]ethynyl]-7-[(1R,2S,3R,4R)-2,3-dihydroxy-4-[(sulfamoylamino)methyl] cyclopentyl]pyrrolo[2,3-d]pyrimidine; 4-amino-7-[(2R,3R,4S,5R)-3,4-dihydroxy-5-[(sulfamoylamino)methyl]tetrahyd- rofuran-2-yl]-5-[2-(2-ethoxy-6-fluoro-phenyl)ethynyl] pyrrolo[2,3-d]pyrimidine; 4-amino-7-[(2R,3R,4S,5R)-3,4-dihydroxy-5[(sulfamoylamino)methyl] tetrahydrofuran-2-yl]-5-[2-(2-fluoro-6-propoxy-phenyl)ethynyl]pyrrolo[2,3-d]pyrimidine; 8-[2-[4-amino-7-[(2R,3R,4S,5R)-3,4-dihydroxy-5-[(sulfamoylamino)methyl]tetrahydrofuran-2-yl]pyrrolo[2,3-d]pyrimidin-5-yl]ethynyl]-7-fluoro-4-methyl-2,3-dihydro-1,4-benzoxazine; 4-amino-7-[(2R,3R,4S,5R)-3,4-dihydroxy-5-[(sulfamoylamino)methyl]tetrahydrofuran-2-yl]-5-[2-(2-ethylsulfanyl-6-fluorphenyl)ethynyl]pyrrolo[2,3-d]pyrimidine; 4-amino-5-[2-[2-(cyclopropyl methoxy)-6-fluorophenyl]ethynyl]-7-[(2R,3R,4S,5R)-3,4-dihydroxy-5-[(sulf-amoylamino) methyl]tetrahydrofuran-2-yl]pyrrolo[2,3-d]pyrimidine; 4-amino-7-[(1R,2S,3R,4R)-2,3-dihydroxy-4-[(sulfamoylamino)methyl]cyclopentyl]-5-[2-(2-fluoro-6-methylsulfanyl-phenyl)ethynyl] pyrrolo[2,3-d]pyrimidine; 8-[2-[4-amino-7-[(1R,2S,3R,4R)-2,3-dihydroxy-4-[(sulfamoylamino)methyl]cyclopentyl]pyrrolo[2,3-d]pyrimidin-5-yl] ethynyl]-7-fluoro-4-methyl-2,3-dihydro-1,4-benzoxazine; 4-amino-7-[(1R,4R,5S)-4,5-dihydroxy-3[(sulfamoylamino)methyl] cyclopent-2-en-1-yl]-5-[2-(2-ethoxy-6-fluoro-phenyl)ethynyl] pyrrolo[2,3-d]pyrimidine; 4-amino-7-[(1R,4R,5S)-4,5-dihydroxy-3-[(sulfamoylamino)methyl] cyclopent-2-en-1-yl]-5-[2-(2-fluoro-6-methylsulfanyl-phenyl)ethynyl]pyrrolo[2,3-d] pyrimidine; and salts of these compounds), and 2018/0303833 (e.g., siRNAs, shRNAs, or miRNAs described in Table 1, which target the catalytic subunit UBA3 of NAE), each of which is expressly incorporated herein by reference in its entirety.

According to some embodiments of the present disclosure, the active agent, e.g., neddylation inhibitor, is administered to the subject daily. The active agent may be administrated by a route selected from the group consisting of oral, enteral, nasal, topical, transmucosal, and parenteral administration, in which the parenteral administration is any of subcutaneous, intradermal, intramuscular, intraarterial, intravenous, intraspinal, intrathecal or intraperitoneal injection.

According to certain embodiments of the present disclosure, the method comprises administering to the subject an effective amount of a neddylation inhibitor. In general, the neddylation inhibitor may be any antagonist that inhibits the neddylation pathway. According to certain embodiments of the present disclosure, the neddylation inhibitor may specifically inhibit the activity of NAE. The NAE inhibitor may be a compound or a synthetic nucleic acid. In a particular embodiment of the present disclosure, the neddylation inhibitor is MLN4924, or an analog, derivative, or salt thereof, in which MLN4924 has the chemical formula (1S,2S,4R) -4-(4-((1S)-2,3-Dihydro-1H-inden-1-ylamino)-7H-pyrrolo (2,3-d)pyrimidin-7-yl) -2-hydroxycyclo pentyl) methyl sulphamate. In another particular embodiment, the neddylation inhibitor is TAS4464 (CAS No. 1848959-10-3), or analog, derivative, or salt thereof.

The synthetic nucleic acid NAE inhibitor may be an siRNA, an shRNA, or an miRNA. According to some embodiments of the present disclosure, the synthetic nucleic acid is the siRNA. For example, the siRNA may have a sense strand (serving as the passenger strand) and an anti-sense strand (serving as the guide strand to silence the gene expression). Examples of such nucleic acids include, but are not limited to, those shown in U. S. Pat. Publication 2018/0303833, the entirety of which is hereby explicitly incorporated herein by reference.

As can be appreciated by persons having ordinary skill in the art, the silencing or inhibition of mRNA translation can be achieved by nucleotide molecules other than siRNAs. For instance, shRNA is an RNA molecule that contains sense and anti-sense sequences connected by a short spacer of nucleotides that enables the molecule to form a loop structure. Alternatively, the synthetic nucleic acid is provided in the form of an miRNA or a precursor (e.g., pri-miRNA or pre-miRNA) thereof. Alternatively, the synthetic nucleic acid can be any double- or single-stranded antisense oligonucleotide comprising a sequence which binds to an inhibits expression of

According to some embodiments of the present disclosure, the amount of the present neddylation inhibitor suitable for use in a human subject may be in the range of about 0.01-100 mg/Kg/day, such as 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 mg/kg body weight per day for human; and more particularly in a range of 0.1-10 mg/kg body weigh per day; or 0.1-2.5 mg/kg body weigh per day.

Various embodiments of the present disclosure will be more readily understood by reference to the following examples and description, which as noted above are included merely for purposes of illustration of certain aspects and embodiments of the present disclosure, and are not intended to be limiting. The following detailed examples and methods which describe various compositions of the present disclosure and are to be construed, as noted above, only as illustrative, and not limitations of the disclosure in any way whatsoever.

### Experimental I

### Methods

### Mice and Diet

Male C57BL/6J mice were purchased from the Jackson Laboratory (Bar Harbor, ME). Mice were fed a regular chow (C) diet or a Western diet (WD) that contains 42% fat calories and 0.2% cholesterol content (TD. 88137, Envigo Inc. Indianapolis, IN).

### MLN4924 and TAS4464 Preparation and Treatment

MLN4924 and TAS4464 were purchased from MedChemExpress (MCE, Monmouth Junction, NJ). For cell treatment, MLN4924 and TAS4464 were dissolved in DMSO. Cells were serum starved overnight and then treated with MLN4924, TAS4464 and/or insulin as indicated. For injection in mice, MLN4924 and TAS4464 were first dissolved in DMSO, which was further suspended in 10% (2-hydroxypropyl)-β-cyclodextrin (Sigma, St. Louis, MO). The final solution contains 1% DMSO. The subcutaneous injection volume is 200 µl/mouse.

### Glucose Tolerance Test

On the day of testing, mice were fasted for 6 h (from 9 am to 3 pm). Mice were then given 2 g/kg D-glucose via IP injection. Blood glucose was measured with an UltraTouch glucometer in time course over a 120-minute period.

### Pyruvate Tolerance Test

On the day of testing, mice were fasted for 6 h (from 9am to 3 pm). Mice were then given 2 g/kg sodium pyruvate via IP injection. Blood glucose was measured with an UltraTouch glucometer in time course over a 120-minute period.

### Lipid Extraction and Measurement

Lipids were extracted in a mixture of chloroform: methanol (2:1; v: v), dried under nitrogen, and resuspended in isopropanol containing 1% triton X-100. Total cholesterol, free cholesterol and TG were measured with assay kits following the manufacturer’s instruction. Total cholesterol assay kit and TG assay kit were purchased from Pointe Scientific (Canton, MI).

### Serum Parameters

Aspartate aminotransferase (AST) and alanine aminotransferase (ALT) assay kits were purchased from Pointe Scientific (Canton, MI). Free fatty acid assay kit was purchased from Biovision Inc. (Milpitas, CA). Insulin ELISA kit was purchased from Crystal Chem Inc. (Elk Grove Village, IL).

### Western Blotting

Protein lysates were prepared in RIPA buffer containing 1% SDS and protease inhibitors on ice for 1 h followed by brief sonication. After centrifugation, supernatant was used for SDS-PAGE and immunoblotting.

### Real-time PCR

Total RNA was purified by Trizol (Sigma-Aldrich, St. Louis, MO). Reverse transcription was performed with Oligo dT primer and SuperScript III reverse transcriptase (Thermo Fisher Scientific, Grand Island, NY). Real-time PCR was performed with iQ SYBR Green Supermix (Biorad, Hercules, CA). Relative mRNA expression was calculated using the comparative CT (Ct) method and expressed as 2 ^{-ΔΔCt} with the control group arbitrarily set as “1”.

### Transfection of siRNA

SMARTPool siRNA against Cullins and SMARTPool control siRNA were purchased from Dharmacon Inc. (Lafayette, CO). Cells were transfected with 30 nM siRNA with lipofectamine RNAiMAX (ThermoFisher Scientific, Waltham, MA) according to the manufacturer’s instruction. Further treatments of insulin were carried out 48 h after siRNA transfection.

### Glucose Uptake Assay

C2C12 myoblast cells were cultured in DMEM medium supplemented with 10% FBS. When cells grow to 60% confluency, differentiation into myocytes was induced by culturing cells in DMEM supplemented with 2% horse serum for 4-6 days. Cells were then serum starved overnight and treated with 500 nM MLN4924 or DMSO for 6 h and then stimulated with 100 nM insulin for 1 hour as indicated. Glucose uptake was measured with a Glucose uptake Glo^{tm} assay kit that was purchased from Promega (Madison, WI).

### Results and Discussion

### Fatty Livers Show CRL Hyper-neddylation and Lower IRS Protein Abundance

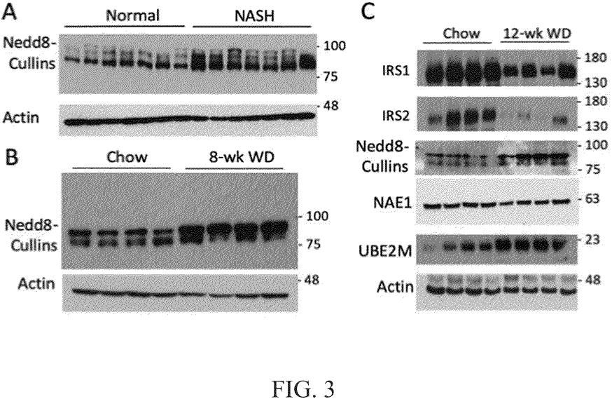

Increased neddylated proteins were detected in human non-alcoholic steatohepatitis (NASH) livers and murine steatosis livers induced by 8 weeks feeding of WD to mice (FIGS. 3a-b). Major neddylated protein bands corresponded to cullin proteins between 75 and 100 KDa, consistent with cullin members being the major physiological neddylation targets in mammalian cells. The causes of increased neddylation in fatty livers are not clear. NAE1 was not increased, but the major Nedd8 E2 enzyme UBE2M was significantly increased in fatty liver (FIG. 3c). Hepatic IRS1 and IRS2 were reduced in steatosis liver, which negatively correlated with CRL neddylation (FIG. 3c).

### MLN4924 Inhibits Hepatic CRL Neddylation Leading to Increased Hepatic Insulin Signaling and Lowered Glucose Production in Mice

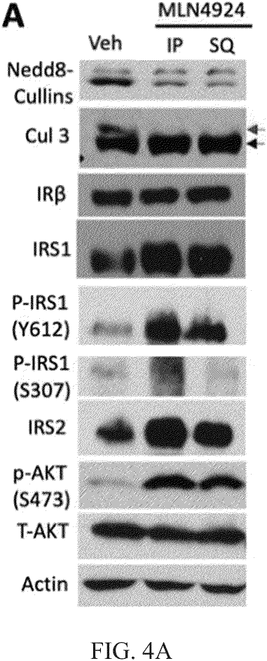

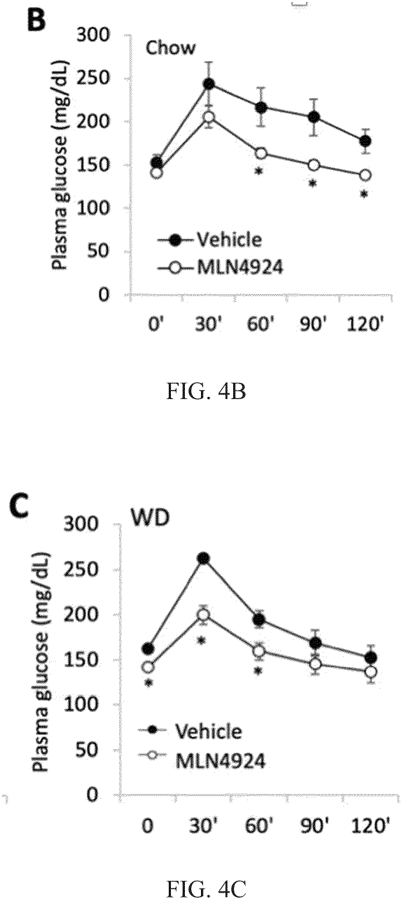

To determine the effect of CRL inhibition on hepatic insulin signaling and hepatic glucose production, we administered MLN4924 to chow-fed mice twice via either intraperitoneal (IP) or subcutaneous (SQ) injection (5 pm on day 1 and 9 am on day 2), which effectively reduced hepatic CRL neddylation as reflected by reduced hepatic neddylated cullins bands and reduced neddylated Cul3 (FIG. 4A). MLN4924 rapidly increased hepatic IRS1 and IRS2, but not insulin receptor (IR), and increased Y612 phosphorylated IRS1 which correlated with higher phosphorylated AKT (FIG. 4B), indicating increased insulin signaling activation. Pyruvate tolerance test was used to determine hepatic glucose production in 4-week C-fed mice and 4-week WD-fed mice, which revealed that blood glucose excursion was significantly lower in MLN4924-treated groups (FIGS. 4B-4C, respectively), suggesting MLN4924 inhibited hepatic glucose production.

### MLN4924 Effects on Blood Glucose in Obese and Insulin Resistant Mice

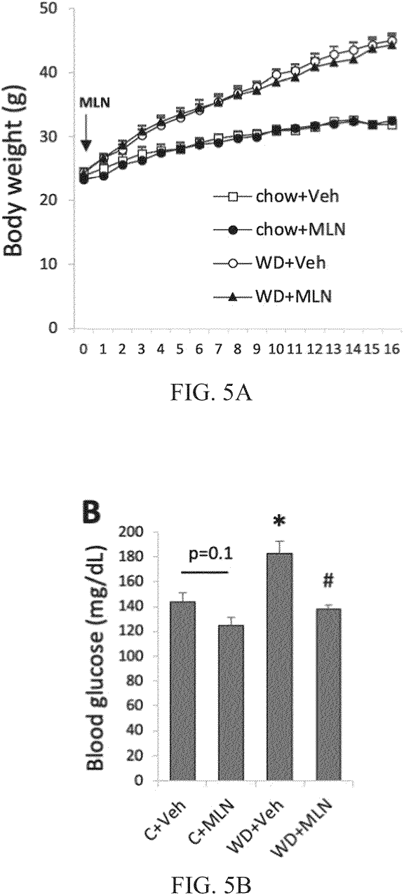

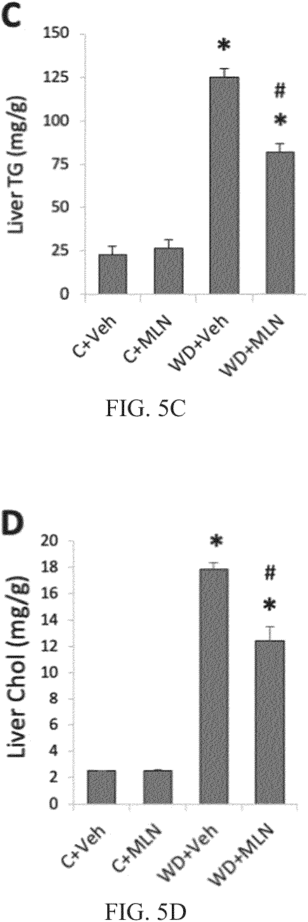

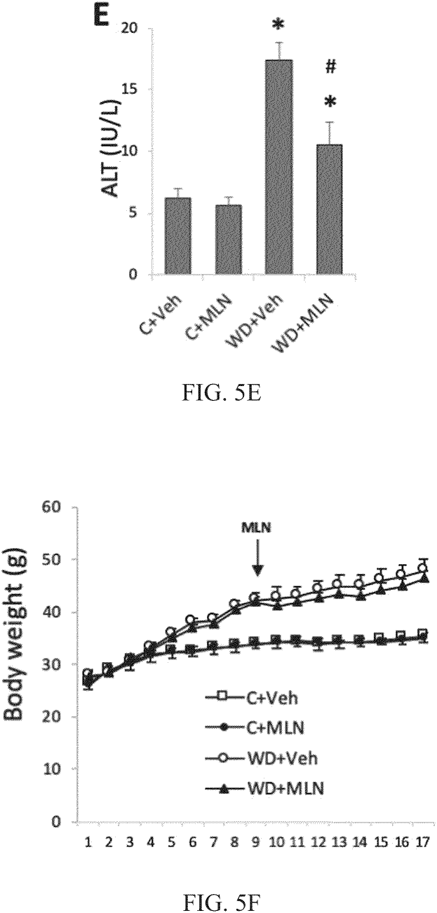

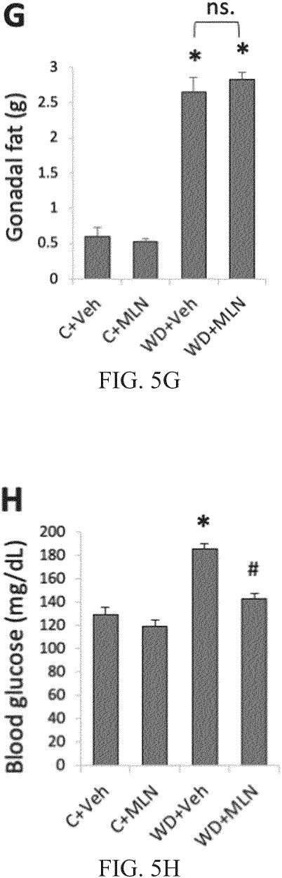

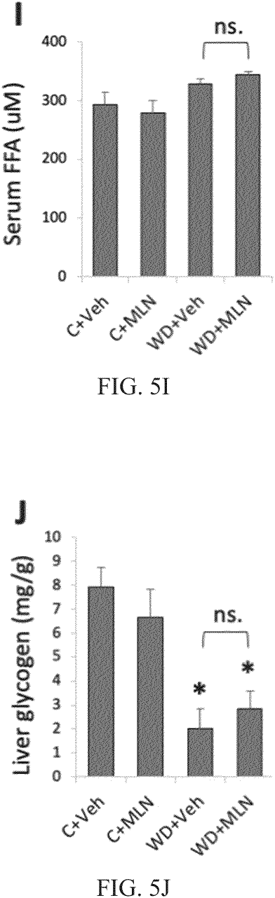

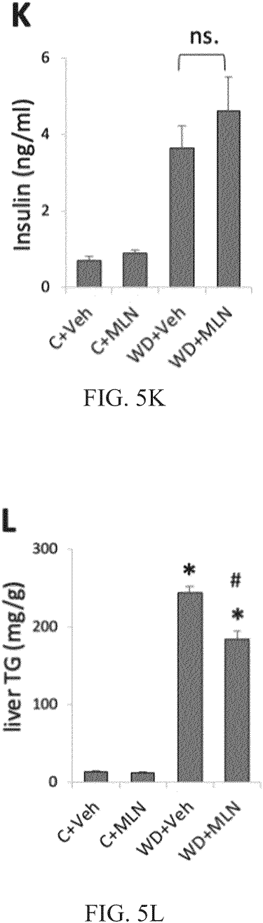

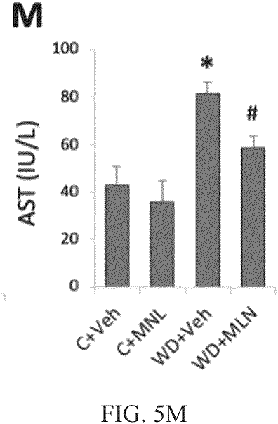

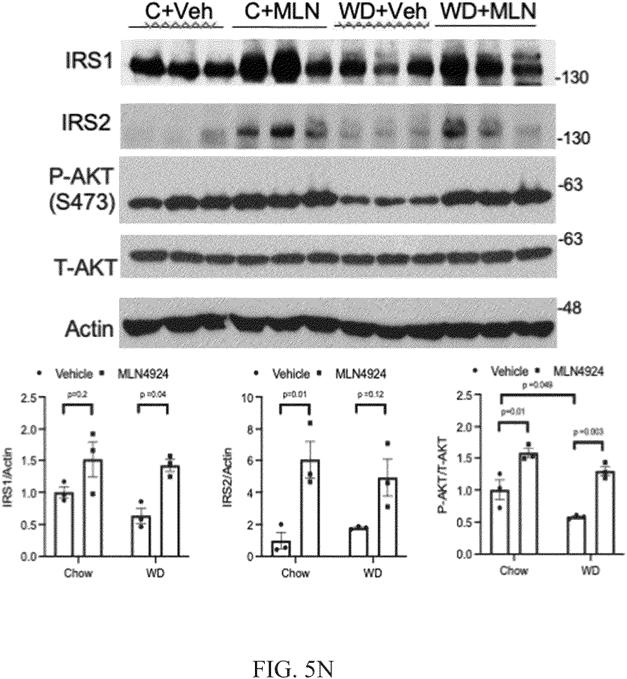

Next investigated was the glucose lowering effect of CRL inhibition in mice rendered obese and insulin resistant by 16-weeks of WD feeding. In a prevention experiment, MLN4924 treatment twice/week did not affect obesity (FIG. 5A) but completely normalized blood glucose (FIG. 5B). MLN4924 also reduced hepatic triglycerides (TG) by ~40% (FIG. 5C) and cholesterol by ~30% (FIG. 5D). After 16 weeks of MLN4924 treatment, MLN4924 did not cause transaminase (ALT) elevation in C-fed mice (FIG. 5E), suggesting that MLN4924 was well tolerated and did not cause hepatotoxicity. Furthermore, WD feeding increased serum ALT, which was significantly reduced in MLN4924-treated group (FIG. 5E), suggesting reduced liver injury by MLN4924. Next conducted was an intervention study in which MLN4924 treatment was initiated after mice were fed WD for 8 weeks that rendered mice obese and insulin resistant. MLN4924 intervention did not affect obesity (FIG. 5F), adiposity as reflected by gonadal fat (FIG. 5G), serum fatty acids (FIG. 5I) or liver glycogen (FIG. 5J), but normalized blood glucose (FIG. 5H). MLN4924 caused an increase in circulating insulin (FIG. 5K) but in this experiment the amount of the increase was not significantly greater than the level in the control (-5 ng/ml vs. ~3.5 ng/ml). MLN4924 intervention lowered hepatic steatosis as measured by liver TGs (FIG. 5L) and decreased serum AST (FIG. 5M). MLN4924 treatment increased hepatic AKT phosphorylation in chronic WD-fed mice (FIG. 5N), suggesting improved hepatic insulin sensitivity. In summary, robust evidence is provided herein that pharmacological CRL inhibition is an effective approach to lowering hyperglycemia.

A hyperinsulinemic-euglycemic clamp study was conducted in which obese mice were treated with vehicle or MLN4924. The results show MLN4924 treatment significantly increased glucose infusion rate (~30 mg/kg/min with MLN4924 vs. ~20 mg/kg/min with vehicle) indicating improved insulin sensitivity. This was associated with significantly increased plasma insulin level (~3.75 ng/ml with MLN4924 vs ~2.4 ng/ml with vehicle) during the clamp procedure. Acute MLN4924 treatment resulted in both a significant increase in plasma insulin (~0.55 with MLN4924 vs. ~0.38 ng/ml with vehicle) and a significant increase in serum c-peptide (~1.1 with MLN4924 vs. ~0.5 ng/ml with vehicle). These results confirmed that MLN4924 stimulates insulin secretion as a mechanism of action in its glucose lowering effect.

### CRL Inhibition Prolonged Insulin Action by Stabilizing IRS1 and IRS2 in Hepatocytes

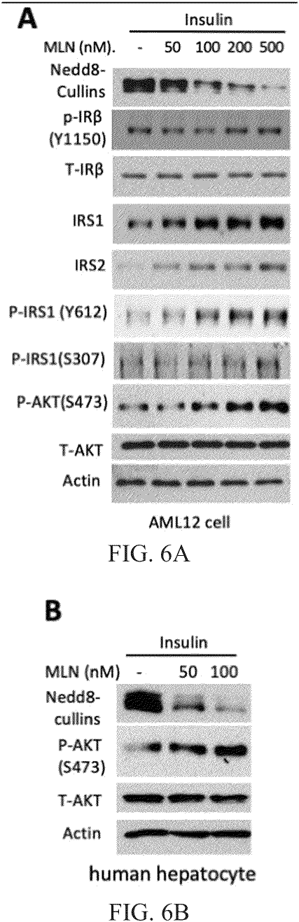

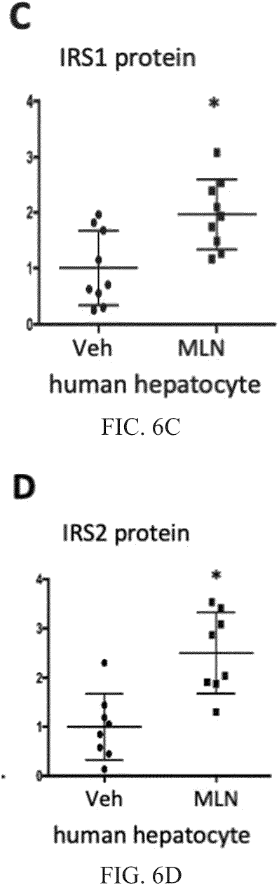

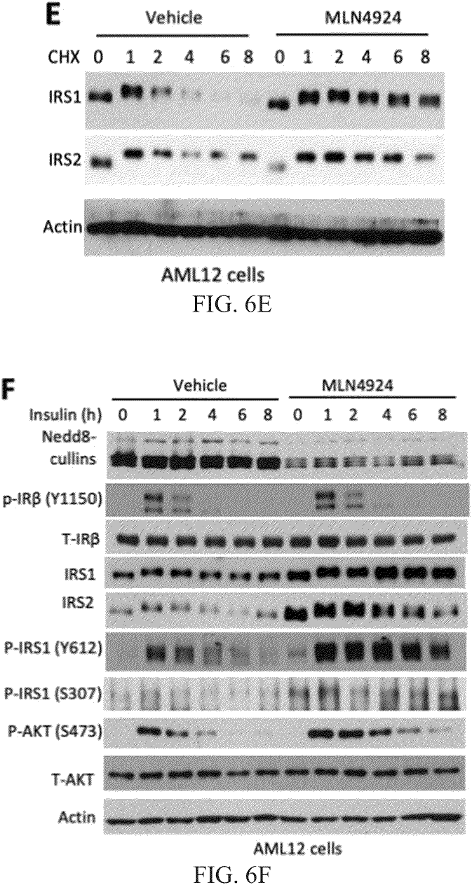

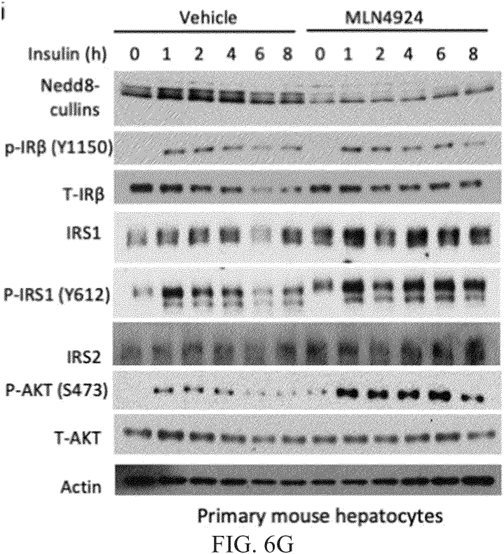

IRS proteins were highly sensitive to CRL inhibition. Modest neddylation reduction (reduced NEDD8-cullins) resulted in significantly increased IRS protein abundance and enhanced insulin action in mouse liver AML12 cells (FIG. 6A). Further, MLN4924 enhanced insulin activation of AKT (FIG. 6B) in human hepatocytes, and MLN4924 consistently increased IRS1 and IRS2 protein abundance in independent batches of human primary hepatocytes (FIGS. 6C-6D). It was further demonstrated that when AML12 cells were treated with cycloheximide (CHX) to block new IRS protein synthesis, the insulin-stimulated IRS degradation was rapid in vehicle treated control cells but was significantly delayed in MLN4924-treated cells (FIG. 6E). Finally, it was shown that inhibition of CRL by MLN4924 significantly delayed cellular insulin desensitization as evidenced by prolonged AKT phosphorylation in response to insulin in both AML12 cells and mouse hepatocytes (FIGS. 6F-6G). This effect was not associated with altered IR protein abundance or Y1150 phosphorylation of IR (a marker of IR activation by insulin), but was explained by higher IRS protein abundance and IRS1 Y612 phosphorylation, which is a marker of IRS1 activation (FIGS. 6F-6G). In summary, these findings demonstrate that CRL inhibition improves hepatocellular intrinsic insulin sensitivity by delaying IRS protein turnover. CRL inhibition did not cause persistent AKT activation in the absence of insulin but rather slows insulin signaling desensitization, which may be especially relevant during postprandial state and insulin resistant condition to enhance hepatic response to circulating insulin.

### CRL1 and CRL3 Mediates IRS Turnover in Liver Cells

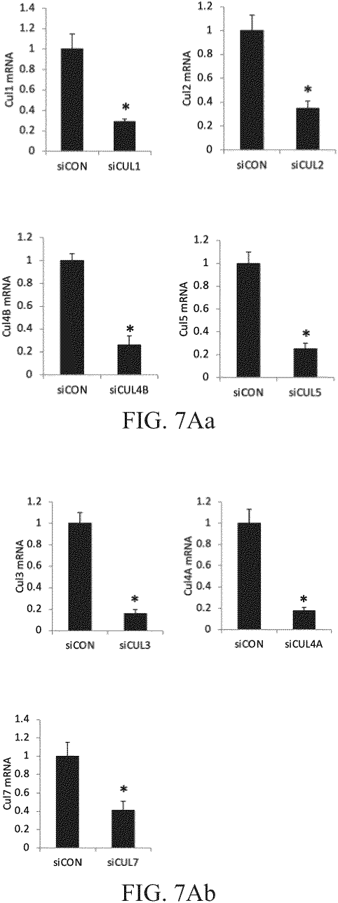

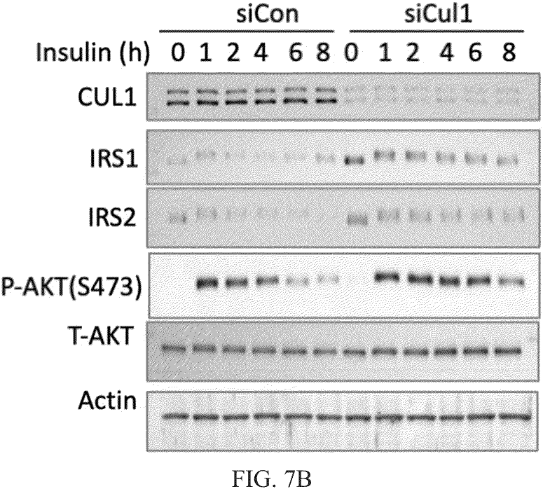

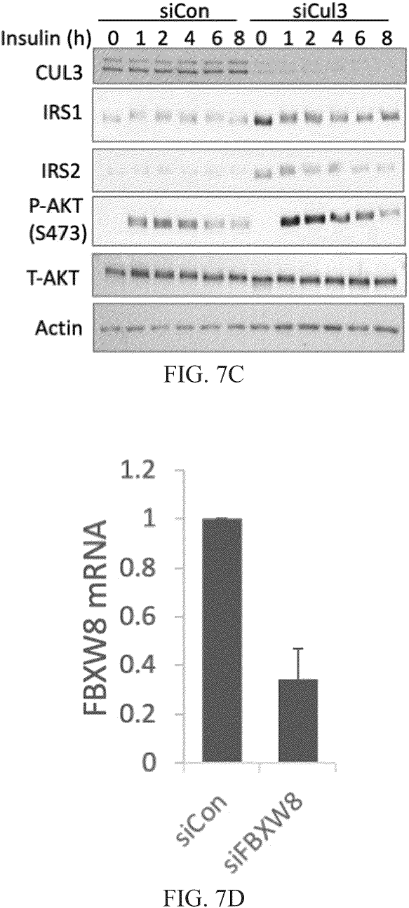

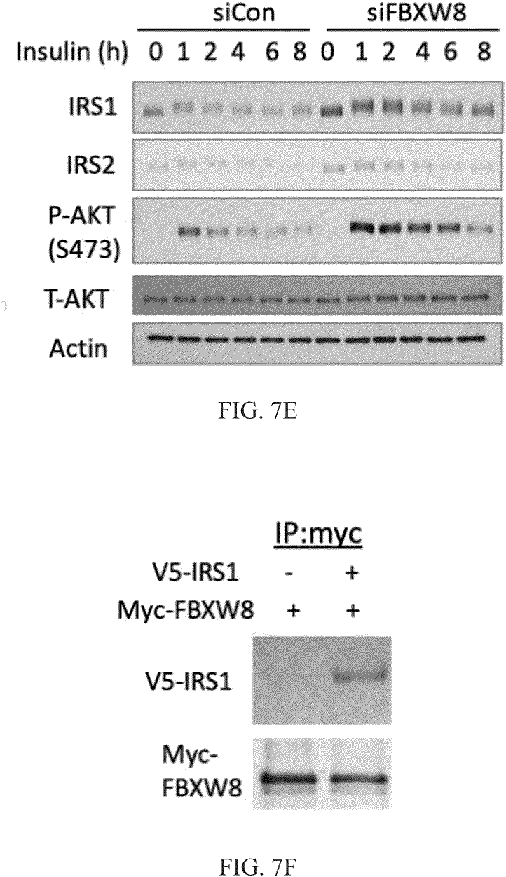

To investigate which CRL complex(s) were responsible for IRS protein degradation address this question, siRNA was used to specifically knockdown each of the 7 cullins in AML12 cells (FIG. 7A). This experiment revealed that knockdown of either Cull or Cul3 delayed IRS protein turnover and significantly enhanced insulin activation of AKT (FIGS. 7B and 7C, respectively). Furthermore, F-box/WD repeat-containing protein 8 (FBXW8), a substrate adaptor associated with CRL1, was also knocked down which increased IRS1 and IRS2 and enhanced insulin-stimulation of p-AKT (FIGS. 7D-7E). Coimmunoprecipitation (Co-IP) also detected FBXW8-IRS1 interaction (FIG. 7F). In summary, these results indicate that CRL1 and CRL3 regulate IRS protein turnover in response to insulin stimulation and are likely involved in mediating the insulin sensitizing effects of MLN4924. Furthermore, FBXW8 is a candidate IRS substrate adaptor that is known to be associated with CRL1.

### CRL Inhibition by MLN4924 Enhances Insulin Signaling and Glucose Uptake In Differentiated Muscle C2C12 Myocytes

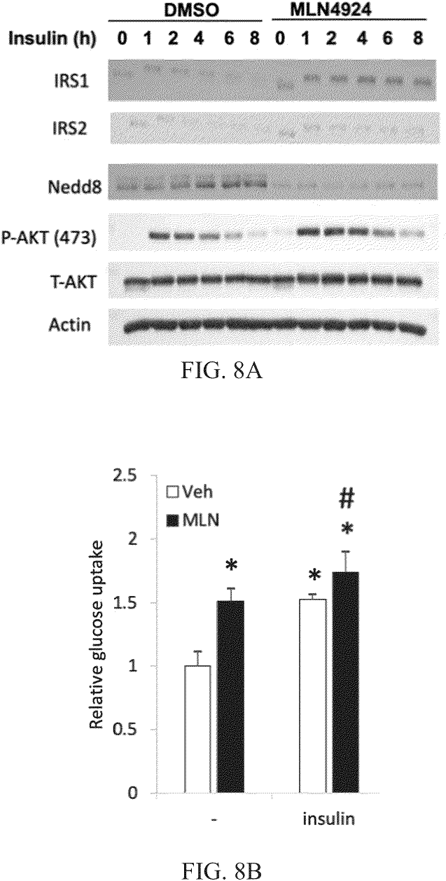

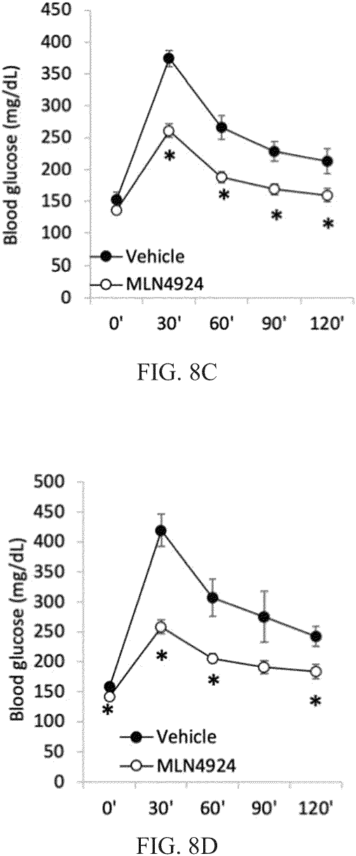

Skeletal muscle is a major organ that is responsible for the majority of the blood glucose clearance during postprandial state. Insulin resistance in muscle causes impaired insulin-stimulated glucose uptake that contributes to hyperglycemia in diabetes. To determine if CRL inhibition in muscle cells may contribute to the insulin sensitizing and glucose lowering effect in obese mice, differentiated C2C12 myocytes were treated with MLN4924 to inhibit CRLs (FIG. 8A). Upon insulin stimulation, MLN4924 treated cells showed delayed IRS1 and IRS2 degradation, which correlated with increased insulin activation of AKT phosphorylation (FIG. 8A). Consistently, enhanced insulin signaling resulted in increased glucose uptake in C2C12 cells (FIG. 8B). To further determine if MLN4924 treatment increases muscle glucose clearance, a glucose tolerance test (GTT) was performed, in which reduction of glucose excursion is mainly attributed to blood glucose clearance by muscle. Indeed, it was found that in both C-fed and 3-week WD-fed mice, MLN4924 treatment significantly improved glucose tolerance in GTT (FIGS. 8C-8D). In summary, these results indicate that improved muscle insulin sensitivity and glucose uptake is another mechanism by which MLN4924 lowers blood glucose.

### CRL Inhibition by TAS4464 Reduced Plasma Glucose in Obese Mice

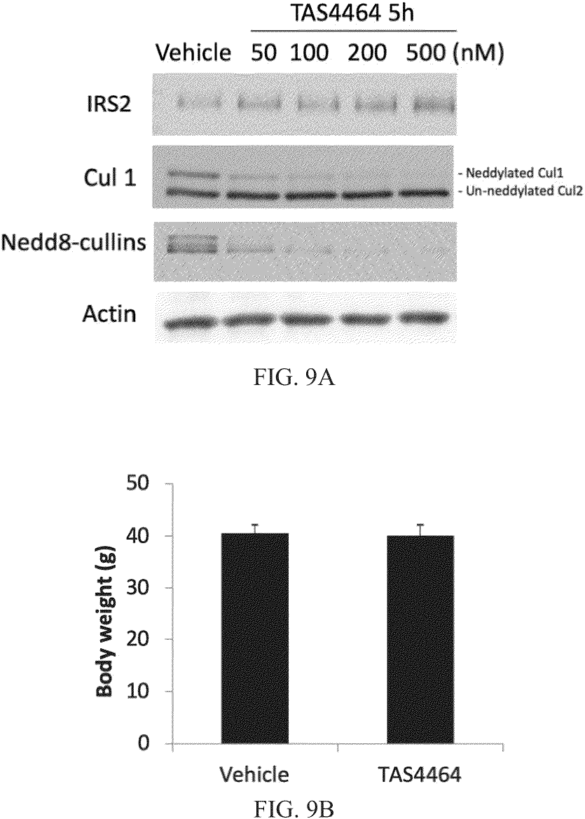

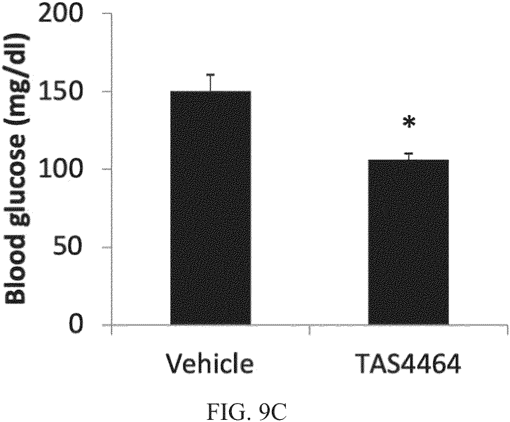

To further investigate determine the robustness of CRL inhibition on glucose lowering, tests were conducted using the potent NAE1 inhibitor TAS4464 (7H-Pyrrolo[2,3-d]pyrimidin-4-amine, 7-[5-[(aminosulfonyl)amino]-5-deoxy-beta-D-ribofuranosyl]-5-[2-(2-ethoxy-6-fluorophenyl) ethynyl]-. TAS4464 treatment significantly inhibited total neddylated cullins and neddylated cullin 1, which resulted in increased IRS protein in AML12 cells in vitro (FIG. 9A). Next, we fed mice with WD for 8 weeks to first render them obese and insulin resistant (FIG. 9B). We then treated the experimental mice with TAS4464 twice (5 pm on day 1 and 9 am on day 2) and measured blood glucose after 6-hour fast. The obese control mice treated only with vehicle showed hyperglycemia of ~150 mg/dl (FIG. 9C). Treatment of obese mice with TASS4464 reduced the blood glucose to ~100 mg/dl. These results provide robust additional evidence that CRL inhibition results in plasma glucose reduction in obese and insulin resistant condition.

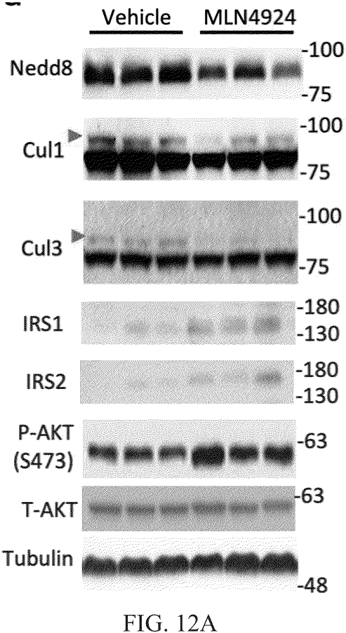

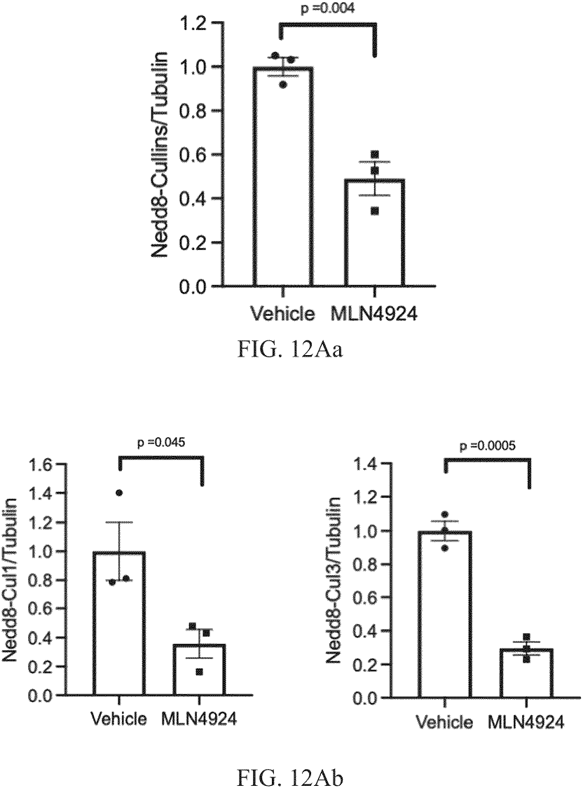

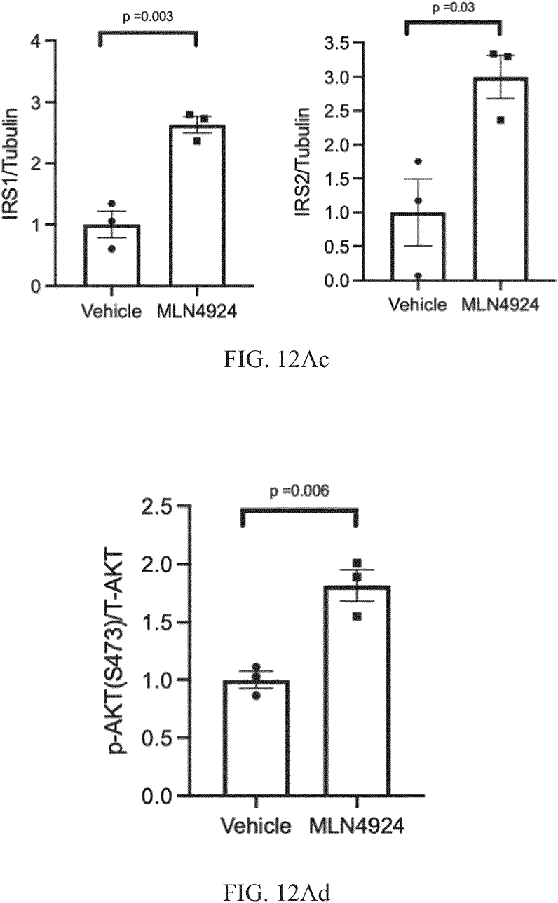

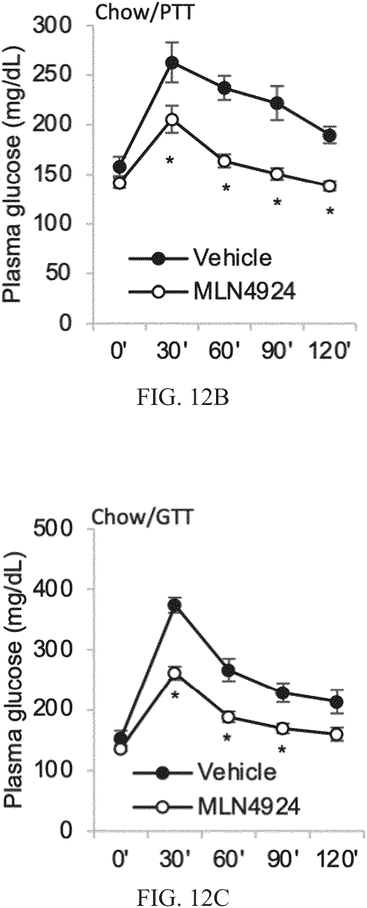

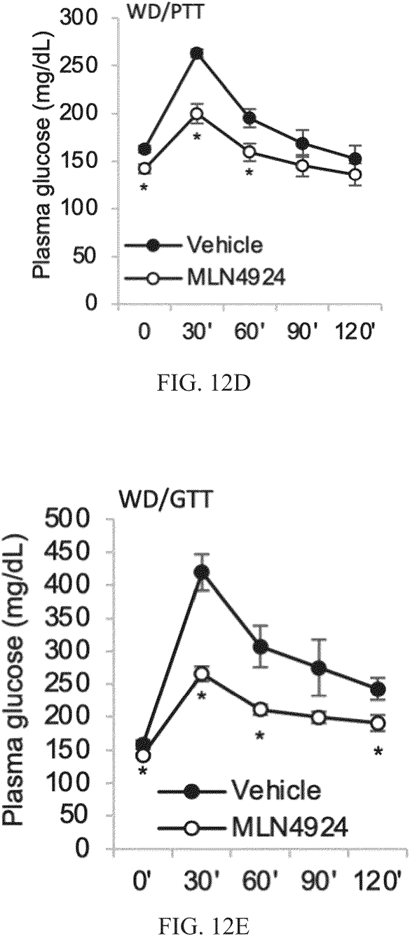

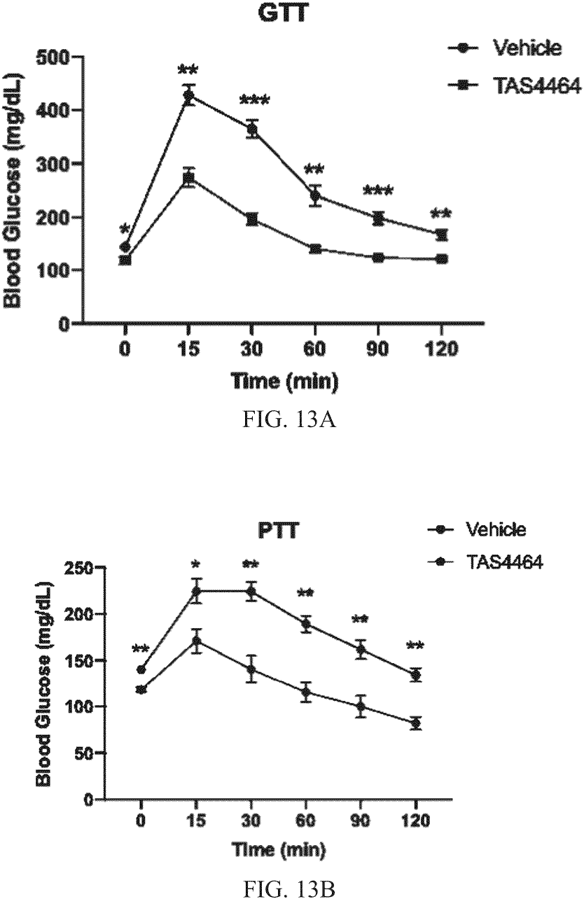

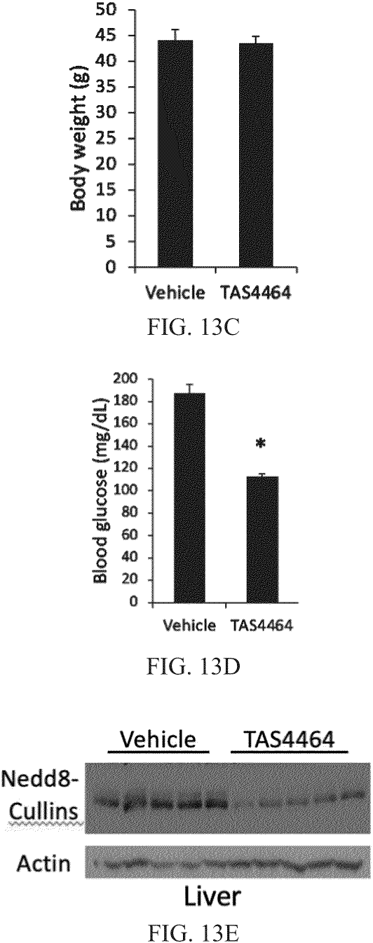

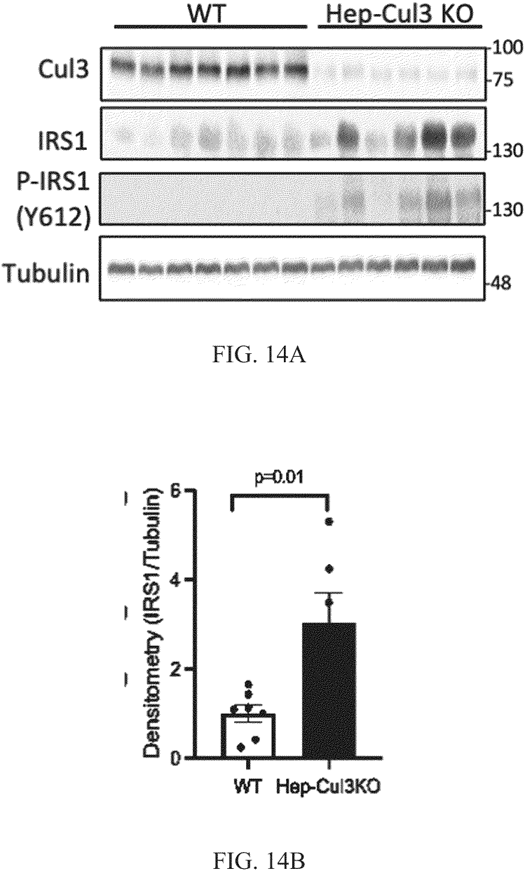

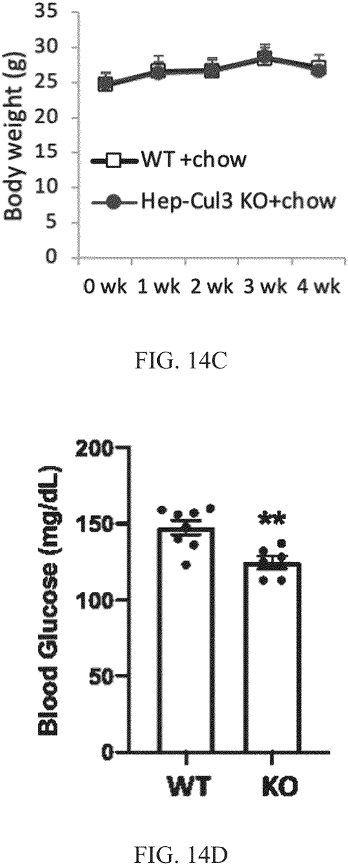

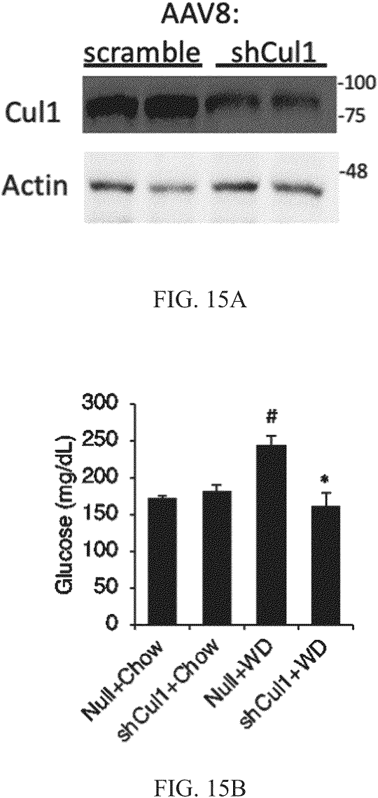

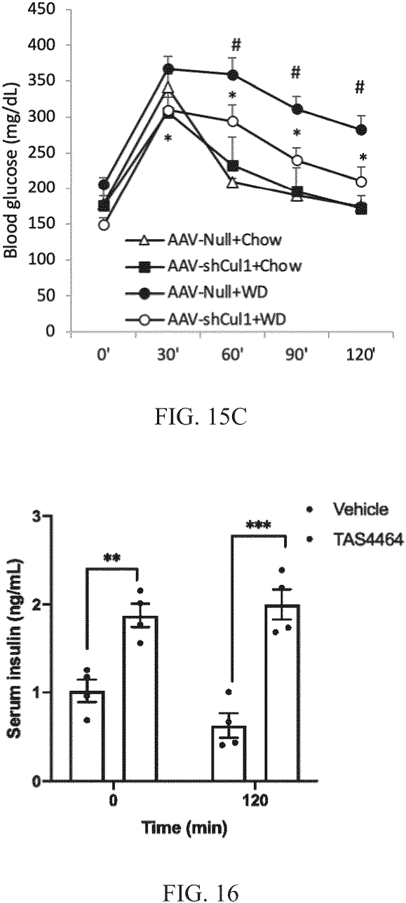

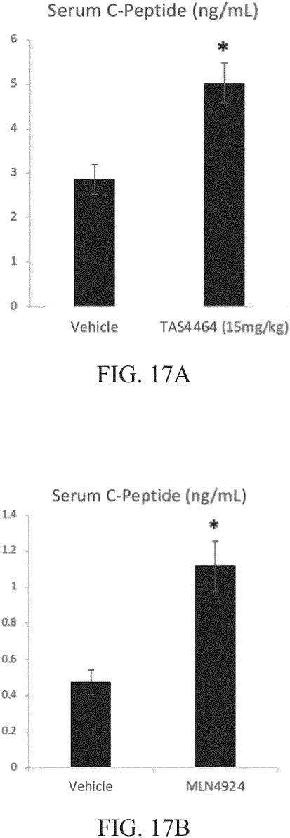

Nedd8 conjugation to the cullin portion of a CRL is required for efficient activity of the CRL. Therefore, when the conjugation of Nedd8 to the cullin is inhibited, e.g., by MLN4924 or TAS4464, the CRL is inactivated and degradation of the CRL-targeted proteins (IRS proteins) is mitigated. Results disclosed herein demonstrate that inhibition of CRL neddylation by MLN4924 and TAS4464 reduced blood glucose in obese mice and that MLN4924 completely normalized blood glucose without affecting obesity or circulating insulin in obese and insulin resistant mice. Without wishing to be bound by theory, this beneficial effect can be attributed to the following mechanisms: (1) inhibition of CRLs delays IRS1 and IRS2 protein turnover and thus prolongs insulin action in hepatocytes and myocytes, (2) enhanced hepatic insulin signaling reduced hepatic glucose production in mice, and (3) in muscle cells, enhanced insulin signaling increased glucose uptake. The combined insulin sensitizing effect of CRL inhibition in liver and muscle cells contribute to reduced blood glucose. The results demonstrate that insulin signaling is subjected to control by CRLs through the modulation of IRS protein stability, which supports the finding herein that pharmacological inhibition of CRLs is effective in improving insulin sensitivity and lowering blood glucose and thus can be used as a therapy to treat hyperglycemia and insulin resistance, for example in type-2 diabetes patients. Chronic MLN4924 treatment was well tolerated in mice. Notably, this therapeutic strategy is mechanistically distinct from all current existing classes of antidiabetic drugs including biguanides, sulfonylureas, meglitinides, thiazolinediones, SGLT2 inhibitors, incretin mimetics, DPP IV inhibitors, and α-glucosidase inhibitors, and thus provides the opportunity for single-drug or combinational therapies for optimal glycemia, given that glycemia is still poorly controlled in many obese and diabetic patients by therapeutics conventionally used to treat hyperglycemia. Further results are shown in FIGS. 12A-17B.

Therefore, provided above are in vivo and in vitro data which demonstrate that pharmacological inhibition of CRL is effective in improving insulin sensitivity and glycemia in type-2 diabetes. Inhibition of CRL neddylation by MLN4924 delays IRS protein turnover and enhances insulin signaling as evidenced by increased AKT (Protein kinase B) phosphorylation in liver and muscle cells. Further, it is shown that specific knockdown of Cul1 and Cul3 in liver AML12 cells stabilizes IRS protein and enhances insulin signaling, which recapitulates the insulin sensitizing effect of MLN4924. These data support that the insulin sensitizing effect of MLN4924 is a result of inhibition of Cul1 and Cul3 by MLN4924. In mice, it is shown through PTT and GTT that MLN4924 significantly decreases blood glucose excursion in chow-fed and in WD-fed mice, indicating decreased hepatic glucose production and improved muscle glucose clearance. The effect of MLN4924 in promoting muscle glucose uptake is also demonstrated in cultured C2C12 cells in vitro. The insulin sensitizing effect of MLN4924 in liver and muscle accounts for the completely normalized blood glucose in 16-week WD-fed mice. In addition, evidence is provided herein that CRL inhibition by TAS4464 also achieved significant glucose lowering effect in obese mice, further supporting the conclusion that lowering blood glucose by inhibiting CRL is effective and robust. These data demonstrate that inhibition of NAE1 or inhibition of CRL activity by other modes is an effective therapeutic approach in lowering blood glucose as an anti-diabetes treatment.

### Experimental II

### Methods

### Reagents

MLN4924 was purchased from MedChemExpress, Inc. (Monmouth Junction, NJ). Insulin (Novolin, NDC 0169-1833-11) was purchased from Novo Nordisk Inc. (Plainsboro Township, NJ). cycloheximide, wortmannin, Torin1, rapamycin and antibodies against Nedd8 (#2754, Lot. 2), Cul3 (#2759, Lot. 2), Cul4A (#2699, Lot. 2), NAE1 (#14321, Lot. 1), p-IRβ (Y1150)(#3918, Lot.2), T-IRβ (#3025, Lot. 10), IRS1 (#2382, Lot. 10), IRS2 (#4502, Lot. 5), p-AKT(S473)(#4060, Lot. 24), p-AKT(T308) (#4056, Lot. 19), T-AKT (#4691, Lot.20), p-S6(S240/244) (#2215, Lot.14), T-S6 (#2217, Lot.5), p-4E-BP (T37/46) (#2855, Lot.20), Myc tag (#2278S, lot.7), V5 tag (#13202S, Lot.6), T-4E-BP (#9452, Lot.10), Histone 3 (#9715S, were purchased from Cell Signaling Technology Inc. (Danvers, MA). Antibodies against p-IRS1 (Y612) (#44-816G, Lot.SG255272), p-IRS1(S307), (#PAI-1054, Lot.TG265968), Cul1 (#32-2400, Lot.UJ297298), and Cul2 (#51-1800, Lot.UA281866) were purchased from Invitrogen (Carlsbad, CA). Antibodies against Cul4B (#12916-I-AP) is purchased from Proteintech, Inc. (Rosemont, IL). Antibodies against Cul7 (#C1743, Lot.127M4759V) and 2-hydroxypropyl-b-cyclodextrin (#332607) were purchased from Sigma Aldrich (St. Louis, MO). Antibodies against Actin (Ab3280) was purchased from Abcam (Cambridge, MA). Insulin ELISA kit (EZRMI-13K) was purchased from Millipore (Burlington, MA). Aspartate aminotransferase (ALT) assay kit and triglyceride assay kit were purchased from Pointe Scientific (Canton. MI). Fatty acid assay kit (K612) was purchased from BioVision, Inc. (Milpitas, CA). MBL anti-V5-tag magnetic beads, Lipofectamine RNAiMAX reagent and Lipofectamine 3000 reagent were purchased from ThermoFisher Scientific (Waltham, MA).

### Cell Culture and Transfection

AML12 cells were a generous gift from Dr. Yanqiao Zhang (Northeast Ohio Medical University). AML12 cells were cultured in DMEM medium supplemented with a mixture of insulin-transferrin-selenium (#41400-045, ThermoFisher, Waltham, MA). For experiments, cells were cultured in serum free medium overnight before various treatments were initiated. The siGENOME SMARTpool siRNA and siControl were purchased from Dharmacon, Inc. (Lafayette, CO). The siRNA was transfected with Lipofectamine RNAiMAX reagent in a final concentration of 25 nM recommended by the manufacturer. Primary human hepatocytes and primary mouse hepatocytes were obtained from the Cell Isolation Core at the University of Kansas Medical Center. Treatments were initiated on the same day of isolation and completed within 24 hours.

INS-1 832/13 rat insulinoma cell line was purchased from Sigma-Aldrich (St. Louis, MO). Cells were cultured in 24-well plate in RPMI-1640 growth medium until 100% confluent. Cells were then treated with vehicle (DMSO) or 500 nM MLN4924 for 5 hrs. Cells were then incubated with 0.5 ml secretion assay buffer (SAB, 114 mM NaCl, 4.7 mM KCl, 1.2 mM KH₂PO₄, 1.16 mM MgSO₄, 20 mM HEPES pH 7.2, 25.5 mM NaHCO₃, 2.5 mM CaCl₂, and 0.2% BSA) containing 2.5 mM glucose and either vehicle or 500 nM MLN4924 for 1 hr. This step was repeated once. Next, SAB containing 12 mM glucose (0.5 ml) was added to cells to stimulate insulin secretion for 1 hr. The SAB was then collected and cells were lysed in 0.2 ml 1X RIPA buffer. Insulin concentration in SAB and cell lysates was measured with an ELISA assay kit.

### Co-immunoprecipitation.

The pcDNA3-myc3-CUL1 (Addgene plasmid # 19896) and pcDNA3-myc-CUL3 (Addgene plasmid # 19893) were a gift from Yue Xiong (University of North Carolina at Chapel Hill). The pcDNA3.1-V5-IRS1 plasmid was a gift from Zhen-Qiang Pan (The Icahn School of Medicine at Mount Sinai, New York). Cells were transfected with expression plasmids as indicated with Lipofectamine 3000 following the manufacturer’s instruction. After 24 h, cells were lysed in co-immunoprecipitation buffer (20 mM Tris-Cl, pH=7.5, 120 mM NaCl, 1 mM EGTA, 1 mM ETDA, 1% NP-40) supplemented with protease and phosphatase inhibitor cocktail on ice for 1 h. Cell lysates were centrifuged at 10,000xg for 10 minutes at 4° C. Supernatant was transferred to a new tube and anti-V5 magnetic beads were added. After overnight incubation with rotation at 4° C., magnetic beads were washed in PBS-T (1X PBS + 0.1% tween-20) 3 times. Immunoprecipitated proteins were eluted by incubating the magnetic beads in laemmli sample buffer at 80° C. for 5 minutes. Eluted protein lysates were used for SDS-PAGE and Western blotting.

### Animal Experiments

WT male C57BL/6J mice were purchased from the Jackson Lab (Bar Harbor, ME). Western diet (TD. 88137, Envigo Inc. Indianapolis, IN) contains 42% fat calories and 0.2% cholesterol. Mice were housed in micro-isolator cages with corn cob bedding under 7 am - 7 pm light cycle and 7 pm -7 am dark cycle. MLN4924 was prepared in 10% 2-hydroxypropyl-b-cyclodextrin to a final concentration of 3 mg/ml. All mice were fasted for 6 h from 9 am to 3 pm before euthanasia. All animals received humane care according to the criteria outlined in the “Guide for the Care and Use of Laboratory Animals.” All animal protocols were approved by the Institutional Animal Care and Use Committee at the University of Kansas Medical Center and the University of Oklahoma Health Sciences Center.

### Lipid Measurements

Lipids were extracted from liver tissues with a mixture of chloroform: methanol (2:1; v: v), dried under nitrogen, and resuspended in isopropanol containing 1% triton X-100. Liver triglyceride was measured by a colorimetric assay kit following the manufacturer’s instruction. Plasma fatty acid concentration was measured with a colorimetric assay kit following the manufacturer’s instruction.

### Pyruvate Tolerance Test (PTT), Glucose Tolerance Test (GTT) and Insulin Tolerance test (ITT)

Mice were treated with 2 doses of MLN4924 as described. After the second injection at 9 am, mice were fasted for 6 h from 9 am to 3 pm. Mice were injected intraperitoneally 2 mg/kg glucose for GTT, 2 mg/kg sodium pyruvate for PTT, and 0.5 U/kg insulin for ITT. Blood glucose was measured with a OneTouch Ultra glucose meter.

### Tissue Lysate Preparation and Western Blotting.

Human normal livers (less than 5% steatosis) and histologically confirmed NASH livers were obtained from either the University of Kansas Liver Center or the Liver Tissue Cell Distribution System (LTCDS) at the University of Minnesota. Human and mouse tissue samples were homogenized in 1XRIPA buffer containing 1% SDS and protease inhibitors. After incubation on ice for 1 hour, the samples were centrifugation and supernatant was transferred to a new tube. The protein lysate was mixed with equal volume of 2X laemmli buffer and incubated at 95° C. for 5 minutes and used for SDS-PAGE and immunoblotting. ImageJ software (NIH) was used to determine band intensity.

### Real-Time PCR

Total liver RNA was purified with Trizol (Sigma-Aldrich, St. Louis, MO). Total liver RNA was used in reverse transcription with Oligo dT primer and SuperScript III reverse transcriptase (Thermo Fisher Scientific, Grand Island, NY). Real-time PCR was performed on a Bio-Rad CFX384 Real-time PCR system with iQ SYBR Green Supermix (Bio-rad, Hercules, CA). The comparative CT (Ct) method was used to calculate the relative mRNA expression. The relative mRNA expression was expressed as 2 ^{-ΔΔCt} with the control group arbitrarily set as “1”.

### Statistics.

Unpaired Student’s t-test was used for 2 group comparison. For multigroup comparison, one-way or two-way ANOVA and Tukey post hoc test were used. A p < 0.05 was considered statistically significant.

### Results and Discussion

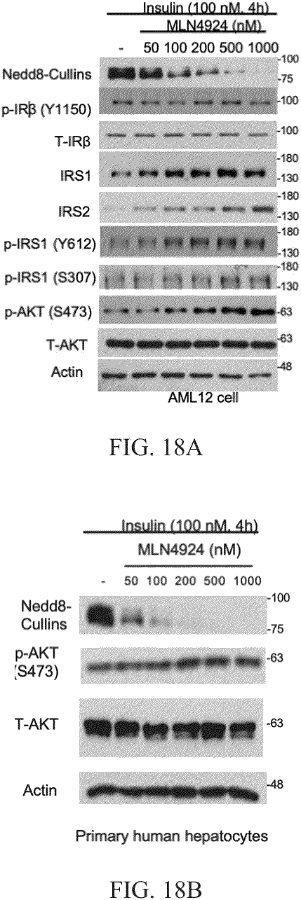

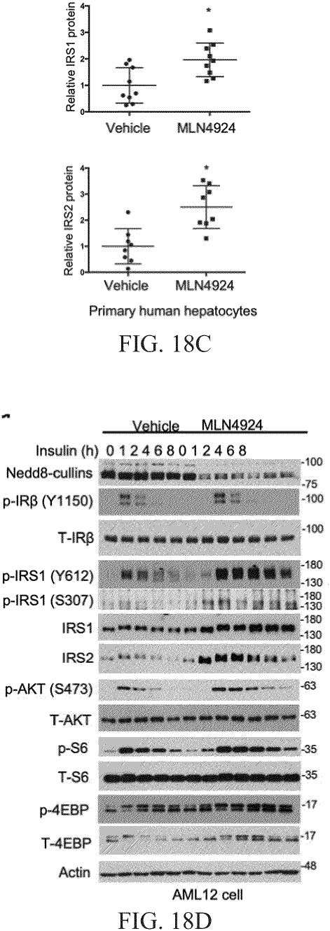

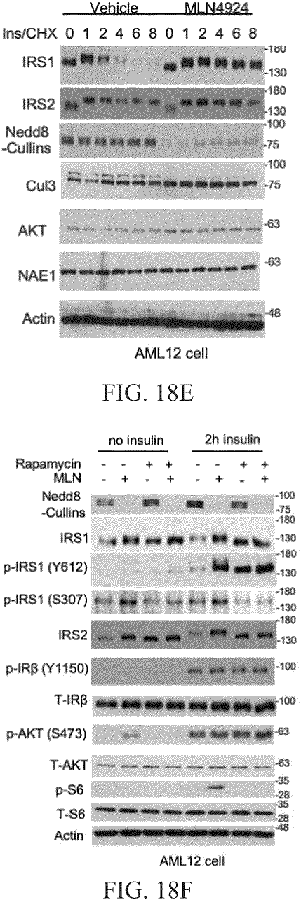

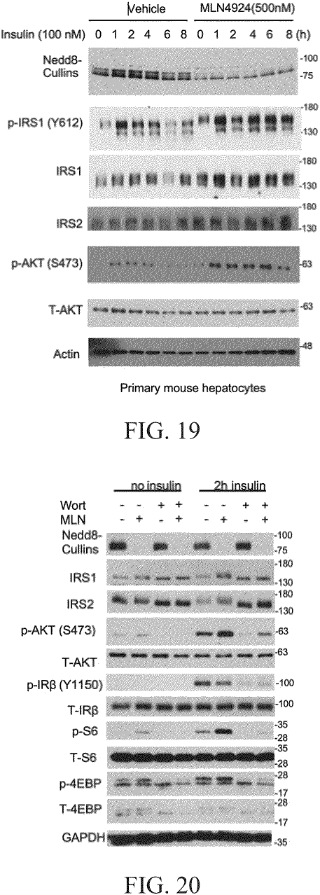

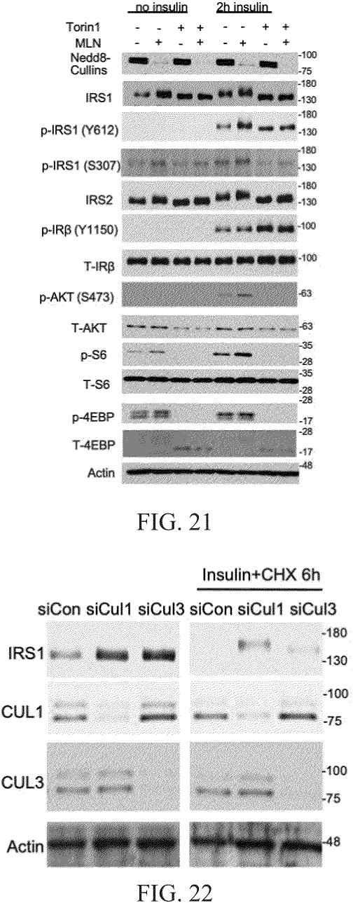

The present work demonstrates that neddylation inhibition prolongs insulin action by stabilizing IRS protein in hepatocytes. To determine the effect of neddylation inhibition on hepatic insulin signaling, we first measured the key components of the insulin signaling cascade in MLN4924-treated mouse liver AML12 cells and primary human and mouse hepatocytes. Under insulin stimulated condition, MLN4924 dose-dependently increased total IRS1 and IRS2 protein, but not total insulin receptor (IR) or AKT (FIG. 18A). This resulted in increased AKT S473 phosphorylation that correlated with enhanced IRS1(Y612) phosphorylation but not IR (Y1150) phosphorylation (FIG. 18A). MLN4924-mediated insulin sensitization is highly dose-dependent and a partial neddylation inhibition was sufficient to enhance hepatic insulin sensitivity (FIG. 18A). Similarly, MLN4924 treatment increased IRS protein abundance and insulin-stimulated AKT activation in primary human hepatocytes (FIGS. 18B-C) and primary mouse hepatocytes (FIG. 19), which ruled out a cell type-specific effect. When cells were stimulated with insulin in time course, the presence of MLN4924 significantly delayed insulin signaling desensitization at the IRS1(Y612) and AKT(S473) levels in AML12 cells and primary mouse hepatocytes (FIGS. 18D, 19). This effect was not associated with IR (Y1150) phosphorylation but was explained by higher IRS protein abundance. Activation of mTORC1 downstream of insulin signaling mediates IRS1 serine phosphorylation and subsequent ubiquitin-proteasomal degradation. In the presence of MLN4924, IRS1 S307 phosphorylation was also higher (FIG. 18D), suggesting that delayed IRS1 inactivation was due to delayed IRS1 protein degradation but not reduced IRS1 serine phosphorylation. When cycloheximide (CHX) was used to block protein synthesis, insulin-induced IRS degradation appeared to be rapid in vehicle-treated cells and was significantly delayed by MLN4924 (FIG. 18E). In contrast, other proteins including AKT, Cul3 and NAE1 appeared to be more stable (FIG. 18E). Under insulin-stimulated condition, blocking the mTORC1-mediated IRS feedback inhibition by rapamycin decreased IRS1 serine phosphorylation (lower IRS1 S307 phosphorylation and lack of insulin-induced IRS band shift) and increased IRS protein to similar levels in MLN4924-treated cells although insulin-induced IRS1 S307 phosphorylation and band shift were preserved under MLN4924 treatment (FIG. 18F). MLN4924 did not further increase IRS protein in the presence of rapamycin (FIG. 18F), supporting that mTORC1-mediated serine/threonine phosphorylation of IRS and CRL-mediated degradation are two sequential steps in the same insulin desensitization mechanism. These rapamycin effects on IRS proteins could be re-produced by using a PI3K inhibitor wortmannin and a TORC½ inhibitor Torin 1, both of which blocked downstream mTOR activation, except that both inhibitors also decreased downstream AKT activation owing to the known roles of PI3K and TORC2 in mediating AKT phosphorylation (FIGS. 20-21). In summary, these findings indicate that CRL inhibition improves hepatocellular intrinsic insulin sensitivity by delaying IRS protein turnover. CRL inhibition did not cause persistent AKT activation in the absence of insulin but rather slowed insulin signaling desensitization (FIG. 18D, which may be especially relevant under hepatic insulin resistant condition to enhance hepatic responsiveness to higher circulating insulin.

### Cullin 1 and Cullin 3 Mediate IRS Protein Degradation in Hepatocytes

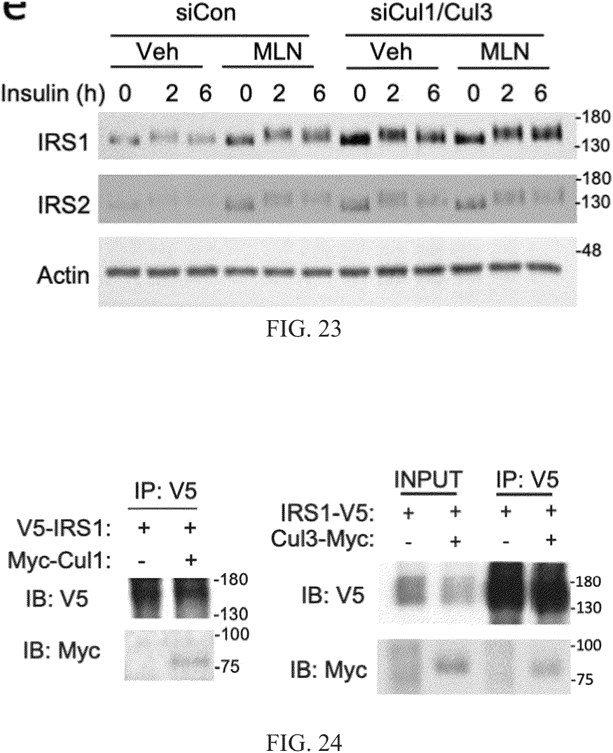

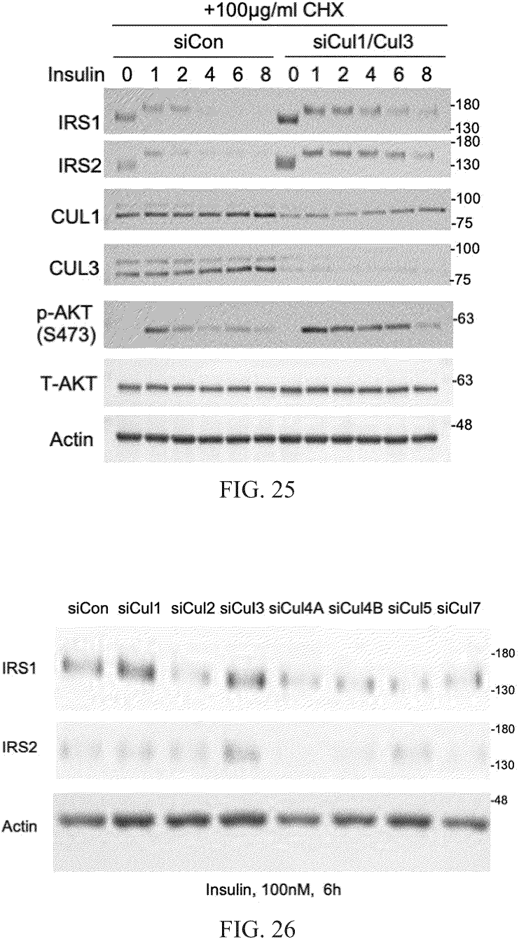

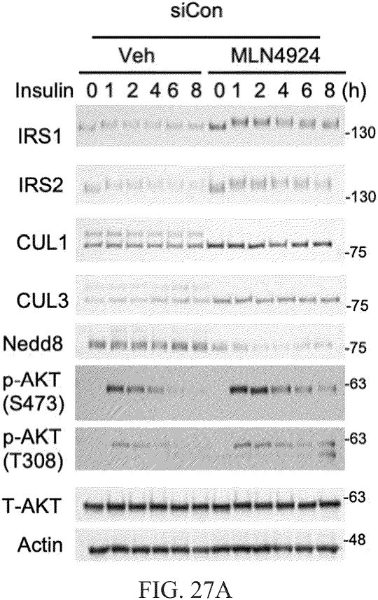

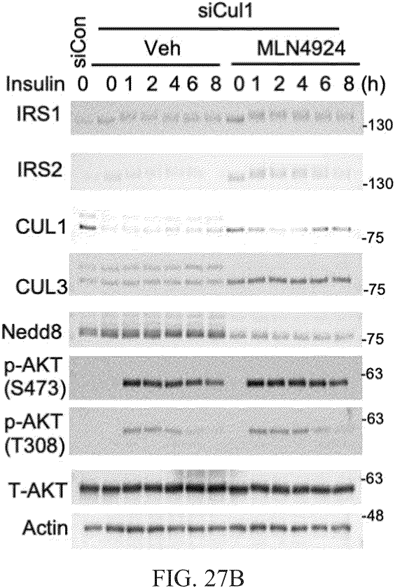

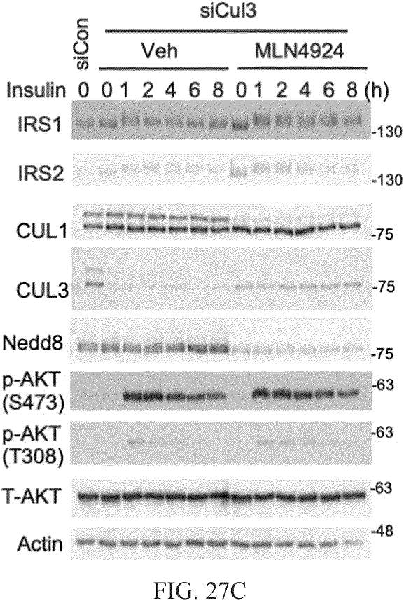

To identify the cullin members that are involved in IRS regulation, we performed a screening experiment by specifically knocking down each of the 6 mammalian canonical cullins (Cul1, Cul2, Cul3, Cul4A, Cul4B, Cul5) and the atypical Cul7 in AML12 cells (FIG. 7Aa-7Ab). Efficient knockdown of each cullin at the protein level was confirmed by Western blotting (not shown). Under insulin stimulated condition that induces rapid IRS protein degradation within 6 hr (FIG. 18E), knockdown of Cul1 or Cul3 appeared to delay IRS turnover (FIG. 22, FIG. 26). Knockdown of other cullins, including Cul7 which was previously reported to regulate IRS1 in mouse embryonic fibroblasts, did not affect IRS stability in liver cells (FIG. 26), indicating that a CRL complex exhibits cell-type specific functions. Knockdown of either Cul1 or Cul3 increased cellular response to insulin-stimulated AKT activation and thus recapitulated the effect of MLN4924 (FIGS. 7B-7C), suggesting that the loss of either Cul1 or Cul3 was not fully compensated for by the other cullin. In Cul1 or Cul3 knockdown cells, further MLN4924-mediated IRS enrichment and insulin sensitization appeared to be limited but not fully abolished (FIGS. 27A-C), likely due to neddylation inhibition on the remaining Cul1 and Cul3. However, simultaneous knockdown of Cul1 and Cul3 largely abolished further IRS protein stabilization caused by MLN4924 (FIG. 23), indicating that Cul1 and Cul3 is responsible for most, if not all, of the CRL-mediated IRS protein degradation in liver cells. In further support, Cul1 and Cul3 can be co-immunoprecipitated with IRS1 protein (FIG. 24), and simultaneous knockdown significantly delayed insulin-stimulated IRS protein degradation when protein synthesis was blocked by CHX (FIG. 25).

Acute CRL inhibition enhances hepatic insulin signaling and reduces hepatic glucose production.

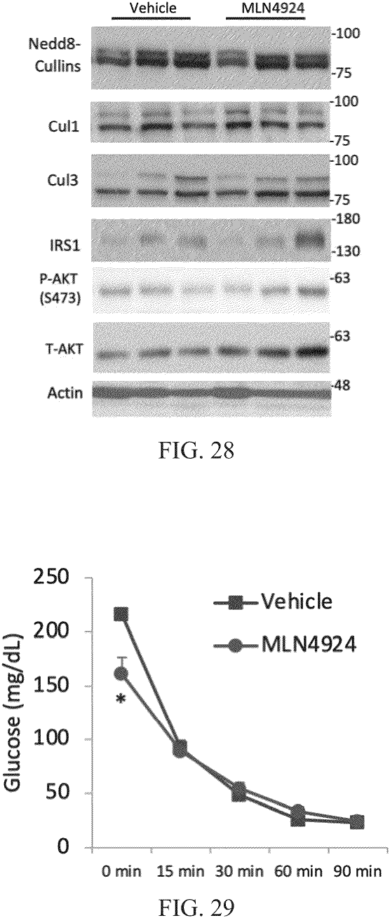

To further explore the hepatic insulin sensitizing effect of neddylation inhibition in vivo, we administered MLN4924 to chow-fed mice twice via subcutaneous (SQ) injection (5 pm on day 1 and 9 am on day 2) and studied the acute effect of MLN4924. The dose of 60 mg/kg is in line with published literature. MLN6924 treatment effectively reduced hepatic cullin neddylation (FIG. 12A, 12Aa-12Ad). Consistent with our in vitro findings, MLN4924 increased hepatic IRS proteins and AKT phosphorylation (FIG. 12A-12Ad). Under chow condition, MLN4924 did not significantly decrease basal glucose levels after 6 h fasting, but pyruvate tolerance test (PTT) and glucose tolerance test (GTT) revealed significantly reduced hepatic glucose production and improved overall glucose tolerance (FIGS. 12B-C). Similar improvement was produced by acute MLN4924 administration in mice after a brief 4-week Western diet (WD) feeding (FIGS. 12D-E). Notably, the peak blood glucose at 30 minutes post pyruvate and glucose challenge was significantly lower in mice injected with MLN4924, which consistently supports hepatic action of MLN4924 in reducing glucose production. In addition to liver, skeletal muscle is another organ that quantitatively contribute to plasma glucose homeostasis. However, MLN4924 did not inhibit cullin neddylation or increase IRS protein abundance or AKT phosphorylation in skeletal muscle (FIG. 28). Furthermore, insulin tolerance test showed that, despite lower basal glucose in the MLN4924-treated group, both groups showed similarly decreased plasma glucose to ~25 mg/dL in response to insulin after 1 h (FIG. 29). These results suggest that MLN4924 may have very limited distribution and effects in skeletal muscle to contribute to overall glucose lowering in mice.

MLN4924 intervention normalized blood glucose independent of obesity in 16-week WD-fed mice.

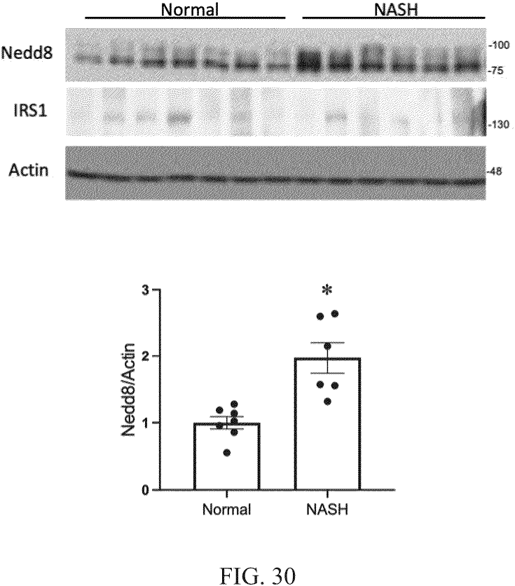

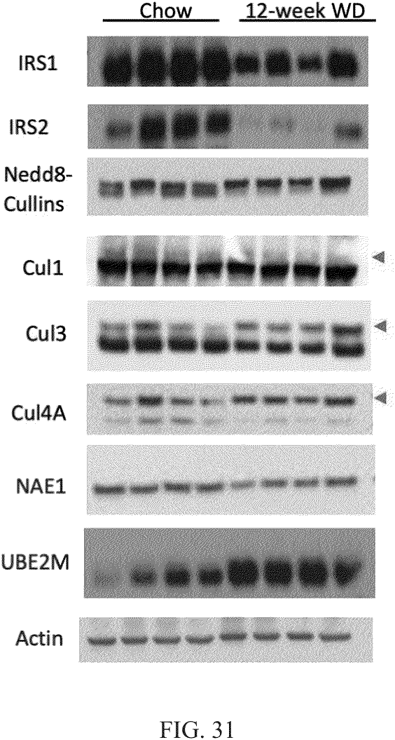

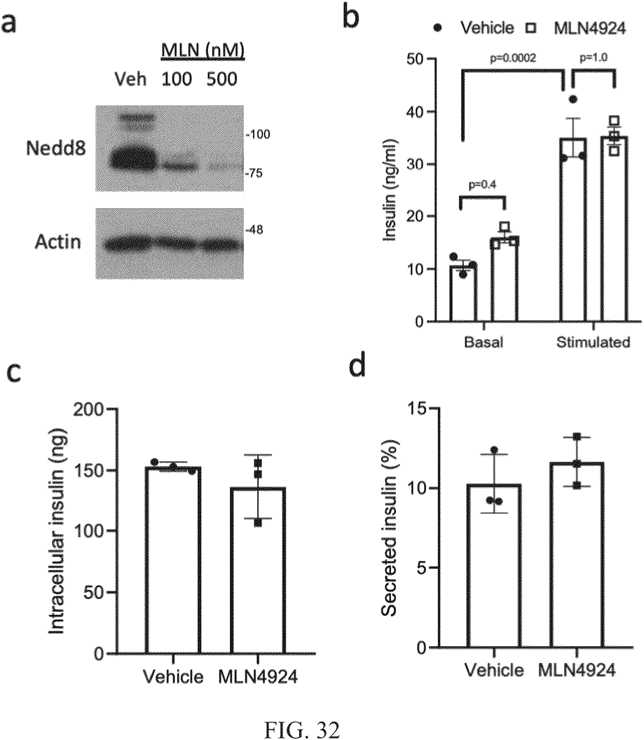

It is known that impaired IRS function is a major underlying cause of hepatic insulin resistance in fatty liver. Interestingly, we found that human fatty livers showed increased cullin neddylation (FIG. 30F). However, IRS protein was barely detectable in most human liver samples and showed large variation (FIG. 30), possibly due to the rapid turnover rate of IRS protein and the limitation of obtaining human liver samples collected under a uniformly controlled condition. We therefore turned to WD-induced murine fatty livers and found strongly decreased hepatic IRS protein abundance (FIG. 31). Hepatic neddylated Cul3 was increased while neddylated Cul1 appeared to be unaltered in murine fatty livers (FIG. 31). Cul4A neddylation was also increased in murine fatty livers (FIG. 31). Increased hepatic cullin neddylation in fatty liver did not correlate with the Nedd8 E1 enzyme NAE1, but correlated with increased UBE2M (UBC12) (FIG. 31), the major mammalian Nedd8 E2 enzyme that was recently reported to be induced under various cellular stress conditions, providing a possible explanation of hyper-neddylation of some cullins in fatty liver. These findings led us to further investigate the potential of targeting CRLs for hepatic insulin sensitization in a pathologically relevant fatty liver setting. However, a recent study has reported that neddylation inhibition suppresses adipogenesis and MLN4924 administration via i.p. injection significantly reduces obesity in high fat diet-fed mice (19), which presents a major limitation in defining obesity-independent insulin sensitizing effect of chronic MLN4924 treatment. To circumvent this technical limitation, we validated a dosing protocol by employing SQ administration of MLN4924 (60 mg/kg, once every 2 days), which did not affect diet-induced obesity (FIG. 5A). This was possibly because SQ MLN4924 administration minimized direct visceral fat exposure to MLN4924 compared to i.p. injection. By using this dosing protocol, we found that MLN4924 intervention for 9 weeks completely normalized blood glucose in WD-fed mice without causing undesirable hypoglycemia in chow-fed mice (FIG. 5B). MLN4924 treatment decreased hepatic cullin neddylation and increased IRS protein and downstream AKT activation (FIG. 5N). Consistently, MLN4924 treatment decreased hepatic gluconeogenic gene glucose 6-phosphatase (G6Pase) but not phosphoenolpyruvate carboxylase (PEPCK). In addition, MLN4924-mediated downregulation of liver pyruvate kinase (L-PK), a carbohydrate response element binding protein (ChREBP) target gene, further serving as a marker for decreased circulating glucose in these mice. MLN4924 did not affect plasma insulin in chow-fed mice. Plasma insulin levels in WD-fed mice varied significantly and were not reduced by MLN4924. The lack of attenuated hyperinsulinemia in MLN4924-treated WD-fed mice led us to further investigate the potential effect of MLN4924 on pancreatic β cell insulin secretion. To this end, we used INS-1 832/13 rat pancreas β cells that have been commonly used to study glucose-stimulated insulin secretion. MLN4924 treatment did not affect basal or glucose-stimulated insulin secretion in INS-1 832/13 cells despite strong neddylation inhibition (FIG. 32), ruling out a direct β cell effect of MLN4924 that contributes to lower glucose.

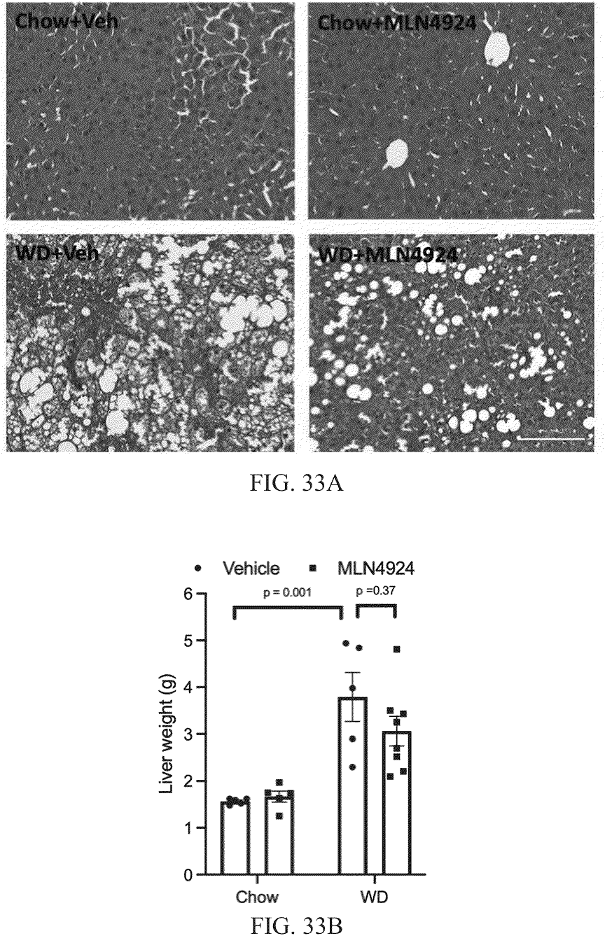

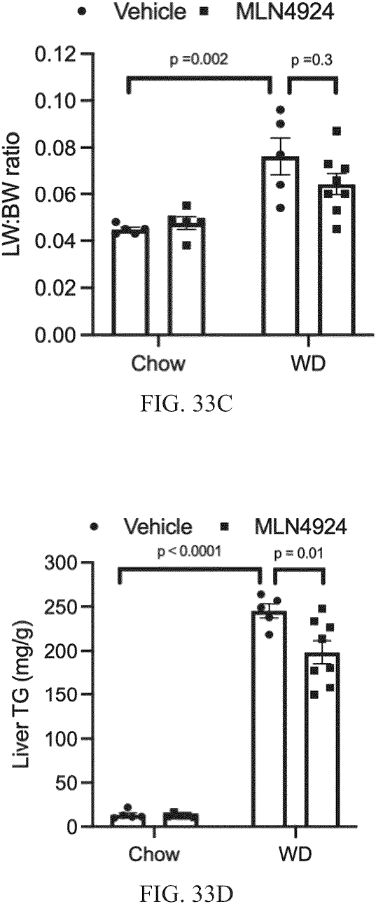

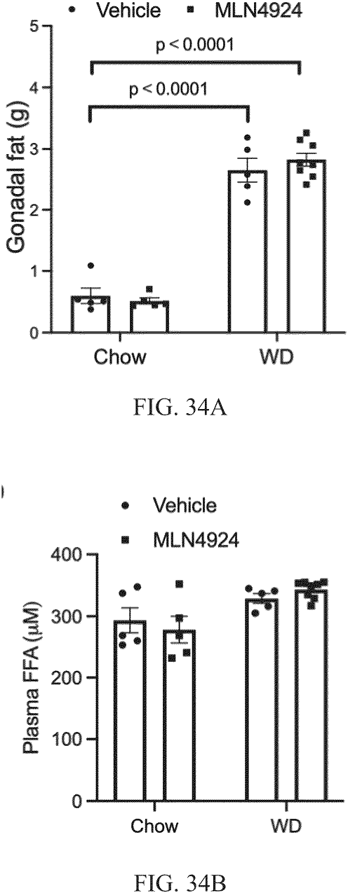

Further characterization revealed that MLN4924 treatment for 9 weeks did not cause alanine aminotransferase (ALT) elevation in chow-fed mice. Liver histology of chow-fed MLN4924-treated mice also appeared normal (FIG. 33A). These results suggest that chronic CRL inhibition by MLN4924 was well tolerated and did not cause hepatotoxicity. Interestingly, MLN4924 significantly reduced ALT in WD-fed mice, suggesting attenuated liver injury. This additional benefit of MLN4924 treatment may be partly explained by a significant reduction of hepatic fat accumulation (FIGS. 33A-33D). Reduced circulating glucose and hepatic L-PK expression indicated that reduced glucose-driven de novo lipogenesis may partially contribute to hepatic fat reduction independent of adiposity or circulating fatty acid changes (FIGS. 34A-34B). These results suggest that chronic MLN4924 treatment enhances insulin-mediated repression of hepatic glucose production without promoting insulin-driven lipogenesis, therefore antagonizing “selective hepatic insulin resistance,” a major pathogenic feature of fatty liver. Furthermore, targeting hepatic neddylation may also reduce fatty liver-associated liver cancer risk because cullin hyper-neddylation has been linked to liver cancer progression and poor prognosis. Given the versatile functions of CRLs, the absence of apparent adverse health impact may be due to partial inhibition of hepatic neddylation upon MLN4924 treatment and the lack of significant neddylation inhibition in other tissues such as the skeletal muscle.

In summary, disclosed herein is a novel insulin sensitizing function of an anti-cancer drug with a mechanism of action distinct from existing anti-diabetic drugs, therefore revealing a novel therapeutic strategy for insulin sensitization, increased insulin secretion, and hyperglycemia treatment. Mechanistically, treatment with MLN4924 and/or other NAE inhibitors (e.g., as described elsewhere herein) delays IRS protein degradation and insulin desensitization, which is largely attributed to attenuated Cul1 and Cul3 neddylation activation. The substrate specificity of a CRL is largely determined by the substrate receptor in the CRL complex. In at least certain embodiments, the present disclosure is directed to methods of increasing insulin secretion in a subject having a hyperglycemic condition. In some embodiments, the hyperglycemic condition is type-2 diabetes. The method may comprise administering to the subject an inhibitor compound which causes activity of Cullin RING E3 ligases (CRL) to be reduced, thereby causing an increase in insulin secretion followed by a subsequent decrease in blood glucose concentration in the subject. The inhibitor compound may be a neddylation inhibitor, such, for example, a NEDD8-activating enzyme (NAE) inhibitor, which may be an inhibitor of a UBA3 subunit of NAE. The neddylation inhibitor may inhibits a NEDD8 protein from being covalently linked to at least one of a cullin1 and a cullin3 protein of the CRL. The NAE inhibitor may be MLN4924 or active derivatives thereof, TAS4464, 6,6″biapigenin, cyclometalated rhodium (III) complexes, [Rh(ppy)₂(dppz)]⁺, [Rh(phq)₂(MOPIP)]⁺, Flavokawain A, Compound 1, Compound 13, ABP1, ABP A3, I-216, LZ3, Deoxyvasicinone derivatives, Piperacillin, Mitoxantrone, M22, LP0040, and ZM223, and salts of these compounds.

The NAE inhibitor may also be selected from those listed in the following US patent documents, including U.S. Pat. Publications: 2013/0116208 (e.g., ((1S,2S,4R)-4-(4-((1S)-2,3-dihydro-1H-inden-1-ylamino)-7H-pyrrolo[2,3-d]pyrimidin-7-yl)-2-hydroxycyclopentyl) methyl sulfamate (“MLN4924”) or {(1S,2S,4R)-4-[(6-{[(1R,2S)-5-chloro-2-methoxy-2,3-dihydro-1H-inden-1-yl]- amino}pyrimidin-4-yl)oxy]-2-hydroxycyclopentyl}methyl sulfamate (“I-216”), 2016/0160219 (e.g., siRNAs and shRNAs of Tables 1-3), 2016/0250303, 2017/0066772 (e.g., 4-amino-5-[2-(2,6-difluoro phenyl)ethynyl]-7-[(2R,3R,4S,5R)-3,4-dihydroxy-5-[(sulfamoylamino) methyl]-tetrahydrofuran-2-yl]pyrrolo[2,3-d]pyrimidine; 4-amino-5-[2-(4-amino-2,6-difluorophenyl)ethynyl]-7-[(2R,3R,4S,5R)-3,4-dihydroxy-5-[(sulfamoylamino)methyl]tetrahydrofuran-2-yl] pyrrolo[2,3-d]pyrimidine; 4-amino-5-[2-[2,6-difluoro-4-(methylamino)phenyl]ethynyl]-7-[(2R,3-R,4S,5R)-3,4-dihydroxy-5-[(sulfamoylamino)methyl]tetrahydrofuran-2-yl]pyrrolo[2,3-d] pyrimidine; 4-amino-5-[2-[2,6-difluoro-4-[(3R)-3-fluoropyrrolidin-1-yl]phenyl]ethynyl]-7-[(2R,3R, 4S,5R)-3,4-dihydroxy-5-[(sulfamoylamino)methyl]tetrahydrofuran-2-yl]pyrrolo[2,3-d]pyrimidine; 4-amino-7-[(2R,3R,4S,5R)-3,4-dihydroxy-5-[(sulfamoylamino)methyl]tetrahyd- rofuran-2-yl]-5-[2-(2-ethoxy-4,6-difluoro-phenyl)ethynyl]pyrrolo[2,3-d]pyr- imidine; 4-amino-5-[2-[2,6-difluoro-4-(3-hydroxyazetidin-1-yl)phenyl]ethynyl]-7-[(2R,3R,4S,5R)-3,4-dihydroxy-5-[(sulfamoylamino) methyl]tetrahydrofuran-2-yl]pyrrolo[2,3-d]pyrimidine; 4 amino-5-[2-[4-(azetidin-1-yl)-2,6-difluorophenyl]ethynyl]-7-[(2R,3R,4S,5-R)-3,4-dihydroxy-5[(sulfamoylamino) methyl]tetrahydrofuran-2-yl] pyrrolo[2,-3 -d]pyrimidine; 4-amino-7-[(2R,3R,4S,5R)-3,4-dihydroxy-5-[(sulfamoylamino) methyl]tetrahydrofuran-2-yl]-5-[2-(2-ethoxy-6-fluoro-phenyl)ethynyl]pyrrolo[2,3-d]pyrimidine; 4-amino-7-[(2R,3R,4S,5R)-3,4-dihydroxy-5-[(sulfamoylamino)methyl]tetrahydrofuran-2-yl]-5-[2-(2-fluoro-6-propoxy-phenyl)ethynyl]pyrrolo[2,3-d]pyrimidine; 8-[2-[4-amino-7-[(2R,3R,4S,5R)-3,4-dihydroxy-5-[(sulfamoylamino)-methyl]tetrahydrofuran-2-yl]pyrrolo[2,3-d]pyrimidin-5-yl] ethynyl]-7-fluoro-4-methyl-2,3-dihydro-1,4-benzoxazine; 4-amino-7-[(2R,3R,4S,5R)-3,4-dihydroxy-5-[(sulfamoylamino)methyl]tetrahydrofuran-2-yl]-5-[2-(2-ethylsulfanyl-6-fluoro-phenyl) ethynyl]pyrrolo[2,3-d]-pyrimidine; 4-amino-5-[2-[2-(cyclopropyl methoxy)-6-fluoro-phenyl] ethynyl]-7-[(2R,3R,4S,5R)-3,4-dihydroxy-5-[(sulf-amoylamino)methyl]tetrahydrofuran-2-yl] pyrrolo[2,3-d]pyrimidine; 4-amino-7-[(1R,2S,3R,4R)-2,3-dihydroxy-4-[(sulfamoylamino) methyl] cyclopentyl]-5-[2-(2-fluoro-6-methylsulfanyl-phenyl)ethynyl]pyrrolo[2,3-d]pyrimidine; 8-[2-[4-amino-7-[(1R,2S,3R,4R)-2,3-dihydroxy-4-[(sulfamoylamino)methyl]-cyclopentyl] pyrrolo [2,3-d]pyrimidin-5-yl]ethynyl]-7-fluoro-4-methyl-2,3-dihydro-1,4-benzoxazine; 4-amino-7-[(1R,4R,5S)-4,5-dihydroxy-3-[(sulfamoylamino)methyl]cyclopent-2-en-1-yl]-5-[2-(2-ethoxy-6-fluoro-phenyl) ethynyl]pyrrolo[2,3-d]pyrimidine; and 4-amino-7-[(1R,4R,5S)-4,5-dihydroxy-3-[(sulfamoyl amino)methyl]cyclopent-2-en-1-yl]-5-[2-(2-fluoro-6-methylsulfanyl-phenyl) ethynyl]pyrrolo[2,3-d-]pyrimidine; and salts of these compounds), 2017/0107579, 2018/0162864 (e.g., 4-amino-5-[2-(2,6-difluorophenyl)ethynyl]-7-[(2R,3R,4S,5R)-3,4-dihydroxy-5-[(sulfamoylamino)methyl]-tetrahydrofuran-2-yl]pyrrolo[2,3-d]pyrimidine; 4-amino-5-[2-(4-amino-2,6-difluoro-phenyl) ethynyl]-7-[(2R,3R,4S,5R)-3,4-dihydroxy-5-[(sulfamoylamino)methyl] tetrahydrofuran-2-yl]pyrrolo[2,3-d]pyrimidine; 4-amino-5-[2-[2,6-difluoro-4-(methylamino)phenyl]ethynyl]-7-[(2R,3R,4S,5R)-3,4-dihydroxy-5-[(sulfamoylamino)methyl]tetrahydrofuran-2-yl]pyrrolo[2,3-d]pyrimidine; 4-amino-5-[2-[2,6-difluoro-4-[(3R)-3-fluoro pyrrolidin-1-yl]phenyl]ethynyl]-7-[(2R,3R,4S,5R)-3,4-dihydroxy-5-[(sulfamoylamino)methyl]tetrahydrofuran-2-yl]pyrrolo[2,3-d]pyrimidine; 4-[4-[2-[4-amino-7-[(2R,3R,4S,5R)-3,4-dihydroxy-5-[(sulfamoylamino) methyl]tetrahydrofuran-2-yl]pyrrolo[2,3-d]pyrimidin-5-yl]ethynyl]-3-ethoxy-5-fluorophenyl]morpholine; 4-amino-7-[(2R,3R,4S,5R)-3,4-dihydroxy-5-[(sulfamoylamino)methyl] tetrahydrofuran-2-yl]-5-[2-(2-ethoxy-4,6-difluoro-phenyl)ethynyl]pyrrolo[2,3-d]pyrimidine; 4-[4-[2-[4-amino-7-[(2R,3R,4S,5R)-3,4-dihydroxy-5-[(sulfamoylamino)methyl]tetrahydrofuran-2-yl] pyrrolo[2,3-d]pyrimidin-5-yl]ethynyl]-3,5-difluoro-phenyl]thio morpholine; 4-amino-5-[2-[2,6-difluoro-4-(3-hydroxy azetidin-1-yl)phenyl]ethynyl]-7-[(2R,3R,4S,5R)-3,4-dihydroxy-5-[(sulfamoylamino)methyl] tetrahydrofuran-2-yl]pyrrolo[2,3-d]pyrimidine; 4-amino-5-[2-[4-(azetidin-1-yl)-2,6-difluoro-phenyl]ethynyl]-7-[(1R,2S,3R,4R)-2,3-dihydroxy-4-[(sulfamoylamino)methyl] cyclopentyl]pyrrolo[2,3-d]pyrimidine; 4-amino-7-[(2R,3R,4S,5R)-3,4-dihydroxy-5-[(sulfamoylamino)methyl] tetrahydrofuran-2-yl]-5-[2-(2-ethoxy-6-fluorophenyl)ethynyl] pyrrolo[2,3-d]pyrimidine; 4-amino-7-[(2R,3R,4S,5R)-3,4-dihydroxy-5-[(sulfamoylamino) methyl] tetrahydrofuran-2-yl]-5-[2-(2-fluoro-6-propoxyphenyl)ethynyl]pyrrolo[2,3-d] pyrimidine; 4-amino-7-[(2R,3R,4S,5R)-3,4-dihydroxy-5-[(sulfamoylamino)methyl] tetrahydrofuran-2-yl] -5-[2-[2-fluoro-6-(2,2,2-trifluoroethoxy)phenyl] ethynyl]pyrrolo[2,3-d] pyrimidine; 8-[2-[4-amino-7-[(2R,3R,4S,5R)-3,4-dihydroxy-5-[(sulfamoylamino)methyl] tetrahydrofuran-2-yl]pyrrolo[2,3-d]pyrimidin-5-yl] ethynyl]-7-fluoro-4-methyl-2,3-dihydro-1,4-benzoxazine; 4-amino-7-[(2R,3R,4S,5R)-3,4-dihydroxy-5-[(sulfamoylamino)methyl] tetrahydrofuran-2-yl]-5-[2-(2-ethylsulfanyl-6-fluorophenyl)ethynyl]pyrrolo[2,3-d]-pyrimidine; 4-amino-5-[2-[2-(cyclopropyl methoxy)-6-fluorophenyl]ethynyl]-7-[(2R,3R,4S,5R)-3,4-dihydroxy-5-[(sulfamoylamino)methyl] tetrahydrofuran-2-yl]pyrrolo[2,3-d]pyrimidine; 4-amino-7-[(1R,2S,3R,4R)-2,3-dihydroxy-4-[(sulfamoylamino)methyl] cyclopentyl]-5-[2-(2-ethoxy-6-fluorophenyl) ethynyl]pyrrolo[2,3-d]pyrimidine; 4-amino-7-[(1R,2S,3R,4R)-2,3-dihydroxy-4-[(sulfamoylamino)methyl]cyclopentyl]-5-[2-(2-fluoro-6-methylsulfanyl-phenyl)ethynyl] pyrrolo[2,3-d]pyrimidine; 8-[2-[4-amino-7-[(1R,2S,3R,4R)-2,3-dihydroxy-4-[(sulfamoylamino)methyl ]cyclopentyl]pyrrolo[2,3-d]pyrimidin-5-yl]ethynyl]-7-fluoro-4-methyl-2,3-dihydro-1,4-benzoxazine; 4-amino-7-[(1R,4R,5S)-4,5-dihydroxy-3 [(sulfamoylamino)methyl] cyclopent-2-en-1-yl]-5-[2-(2-ethoxy-6-fluorophenyl)ethynyl]pyrrolo[2,3-d]pyrimidine; 4-amino-7-[(1R,4R,5S)-4,5-dihydroxy-3-[(sulfamoylamino)methyl]cyclopent-2-en-1-yl]-5-[2-(2-fluoro-6-methylsulfanyl-phenyl)ethynyl]pyrrolo[2,- 3-d]pyrimidine; [(2R,3S,4R,5R)-5-[4-amino-5-[2-(2,6-difluoro phenyl)ethynyl]pyrrolo[2,3-d]pyrimidin-7-yl]-3,4-dihydroxytetrahydrofuran-2-yl] methyl sulfamate; 4-amino-5-[2-(2,6-difluorophenyl)ethynyl]-7-[(2R,3R,4S,5R)-3,4-dihydroxy-5-[(sulfamoylamino)methyl] tetrahydrofuran-2-yl]pyrrolo[2,3-d]pyrimidine; 4-amino-5-[2-(4-amino-2,6-difluoro-phenyl) ethynyl]-7-[(2R,3R,4S,5R)-3,4-dihydroxy-5-[(sulfamoylamino)methyl]tetrahydrofuran-2-yl]pyrrolo[2,3-d]pyrimidine; 4-amino-5-[2-[2,6-difluoro-4-(methylamino)phenyl]ethynyl]-7-[(2R,3R,4S,5R)-3,4-dihydroxy-5-[(sulfamoylamino)methyl] tetrahydrofuran-2-yl]pyrrolo[2,3-d]pyrimidine; 4-amino-5-[2-[2,6-difluoro-4-[(3R)-3-fluoro pyrrolidin-1-yl]phenyl]ethynyl]-7-[(2R,3R,4S,5R)-3,4-dihydroxy-5-[(sulfamoylamino)methyl]tetrahydrofuran-2-yl]pyrrolo[2,3-d]pyrimidine; 4-amino-7-[(2R,3R,4S,5R)-3,4-dihydroxy-5-[(sulfamoylamino)methyl]tetrahydrofuran-2-yl]-5-[2-(2-ethoxy-4,6-difluorophenyl)ethynyl]pyrrolo[2,3-d]pyrimidine; 4-amino-5-[2-[2,6-difluoro-4-(3-hydroxy azetidin-1-yl)phenyl]ethynyl]-7-[(2R,3R,4S,5R)-3,4-dihydroxy-5-[(sulfamoylamino)methyl]tetrahydrofuran-2-yl]pyrrolo[2,3-d]pyrimidine; 4-amino-5-[2-[4-(azetidin-1-yl)-2,6-difluorophenyl]ethynyl]-7-[(1R,2S,3R,4R)-2,3-dihydroxy-4-[(sulfamoylamino)methyl] cyclopentyl]pyrrolo[2,3-d]pyrimidine; 4-amino-7-[(2R,3R,4S,5R)-3,4-dihydroxy-5-[(sulfamoylamino)methyl]tetrahyd- rofuran-2-yl]-5-[2-(2-ethoxy-6-fluoro-phenyl)ethynyl] pyrrolo[2,3-d]pyrimidine; 4-amino-7-[(2R,3R,4S,5R)-3,4-dihydroxy-5[(sulfamoylamino)methyl] tetrahydrofuran-2-yl]-5-[2-(2-fluoro-6-propoxy-phenyl)ethynyl]pyrrolo[2,3-d]pyrimidine; 8-[2-[4-amino-7-[(2R,3R,4S,5R)-3,4-dihydroxy-5-[(sulfamoylamino)methyl]tetrahydrofuran-2-yl]pyrrolo[2,3-d]pyrimidin-5-yl]ethynyl]-7-fluoro-4-methyl-2,3-dihydro-1,4-benzoxazine; 4-amino-7-[(2R,3R,4S,5R)-3,4-dihydroxy-5-[(sulfamoylamino)methyl]tetrahydrofuran-2-yl]-5-[2-(2-ethylsulfanyl-6-fluorphenyl)ethynyl]pyrrolo[2,3-d]pyrimidine; 4-amino-5-[2-[2-(cyclopropyl methoxy)-6-fluorophenyl]ethynyl]-7-[(2R,3R,4S,5R)-3,4-dihydroxy-5-[(sulf-amoylamino) methyl]tetrahydrofuran-2-yl]pyrrolo[2,3-d]pyrimidine;4-amino-7-[(1R,2S,3R,4R)-2,3-dihydroxy-4-[(sulfamoylamino)methyl]cyclopentyl]-5-[2-(2-fluoro-6-methylsulfanyl-phenyl)ethynyl] pyrrolo[2,3-d]pyrimidine; 8-[2-[4-amino-7-[(1R,2S,3R,4R)-2,3-dihydroxy-4-[(sulfamoylamino)methyl]cyclopentyl]pyrrolo[2,3-d]pyrimidin-5-yl] ethynyl]-7-fluoro-4-methyl-2,3-dihydro-1,4-benzoxazine; 4-amino-7-[(1R,4R,5S)-4,5-dihydroxy-3[(sulfamoylamino)methyl] cyclopent-2-en-1-yl]-5-[2-(2-ethoxy-6-fluoro-phenyl)ethynyl] pyrrolo[2,3-d]pyrimidine; 4-amino-7-[(1R,4R,5S)-4,5-dihydroxy-3-[(sulfamoylamino)methyl] cyclopent-2-en-1-yl]-5-[2-(2-fluoro-6-methylsulfanyl-phenyl)ethynyl]pyrrolo[2,3-d] pyrimidine; and salts of these compounds), and 2018/0303833 (e.g., siRNAs, shRNAs, or miRNAs described in Table 1, which target the catalytic subunit UBA3 of NAE), each of which is expressly incorporated herein by reference in its entirety.

The present disclosure is directed, in at least certain embodiments, to the following:

Clause 1: A method of treating hyperglycemia in a subject having such a condition, comprising administering to the subject an inhibitor compound which causes activity of Cullin RING E3 ligases (CRL) to be reduced, wherein blood glucose concentration is decreased, and wherein the inhibitor compound is optionally administered as an inhibitor composition comprising a pharmaceutically-acceptable carrier, vehicle, or diluent.

Clause 2. The method of clause 1, wherein the subject has type-2 diabetes.

Clause 3. The method of clause 1 or 2, wherein the inhibitor compound is a neddylation inhibitor.

Clause 4. The method of clause 3, wherein the neddylation inhibitor is a NEDD8-activating enzyme (NAE) inhibitor.

Clause 5. The method of clause 4, wherein the NAE inhibitor is an inhibitor of a UBA3 subunit of NAE.

Clause 6. The method of any one of clauses 3-5, wherein the neddylation inhibitor inhibits a NEDD8 protein from being covalently linked to at least one of a cullin1 and a cullin3 protein of the CRL.

Clause 7. The method of any one of clauses 1-6, wherein insulin sensitization in the subject is increased.

Clause 8. The method of any one of clauses 1-7, wherein degradation of Insulin Receptor Substrate Protein 1 (IRS1) and Insulin Receptor Substrate Protein 2 (IRS2) is reduced and activation of Protein kinase B (AKT) is increased.

Clause 9. The method of clause 4 or 5, wherein the NAE inhibitor is a compound selected from the group consisting of MLN4924 and active derivatives thereof; TAS4464; 6,6″-biapigenin; cyclometalated rhodium (III) complexes; [Rh(ppy)₂(dppz)]⁺; [Rh(phq)₂(MOPIP)]⁺; Flavokawain A; Compound 1; Compound 13; ABP1; ABP A3; I-216; LZ3; Deoxyvasicinone derivatives; Piperacillin; Mitoxantrone; M22; LP0040; and ZM223; and salts of said NAE inhibitor compounds.

Clause 10. A composition comprising an inhibitor compound for use in treating hyperglycemia in a subject having such a condition, wherein the inhibitor compound causes activity of Cullin RING E3 ligases (CRL) to be reduced, thereby decreasing blood glucose concentration, and wherein the composition optionally comprises a pharmaceutically-acceptable carrier, vehicle, or diluent.

Clause 11. The composition of clause 10, wherein the composition is designed for use with a subject that has type-2 diabetes.

Clause 12. The composition of clause 10 or 11, wherein the inhibitor compound is a neddylation inhibitor.

Clause 13. The composition of clause 12, wherein the neddylation inhibitor is a NEDD8-activating enzyme (NAE) inhibitor.

Clause 14. The composition of clause 13, wherein the NAE inhibitor is an inhibitor of a UBA3 subunit of NAE.

Clause 15. The composition of clause 13 or 14, wherein the NAE inhibitor is a compound selected from the group consisting of MLN4924 and active derivatives thereof; TAS4464; 6,6″biapigenin; cyclometalated rhodium (III) complexes; [Rh(ppy)₂(dppz)]⁺; [Rh(phq)₂(MOPIP)]⁺; Flavokawain A; Compound 1; Compound 13; ABP1; ABP A3; I-216; LZ3; Deoxyvasicinone derivatives; Piperacillin; Mitoxantrone; M22; LP0040; and ZM223; and salts of said NAE inhibitor compounds.

Clause 16. The composition of any one of clauses 12-15, wherein the neddylation inhibitor inhibits a NEDD8 protein from being covalently linked to at least one of a cullin1 and a cullin3 protein of the CRL.

Clause 17. The composition of any one of clauses 10-16, wherein the inhibitor compound causes an increase in insulin sensitization in the subject.

Clause 18. The composition of any one of clauses 10-17, wherein the inhibitor compound causes a reduction in degradation of Insulin Receptor Substrate Protein 1 (IRS1) and Insulin Receptor Substrate Protein 2 (IRS2) and an increase in activation of Protein kinase B (AKT).

Clause 19: A method of increasing insulin secretion in a subject having a hyperglycemic condition, the method comprising administering to the subject an inhibitor compound which causes activity of Cullin RING E3 ligases (CRL) to be reduced, thereby causing an increase in insulin secretion followed by a subsequent decrease in blood glucose concentration in the subject, and wherein the inhibitor compound is optionally administered as an inhibitor composition comprising a pharmaceutically-acceptable carrier, vehicle, or diluent.

Clause 20. The method of clause 19, wherein the subject has type-2 diabetes.

Clause 21. The method of clause 19 or 20, wherein the inhibitor compound is a neddylation inhibitor.

Clause 22. The method of clause 21, wherein the neddylation inhibitor is a NEDD8-activating enzyme (NAE) inhibitor.

Clause 23. The method of clause 22, wherein the NAE inhibitor is an inhibitor of a UBA3 subunit of NAE.

Clause 24. The method of any one of clauses 21-23, wherein the neddylation inhibitor inhibits a NEDD8 protein from being covalently linked to at least one of a cullin1 and a cullin3 protein of the CRL.

Clause 25. The method of any one of clauses 19-24, wherein insulin sensitization in the subject is increased.

Clause 26. The method of any one of clauses 19-25, wherein degradation of Insulin Receptor Substrate Protein 1 (IRS1) and Insulin Receptor Substrate Protein 2 (IRS2) is reduced and activation of Protein kinase B (AKT) is increased.

Clause 27. The method of clause 22 or 23, wherein the NAE inhibitor is a compound selected from the group consisting of MLN4924 and active derivatives thereof; TAS4464; 6,6″biapigenin; cyclometalated rhodium (III) complexes; [Rh(ppy)₂(dppz)]⁺; [Rh(phq)₂(MOPIP)]⁺; Flavokawain A; Compound 1; Compound 13; ABP1; ABP A3; I-216; LZ3; Deoxyvasicinone derivatives; Piperacillin; Mitoxantrone; M22; LP0040; and ZM223; and salts of said NAE inhibitor compounds.

While several embodiments have been provided in the present disclosure, it will be understood that the disclosed methods and compositions can be embodied in many other specific forms without departing from the spirit or scope of the present disclosure. The present examples are to be considered as illustrative and not restrictive, and the intention is not to be limited to the details given herein. For example, the various compounds, compositions, elements or components may be combined or integrated in another method, composition, system or certain features may be omitted, or not implemented. In addition, inhibitor compounds, compositions, techniques, systems, subsystems, apparatus and methods described and illustrated in the various embodiments as discrete or separate may be combined or integrated with other compositions, systems, components, techniques, or methods without departing from the scope of the present disclosure. Other items or methods shown or discussed as coupled may be directly coupled or may be indirectly coupled or communicating through some interface, device, or intermediate component whether electrically, mechanically, or otherwise. Other examples of changes, substitutions, and alterations are ascertainable by one skilled in the art and may be made without departing from the spirit and scope disclosed herein.

What is claimed is:

1. A method of treating hyperglycemia in a subject having such a condition, comprising:
administering to the subject an inhibitor compound which causes activity of Cullin RING E3 ligases (CRL) to be reduced, wherein blood glucose concentration is decreased, and wherein the inhibitor compound optionally comprises a pharmaceutically-acceptable carrier, vehicle, or diluent.

2. The method of claim 1, wherein the subject has type-2 diabetes.

3. The method of claim 1, wherein the inhibitor compound is a neddylation inhibitor.

4. The method of claim 3, wherein the neddylation inhibitor is a NEDD8-activating enzyme (NAE) inhibitor.

5. The method of claim 4, wherein the NAE inhibitor is an inhibitor of a UBA3 subunit of NAE.

6. The method of claim 3, wherein the neddylation inhibitor inhibits a NEDD8 protein from being covalently linked to at least one of a cullin1 and a cullin3 protein of the CRL.

7. The method of claim 1, wherein administration of the inhibitor compound causes an increase in insulin sensitization in the subject.

8. The method of claim 1, wherein administration of the inhibitor compound causes a reduction in degradation of Insulin Receptor Substrate Protein 1 (IRS1) and Insulin Receptor Substrate Protein 2 (IRS2) and an increase in activation of Protein kinase B (AKT).

9. The method of claim 4, wherein the NAE inhibitor is a compound selected from the group consisting of TAS4464; 6,6″-biapigenin; cyclometalated rhodium (III) complexes; [Rh(ppy)2(dppz)]⁺; [Rh(phq)2(MOPIP)]⁺; Flavokawain A; Compound 1; Compound 13; ABP1; ABP A3; I-216; LZ3; Deoxyvasicinone derivatives; Piperacillin; Mitoxantrone; M22; LP0040; and ZM223; and salts of said NAE inhibitor compounds.

10. The method of claim 4, wherein the NAE inhibitor is a compound selected from the group consisting of MLN4924 and active derivatives thereof.

11. A method of increasing insulin secretion in a subject having a hyperglycemic condition, the method comprising:
administering to the subject an inhibitor compound which causes activity of Cullin RING E3 ligases (CRL) to be reduced, thereby causing an increase in insulin secretion followed by a subsequent decrease in blood glucose concentration in the subject.

12. The method of claim 11, wherein the subject has type-2 diabetes.

13. The method of claim 11, wherein the inhibitor compound is a neddylation inhibitor.

14. The method of claim 13, wherein the neddylation inhibitor is a NEDD8-activating enzyme (NAE) inhibitor.

15. The method of claim 14, wherein the NAE inhibitor is an inhibitor of a UBA3 subunit of NAE.

16. The method of claim 13, wherein the neddylation inhibitor inhibits a NEDD8 protein from being covalently linked to at least one of a cullin1 and a cullin3 protein of the CRL.

17. The method of claim 11, wherein administration of the inhibitor compound causes a reduction in degradation of Insulin Receptor Substrate Protein 1 (IRS1) and Insulin Receptor Substrate Protein 2 (IRS2) and an increase in activation of Protein kinase B (AKT).

18. The method of claim 4, wherein the NAE inhibitor is a compound selected from the group consisting of MLN4924 and active derivatives thereof; TAS4464; 6,6″-biapigenin; cyclometalated rhodium (III) complexes; [Rh(ppy)2(dppz)]⁺; [Rh(phq)2(MOPIP)]⁺; Flavokawain A; Compound 1; Compound 13; ABP1; ABP A3; I-216; LZ3; Deoxyvasicinone derivatives; Piperacillin; Mitoxantrone; M22; LP0040; and ZM223; and salts of said NAE inhibitor compounds.

19. A method of increasing insulin secretion in a subject having a hyperglycemic condition, the method comprising:
administering to the subject a compound selected from MLN4924, TAS4464, and pharmaceutically acceptable salts thereof.

20. The method of claim 19, wherein the subject has type-2 diabetes.
